SEQUENCE LISTING (4 sequences)

SEQ ID NO: 1 - DNA, 17 bp, Artificial Sequence
  Description of Artificial Sequence Synthetic primer
aatgaccccgaccgatt

SEQ ID NO: 2 - DNA, 19 bp, Artificial Sequence
  Description of Artificial Sequence Synthetic primer
gcacaagaggcaacgaagg

SEQ ID NO: 3 - DNA, 20 bp, Artificial Sequence
  Description of Artificial Sequence Synthetic primer
cgccgctagaggtgaaattc

SEQ ID NO: 4 - DNA, 19 bp, Artificial Sequence
  Description of Artificial Sequence Synthetic primer
ttggcaaatgctttcgctc

(19) United States
(12) Patent Application Publication
(10) Pub. No.: US 2006/0166947 A1
(43) Pub. Date: Jul. 27, 2006

(54) Multiple myeloma treatments

(76) Inventors: Kenneth C. Anderson, Wellesley, MA (US); Teru Hideshima, Brookline, MA (US)

Correspondence Address: BIOTECHNOLOGY LAW GROUP;C/O PORTFOLIOIP, PO BOX 52050, MINNEAPOLIS, MN, 55402, US

(21) Appl. No.: 11/267,031

(22) Filed: Nov. 4, 2005

Related U.S. Application Data
(63) Continuation-in-part of application No. 10/956,668, filed on Oct. 1, 2004.
(63) Continuation-in-part of application No. 10/957,039, filed on Oct. 1, 2004.
(60) Provisional application No. 60/667,088, filed on Mar. 30, 2005.
(60) Provisional application No. 60/625,323, filed on Nov. 5, 2004.

(51) Int. Cl. A61K 31/4745; A61K 31/407; A61K 31/403; A61K 31/192; A61K 31/60

(52) U.S. Cl. 514/165; 514/291; 514/411; 514/569; 514/570

(57) ABSTRACT

Provided herein are methods for treating refractory or resistant multiple myeloma in a subject using indole derivatives.

This application claims the benefit of, and priority to, each of the following U.S. provisional patent applications: Ser. Nos. 60/667,088, filed Mar. 30, 2005, and 60/625,323, filed Nov. 5, 2004, each entitled “Multiple Myeloma Treatments”. Each of these applications is incorporated herein by reference in its entirety, including figures and claims. The application is a also a continuation-in-part (CIP) of, and thus claims the benefit of, and priority to, U.S. patent application Ser. No. 10/956,668, entitled “Substituted Indole Derivatives”, and Ser. No. 10/957,039, entitled “Indole derivatives”, each filed Oct. 1, 2004, and each of which is also incorporated herein by reference in its entirety, including figures and claims.

### FIELD OF THE INVENTION

This invention relates to compounds and their use in treating multiple myeloma, in particular resistant or refractory multiple myeloma.

### BACKGROUND OF THE INVENTION

Multiple myeloma (MM) is a neoplastic disease in which malignant plasma cells accumulate in the bone marrow and secrete immunoglobulins (Ig's). The Ig's thus produced are homogeneous proteins called M proteins. The M proteins are observed in the blood in most cases. Known examples of these M proteins include IgG, IgA, BJP, IgD, IgE and IgM. Bence Jones Protein (BJP) is a protein comprising the L chain of Ig alone. The main focus of this disease resides in the bone marrow where the disease often results in bone lesions. In addition, common complications of multiple myeloma include recurrent bacterial infections, anemia, and renal insufficiency.

Multiple myeloma accounts for 7% of hematologic malignancies, with approximately 14,000 newly diagnosed cases in the United States annually. The median age of diagnosis is approximately 65 years, although 2% to 4% of patients are less than 40. Males are diagnosed approximately twice as frequently as females, and multiple myeloma is more common in blacks than in the white population. MM is responsible for approximately 1 percent of all cancer-related deaths in Western countries. Its etiology is unknown.

Chemotherapy is usually the preferred treatment for MM. Melphalan, cyclophosphamide, and glucocorticoids are commonly used as treatments. Melphalan and prednisone are considered the mainstay of treatment for MM and produce an objective response in 50-60% of patients. Other drugs have been shown to be effective for the treatment of MM including other alkylating agents (cyclophosphamide, BCNU), topoisomerase II inhibitors (doxorubicin and etoposide), glucocorticoids (prednisone and dexamethasone) and anti-tublin agents (vincristine). With the exception of the glucocorticoids, these chemotherapeutic agents are ineffective as single agents and are generally given in combination with other chemotherapeutic agents. Popular combinations include vincristine, carmustine, melphalan, cyclophosphamide and prednisone (VBMCP), vincristine, melphalan, cyclophosphamide, prednisone (VMCP) alternated with vincristine, carmustine, doxorubicin and prednisone. Although the majority of patients initially respond to treatment with chemotherapy and radiation, many will eventually suffer a relapse of the disease due to the proliferation of resistant tumor cells. Patients who become refractory have modest response rates to additional chemotherapy and a limited survival. The highest response rates in patients with MM resistant to alkylating agents are associated with the use of vincristine, doxorubicin and dexamethasone (VAD). Dexamethasone (Dex) can be used alone since it accounts for about 80% of the effect of VAD. The use of VAD alkylating resistant MM produces responses in 60-70% of patients but ultimately patients will develop resistance to all known chemotherapy regimens.

Thalidomide also has been used successfully in the treatment of multiple myeloma. Although the mechanism of action is unclear (may be related to a reduction in bone microvessel density or as a cytokine antagonist), thalidomide has been reported to yield a 40% to 50% response rate in patients with refractory disease. The most common toxicities of the agent are sedation and constipation. Thalidomide is contraindicated in pregnancy because it is highly teratogenic. Recent clinical trials have demonstrated that the combination of thalidomide and dexamethasone yields a 77% response rate.

The problem of drug resistance in MM is a major obstacle in curing MM. Thus, a need exists for new drugs and/or chemotherapeutic regimens for treating resistant MM.

Nonsteroidal anti-inflammatory drugs (NSAIDs) are commonly used for the treatment of inflammation, pain, and acute and chronic inflammatory disorders such as osteoarthritis and rheumatoid arthritis. These compounds are thought to work by inhibiting the enzyme cyclooxygenase (COX), which is also known as prostaglandin G/H synthase. COX catalyzes the conversion of arachidonic acid to prostaglandins.

Various forms of COX enzymes have been reported. They include a constitutive form known as COX-1, an inducible form known as COX-2 and the recently discovered COX-3, a variant of COX-1 that is inhibited by acetaminophen. COX-2 is inducible by mitogens, endotoxin, hormones, tumor promoters and growth factors. COX-1 is responsible for endogenous release of prostaglandins important for maintenance of gastrointestinal integrity and renal blood flow. Many of the side effects associated with NSAIDs are believed to be due to the inhibition of COX-1. Because of this, compounds that are selective for COX-2 have been developed and marketed. However, COX-2 inhibitors have been reported to cause dyspepsia, gastropathy and cardiovascular problems.

NSAIDs have also been used for cancer prevention and cancer treatment. The mechanism by which NSAIDs work in cancer treatment and cancer prevention may be related to COX overexpression. For example, some studies appear to indicate a link between COX expression and carcinogenesis. For example, cell lines that overexpress COX-2 are reported to be resistant to apoptosis, have increased invasiveness, and increased angiogenesis potential. Further, studies indicate that increased amounts of prostaglandins and COX-2 are commonly found in premalignant tissues and malignant tumors. Researchers have reported that COX-2 is up-regulated in several types of human cancers, including colon, pancreatic and breast.

Other studies report that the chemoprotective and antineoplastic properties of NSAIDs may occur in a COX-independent mechanism. For example, R-flurbiprofen is chemoprotective in the mouse model of intestinal polyposis and prostate cancer even though it does not have COX inhibitory activity. Similarly, sulindac sulfone, a metabolite of the NSAID sulindac, inhibits azoxy-methane-induced colon tumors in rats even though it does not have COX inhibitory activity. Further, NSAIDs can induce apoptosis in cancer cells that do not express COX-2 (Baek et al. 2001 Mol. Pharmacol. 59:901-908). The authors of these studies report that the chemoprotective and antineoplastic effects of NSAIDs occur via COX-dependent and COX-independent mechanisms.

β-catenin (also known as cadherin-associated protein) is a protooncogene in the downstream pathway of the wingless/frizzled (wnt/fzd) signaling pathway. Alterations in the pathways involved in regulating β-catenin are associated in the pathogenesis of many human cancers, including colorectal, desmoid (aggressive fibromatosis), endometrial, hepatocellular, leukemias, kidney, medulloblastoma, melanoma, ovarian, pancreatic, prostate, thyroid and uterine (Polakis, 2000 Genes Dev. 14:1837-1851; Chung et al. 2002 Blood 100:982-990).

β-catenin is reported to exist in at least three forms: membrane-bound (adherens complex), cytosolic, and nuclear. The nuclear accumulation of β-catenin, in concert with TCF/LEF proteins, induces downstream genes, including many genes implicated in tumorigenesis, for example, cyclin D1, and c-myc. The literature also reports that β-catenin is involved in the gene regulation of the androgen receptor, providing evidence for a role for the Wnt/β-catenin-TCF pathway for normal and neoplastic prostate growth (Amir et al., 2003, J. Biol. Chem. 278:30828-30834). The literature also reports that β-catenin may up-regulate COX-2 (Okamura et al., 2003, Cancer Res. 63:728-34).

β-catenin levels are reported to be regulated posttranslationally by the Wnt/fzd signaling pathway. In the absence of a Wnt signal, any β-catenin not bound to adherins is marked for degradation by a complex of proteins bound to β-catenin that includes glycogen synthase kinase-3β (GSK-3β), adenomatous polyposis coli (APC) protein, and axin. This complex facilitates the phosphorylation of β-catenin by GSK-3β and subsequent rapid degradation of β-catenin through proteasome degradation. Binding of Wnts to their receptors results in disruption of the β-catenin complex and inhibition of β-catenin degradation. This results in the accumulation of β-catenin in the cytoplasm and nucleus where it interacts with TCF/LEF proteins to regulate gene expression. Mutations in APC, β-catenin, or axin have been reported to increase the nuclear accumulation of β-catenin in cancers of epithelial origin.

The accumulation of β-catenin in the cytoplasm and nucleus has been reported in tumors with or without β-catenin mutations. In colorectal cancers, APC is mutated in 80% of all cases. In cases without APC mutations, β-catenin mutations are found in 50% of the cases. Accumulation of β-catenin is reported to occur in a very high percentage of cases in hepatoblastomas even though β-catenin is mutated in only 34% of the samples (Blaker et al., 1999 Genes Chromosomes Cancer 25:399-402). In hepatocellular carcinomas, β-catenin accumulation results from β-catenin mutations or axin mutation, but rarely APC mutations. Forty-two percent of samples in anaplastic thyroid demonstrate nuclear accumulation of β-catenin. Further, this high accumulation has been reported to correlate with a decrease in survival rate (Garcia-Rostan et al. 1999 Cancer Res. 59:1811-5). Rubinfeld et al. reported abnormal β-catenin regulation in 30% of melanoma cell lines (1997 Science 275:1790-2). Uterine endometriuim is reported to be associated with β-catenin accumulation in both samples that contain β-catenin mutations and samples without β-catenin mutations (Fukuchi et al. 1998 Cancer Res. 58:3526-3528.) Iwao et al. report that 63% of bone and soft-tissue tumors lacking a specific β-catenin mutation still demonstrate β-catenin accumulation (1999 Jpn. J. Cancer Res. 90:205-209).

Lin et al. reported that immunohistochemical analysis of cyclin D1 and β-catenin in breast tumors indicated that of 53 samples positive for cyclin D1, 49 of those were also β-catenin positive with β-catenin observed in both the nucleus and cytoplasm (2000 Proc. Natl. Acad. Sci. USA 97:4262-4266). A relationship between β-catenin and cyclin D1 has been reported for colon cancer and hepatocellular carcinoma (Tetsue et al. 1999 Nature 398:422-426; Ueta et al. 2002 Oncology Reports 9:1197-1203). Cyclin D1 is reported to be involved in the pathogenesis of squamous cell carcinoma (Xu et al. 1994 Int J. Cancer 59:383-387). It has also been reported that the expression of cyclin D1 is important for MM cell growth and prognosis (Hideshima et al 2004 Blood 104:607-618; Soverini et al. 2003 Blood 102:1588-1594).

NSAIDs have been reported to affect β-catenin activity. For example, both aspirin and indomethacin have been reported to inhibit transcription of the β-catenin/TCF target cyclin D1 (Dihlmann et al. 2001 Oncogene 20:645-53). Sulindac was reported to decrease β-catenin in intestinal tumors from Min/+mice (McEntee et al. 1999 Carcinogenesis 20:635-640). Noda et al., report that etodolac increases the expression and cytoplasmic accumulation of cytoplasmic E-cadherin in Caco2 cells, but had no quantitative change in β-catenin expression (2002 J. Gastorenterol. 37(11):896-904).

Peroxisome proliferators-activated receptors (PPARs) are nuclear hormone receptors that have been reported to be involved in many cellular processes, including lipid metabolism and disease-related processes. PPARs form dimers with retinoid-X receptor and mediate their effects after ligand binding through gene transcription.

Three isoforms of PPAR are known to date-α, γ, and δ. PPARα is highly expressed in liver and has been reported to stimulate lipid metabolism. PPARγ is highly expressed in adipose tissue and is reported to be involved in activating adipogeneisis. PPARγ is reported to be involved in insulin resistance and a number of neoplastic processes including colorectal cancer. Shimada et al. hypothesize that activation of PPARγ signaling may compensate for deregulated c-myc expression in cells with mutated APC (2002 Gut 50:658-664). Ohta et al. report that a PPARγ ligand can cause a shift in β-catenin from the nucleus to the cytoplasm and induction of differentiation in pancreatic cancer cells (2002 Int J. Oncol. 21:37-42). PPARδ is expressed in many tissues and organs with the highest expression are brain, colon, and skin. Investigators have implicated PPARδ in cholesterol efflux, colon cancer, embryo implantation, preadipocyte proliferation and epidermal maturation. Investigators report that PPARδ is a downstream target of β-catenin/TCF-4 transcription complex (He et al., 1999 Cell 99:335-345). Also, PPARδ mRNA is reported to be overexpressed in many colorectal cancers.

NSAIDs have been reported to activate PPAR receptors (Lehmann et al. 1997 J Biol. Chem. 272:3406-3410). Researchers also report that NSAIDs may inhibit PPARδ, which might contribute to the chemoprotective effects of NSAIDs in preventing colorectal cancers (He et al. 1999).

Epidemiological studies indicate that NSAIDs may reduce or prevent the occurrence of Alzheimer's disease. A connection between the COX pathway and Alzheimer's disease has been reported and is mainly based on epidemiological studies. Studies indicate that Cox-2 is up-regulated in areas of the brain related to memory (Hinz et al. 2002 J. Pharm. Exp. Ther. 300:367-375). Weggen et al. report that some NSAIDs may reduce the pathogenic amyloid β peptide, Aβ42, by as much as 80% (2001 Nature 414:212-216). This reduction has been reported to occur in a COX-independent mechanism (Eriksen et al. 2002 J. Clinical Invest. 112:440-449). Eriksen also report that flurbiprofen and its enantiomers lower Aβ42 by targeting the γ-secretase complex that produces Aβ from amyloid β protein precursor. U.S. Pat. No. 6,255,347 discloses the use of R-ibuprofen for the treatment or prevention of Alzheimer's disease.

Analogs of etodolac are known in the art see, for example, U.S. Pat. Nos. 5,830,911; 5,824,699; 5,776,967; 5,420,289; 4,748,252; 4,686,213; 4,070,371; 3,939,178; and 3,843,681.

The use of etodolac and enantiomers of etodolac to treat cancer is described in U.S. Pat. Nos. 6,573,292; 6,545,034; and 5,955,504.

The use of NSAIDs to treat inflammation, cancer, and angiogenesis have been reported in the art see, for example, see U.S. Pat. Nos. 5,972,986; 6,025,353; 5,955,504; and 5,561,151.

### SUMMARY OF THE INVENTION

The present invention provides methods for treating resistant or refractory multiple myeloma in a subject in need thereof.

Preferred compounds for treating resistant or refractory multiple myeloma include NSAIDS, analogs of NSAIDS, including those devoid of COX activity, compounds that modulate or inhibit β-catenin, and compounds that modulate or inhibit cyclin D.

Still other preferred compounds for treating resistant multiple myeloma include the compounds disclosed in U.S. Pat. Nos. 6,545,034; 6,573,292 and International Publication No. WO 02/12188; all of which are incorporated herein by reference in their entireties except to the extent they are inconsistent with the disclosures herein.

Compounds useful for treating resistant or refractory MM include compounds of Formula I. 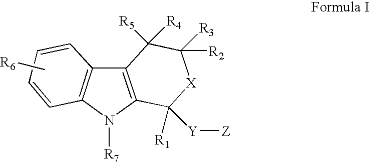
in which R₁ is selected from the group consisting of hydrogen, lower alkyl, lower alkenyl, lower alkynyl, lower cycloalkyl, phenyl, benzyl and 2-thienyl; R₂, R₃, R₄ and R₅ are the same or different and are each selected from the group consisting of hydrogen and lower alkyl, NH₂, —NHCHO, —NHCONH₂, ═NW, oxo, —OH and —OCH3, wherein W is hydroxy, alkoxy, aryloxy, carboxyalkyloxy, arylamino or alkylsulfonylamino; R₆ is selected from the group consisting of hydrogen, lower alkyl, lower alkenyl, lower alkynyl, trifluoromethyl, hydroxy, lower alkoxy, trifluoroloweralkoxy, benzyloxy, aralkoxy, aryloxy, lower alkanoyloxy, acyl, amino, nitro, cyano, alkylimido, halo, mercapto, loweralkylthio, alkylsulfinyl, alkylsulfonyl, alkylsulfonamido and sulfamoyl; R₇ is selected from the group consisting of hydrogen, lower alkyl and lower alkenyl; X is selected from the group consisting of carbon, oxy and thio; Y is selected from the group consisting of carbonyl, 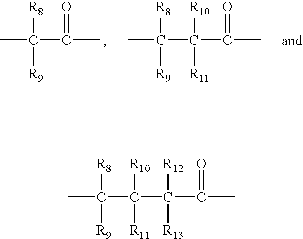
in which each of R₈, R₉, R₁₀, R₁₁, R₁₂ and R₁₃ is hydrogen or lower alkyl; and Z is selected from the group consisting of hydroxy, lower alkoxy, amino, lower alkylamino, di(lower)alkylamino and phenylamino,
or a pharmaceutically acceptable salt thereof.

Preferred compounds of Formula I are etodolac and the R isomer of etodolac (R-etodolac).

Additional compounds for treating resistant or refractory MM in a subject include indole compounds of Formula II: 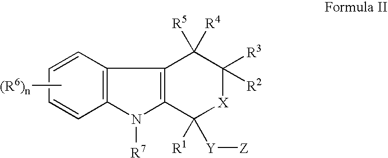
wherein R¹ is lower alkyl, lower alkenyl, (hydroxy)lower alkyl, lower alkynyl, phenyl, benzyl or 2-thienyl; R², R³, R⁴ and R⁵ are the same or different and are each hydrogen or lower alkyl; each R⁶ is independently hydrogen, lower alkyl, hydroxy, (hydroxy)lower alkyl, lower alkoxy, benzyloxy, lower alkanoyloxy, nitro or halo, wherein n is 1-3; R⁷ is hydrogen, lower alkyl or lower alkenyl; X is carbon, oxy or thio; Y is carbonyl, (CH₂)₁₋₃(O)—, —(CH₂)₁₋₃—, or —(CH₂)₁₋₃SO₂—; and Z is hydroxy, lower alkoxy, (C₂-C₄)acyloxy, —N(R⁸)(R⁹), phenylamino, (ω-(4-pyridyl)(C₂-C₄ alkoxy), (ω-((R⁸)(R⁹)amino)(C₂-C₄ alkoxy), an amino acid ester of (ω-(HO)(C₂-C₄))alkoxy, —N(R⁸)CH(R⁸)CO₂H, 1′-D-glucuronyloxy, —SO₃H, —PO₄H₂, —N(NO)(OH), —SO₂NH₂, —PO(OH)(NH₂), —OCH₂CH₂N(CH₃)₃⁺, or tetrazolyl; wherein R⁸ and R⁹ are each hydrogen, or (C₁-C₃)alkyl; or R⁸ and R⁹ together with N, form a 5- or 6-membered heterocyclic ring having 1-3 N(R⁸), S or non-peroxide O; or a pharmaceutically acceptable salt thereof.

Other preferred compounds for treating resistant or refractory MM include compounds of Formula III 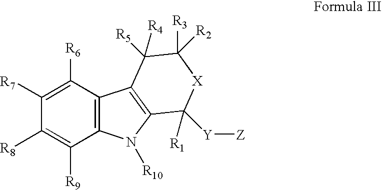
wherein:

(a) X is C, S or O;

(b) R₁ is hydrogen; halogen; —CN; —OH; —SH; —NO₂; or an unsubstituted or substituted moiety selected from alkyl, alkenyl, alkynyl, aryl, heteroaryl, heterocycloalkyl, and cycloalkyl, wherein the substituted groups are substituted with one, two or three suitable substituents each independently selected from the group consisting of: halogens, —CN, —NO₂, unsubstituted alkyl, unsubstituted alkenyl, unsubstituted heteroalkyl, unsubstituted haloalkyl, unsubstituted alkynyl, unsubstituted aryl, unsubstituted cycloalkyl, unsubstituted heterocycloalkyl, unsubstituted heteroaryl, and —(CH₂)_{z}CN where z is an integer from 0 to 6;

(c) R₂, R₃, R₄ and R₅ are each independently hydrogen; halogen; —CN; —OH; —SH; —NO₂; or an unsubstituted or substituted moiety selected from lower alkyl, lower alkynyl, lower alkenyl, alkoxy, haloalkyl, aryl, and heteroaryl;

(d) R₆, R₇, R₈ and R₉ are each independently hydrogen; halogen; —CN; —OH; —SH; —NO₂; or an unsubstituted or substituted moiety selected from alkyl, alkenyl, alkynyl, alkoxy, allyloxy, aryl, heteroaryl, cycloalkyl, and heterocycloalkyl, wherein at least one of R₆, R₇, R₈ and R₉ is an unsubstituted or substituted moiety selected from aryl, heteroaryl, cycloalkyl, heterocycloalkyl, alkenyl, and alkynyl;

(e) R₁₀ is hydrogen; or an unsubstituted or substituted moiety selected from lower alkyl, lower alkenyl, lower alkynyl, aryl; heteroaryl, heterocycloalkyl, and cycloalkyl;

(f) Y is an unsubstituted or substituted moiety selected from alkyl, alkenyl, and alkynyl; and

(g) Z is a moiety selected from —OH, —NH₂, —SH, —SO₂OH, —S(O)H, —OC(O)NH₂, —S(O)₂NH₂, —NHC(O)H, C(O)NH₂, unsubstituted or substituted with one, two or three suitable substituents each independently selected from the group consisting of alkyl, haloalkyl, heteroalkyl, aryl, heteroaryl, cycloalkyl, and heterocycloalkyl;

wherein R₁ and Y may cyclize to form an unsubstituted or substituted cycloalkyl group or an unsubstituted or substituted heterocycloalkyl group;

or a pharmaceutically acceptable prodrug, pharmaceutically active metabolite, or pharmaceutically acceptable salt thereof.

In another aspect of the invention, a method for treating refractory or resistant multiple myeloma includes administering to a subject in need thereof a compound of Formula (III) 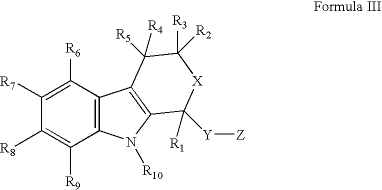
wherein:
(a) X is C, S or O;
(b) R₁ is hydrogen; halogen; —CN; —OH; —SH; —NO₂; or an unsubstituted or substituted moiety selected from alkyl, alkenyl, alkynyl, aryl, heteroaryl, heterocycloalkyl, and cycloalkyl, wherein the substituted groups are substituted with one, two or three suitable substituents each independently selected from the group consisting of: halogens, —CN, —NO₂, unsubstituted alkyl, unsubstituted alkenyl, unsubstituted heteroalkyl, unsubstituted haloalkyl, unsubstituted alkynyl, unsubstituted aryl, unsubstituted cycloalkyl, unsubstituted heterocycloalkyl, unsubstituted heteroaryl, and —(CH₂)_{z}CN where z is an integer from 0 to 6;
(c) R₂, R₃, R₄ and R₅ are each independently hydrogen; halogen; —CN; —OH; —SH; —NO₂; or an unsubstituted or substituted moiety selected from lower alkyl, lower alkynyl, lower alkenyl, alkoxy, haloalkyl, aryl, and heteroaryl;
(d) R₆, R₈ and R₉ are each independently hydrogen; halogen; —CN; —OH; —SH; —NO₂; or an unsubstituted or substituted moiety selected from alkyl, alkenyl, alkynyl, alkoxy, allyloxy, aryl, heteroaryl, cycloalkyl, and heterocycloalkyl;
(e) R₇ is hydrogen; halogen; —CN; —OH; —SH; —NO₂; or an unsubstituted or substituted moiety selected from alkyl, alkenyl, and alkynyl.
(f) R₁₀ is hydrogen; or an unsubstituted or substituted moiety selected from lower alkyl, lower alkenyl, lower alkynyl, aryl; heteroaryl, heterocycloalkyl, and cycloalkyl;
(g) Y is an unsubstituted or substituted moiety selected from alkyl, alkenyl, and alkynyl; wherein the substituted moiety is substituted with one, two or three substituents each independently selected from halogen; —CN; —OH; —SH; —NO₂; unsubstituted alkyls, unsubstituted alkenyls, unsubstituted alkynyls, unsubstituted heteroalkyls, unsubstituted haloalkyls, unsubstituted aryls, unsubstituted cycloalkyls, unsubstituted heterocycloalkyls, and unsubstituted heteroaryls; and
(h) Z is a moiety selected from —OH, —SH, —OC(O)NH₂;

wherein R₁ and Y may cyclize to form an unsubstituted or substituted cycloalkyl group or an unsubstituted or substituted heterocycloalkyl group; and at least one of R₆, R₇, R₈ and R₉ is not hydrogen; or a pharmaceutically acceptable salt thereof.

In yet another aspect of the invention, a method for treating refractory or resistant multiple myeloma includes administering to a subject in need thereof a compound of Formula (III) 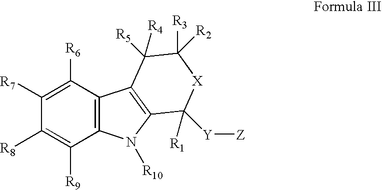
wherein:
(a) X is C, S or O;
(b) R₁ is hydrogen; halogen; —OH; —SH; —CN; —NO₂; or an unsubstituted or substituted moiety selected from alkyl, alkenyl, alkynyl, aryl, heteroaryl, heterocycloalkyl, and cycloalkyl, wherein the substituted groups are substituted with one, two or three suitable substituents each independently selected from the group consisting of: halogens, —CN, —NO₂, —SH, —OH, unsubstituted alkyl, unsubstituted alkenyl, unsubstituted heteroalkyl, unsubstituted haloalkyl, unsubstituted alkynyl, unsubstituted aryl, unsubstituted cycloalkyl, unsubstituted heterocycloalkyl, unsubstituted heteroaryl, and —(CH₂)_{z}CN where z is an integer from 0 to 6;
(c) R₂, R₃, R₄ and R₅ are each independently hydrogen; halogen; —OH; —SH; —CN; —NO₂; or an unsubstituted or substituted moiety selected from lower alkyl, lower alkynyl, lower alkenyl, alkoxy, haloalkyl, aryl, and heteroaryl;
(d) R₆, R₇, R₈ and R₉ are each independently hydrogen; halogen; —OH; —SH; —CN; —NO₂; or an unsubstituted or substituted moiety selected from alkyl, alkenyl, alkynyl, alkoxy, allyloxy, aryl, heteroaryl, cycloalkyl, and heterocycloalkyl, wherein at least one of R₆, R₇, R₈ and R₉ is an unsubstituted or substituted moiety selected from aryl, heteroaryl, cycloalkyl, heterocycloalkyl, alkenyl, and alkynyl;
(e) R₁₀ is hydrogen; or an unsubstituted or substituted moiety selected from lower alkyl, lower alkenyl, lower alkynyl, aryl; heteroaryl, heterocycloalkyl, and cycloalkyl;
(f) Y is an unsubstituted or substituted moiety selected from alkyl, alkenyl, and alkynyl; wherein the substituted moiety is substituted with one, two or three substituents each independently selected from halogen; —OH; —SH; —CN; —NO₂; unsubstituted alkyls, unsubstituted haloalkyls, unsubstituted heteroalkyls, unsubstituted alkenyls, unsubstituted alkynyls, unsubstituted aryls, unsubstituted cycloalkyls, unsubstituted heterocycloalkyls, and unsubstituted heteroaryls; and
(g) Z is a moiety selected from —OH, —SH, —OC(O)NH₂, —SO₂H, —SO₂NH₂, —SO₂OH, —S(O)H, —NH₂, —NHC(O)H, C(O)NH₂, unsubstituted or substituted with one or two suitable substituents selected from the group consisting of alkyl, haloalkyl, heteroalkyl, aryl, heteroaryl, cycloalkyl, and heterocycloalkyl each independently substituted with one, two or three suitable substituents;
   wherein R₁ and Y may cyclize to form an unsubstituted or substituted cycloalkyl group or an unsubstituted or substituted heterocycloalkyl group; or pharmaceutically acceptable salt thereof.

In some embodiments, the substituted groups in R₂, R₃, R₄, R₅, R₆, R₇, R₈, R₉ and R₁₀ of Formula III are substituted with one, two or three suitable substituents each independently selected from the group consisting of: halogens, ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 0 to 6, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one, two or three suitable substituents each independently selected from the group consisting of halogens, ═O, —NO2, —CN, —OH, —SH, —(CH2)z-CN where z is an integer from 0 to 6, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)O_{c}, —OC(O)NR_{c}R_{c}, —SRc, unsubstituted alkyls, unsubstituted alkenyls, unsubstituted alkynyls, unsubstituted heteroalkyls, unsubstituted haloalkyls, unsubstituted aryls, unsubstituted cycloalkyls, unsubstituted heterocycloalkyls, and unsubstituted heteroaryls, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

In other embodiments, the substituted groups in R₂, R₃, R₄, R₅, R₆, R₇, R₈, R₉, and R₁₀ of Formula III are substituted with one, two or three suitable substituents each independently selected from the group consisting of: halogens, ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 0 to 6, ═NH, —OH, —C(O)H, —OC(O)H, —C(O)OH, —OC(O)OH, —C(NH)NH₂, —NHC(O)NH₂, —S(O)H, —NH₂, —C(O)NH₂, —OC(O)NH₂, —NHC(O)H, —NHC(O)OH, —C(S)H, and —SH groups unsubstituted or substituted with one, two or three suitable substituents each independently selected from the group consisting of halogens, ═O, —NO2, —CN, —OH, —SH, —(CH2)z-CN where z is an integer from 0 to 6, unsubstituted alkyls, unsubstituted alkenyls, unsubstituted alkynyls, unsubstituted heteroalkyls, unsubstituted haloalkyls, unsubstituted aryls, unsubstituted cycloalkyls, unsubstituted heterocycloalkyls, and unsubstituted heteroaryls.

Another aspect of the invention includes treating subjects having resistant or refractory multiple myeloma with a compound of Formula III wherein

(a) X is C, S or O;

(b) R₁ is hydrogen; halogen; —CN; —NO₂; —OH; —SH; or an unsubstituted or substituted moiety selected from alkyl, alkenyl, alkynyl, aryl, heteroaryl, heterocycloalkyl, and cycloalkyl;

(c) R₂, R₃, R₄ and R₅ are each independently hydrogen; halogen; —CN; —NO₂; —OH; —SH; or an unsubstituted or substituted moiety selected from lower alkyl, lower alkynyl, lower alkenyl, alkoxy, haloalkyl, aryl, and heteroaryl;

(d) R₆, R₇, R₈ and R₉ are each independently hydrogen; halogen; —CN; —NO₂; —OH; —SH; or an unsubstituted or substituted moiety selected from alkyl, alkenyl, alkynyl, alkoxy, allyloxy, aryl, heteroaryl, cycloalkyl, and heterocycloalkyl, wherein at least one of R₆, R₇, R₈ and R₉ is an unsubstituted or substituted moiety selected from aryl, heteroaryl, cycloalkyl, heterocycloalkyl, alkenyl, and alkynyl;

(e) R₁₀ is hydrogen; or an unsubstituted or substituted moiety selected from lower alkyl, lower alkenyl, lower alkynyl, aryl; heteroaryl, heterocycloalkyl, and cycloalkyl;

(f) Y is an unsubstituted or substituted moiety selected from alkyl, alkenyl, and alkynyl; and

(g) Z is a moiety selected from —OH, —NH₂, —SH, —S(O)₂NH₂, —SO₂OH, —S(O)H, —NHC(O)H, C(O)NH₂, unsubstituted or substituted with one or two suitable substituents selected from the group consisting of alkyl, haloalkyl, heteroalkyl, aryl, heteroaryl, cycloalkyl, and heterocycloalkyl each independently substituted with one, two or three suitable substituents;

wherein R₁ and Y may cyclize to form an unsubstituted or substituted cycloalkyl group or an unsubstituted or substituted heterocycloalkyl group;

or a pharmaceutically acceptable prodrug, pharmaceutically active metabolite, or pharmaceutically acceptable salt thereof.

In yet another aspect of the invention, a method for treating refractory or resistant multiple myeloma includes administering to a subject in need thereof a compound of Formula III wherein:
(a) X is S or O;
(b) R₁ is hydrogen; or an unsubstituted moiety selected from lower alkyl, lower alkyl-hydroxy, lower alkenyl, lower alkenyl-hydroxy, lower alkynyl, lower alkynyl-hydroxy, aryl, heteroaryl, heterocycloalkyl, and cycloalkyl;
(c) R₂, R₃, R₄ and R₅ are each independently hydrogen; or an unsubstituted moiety selected from lower alkyl, lower alkynyl, lower alkenyl, alkoxy, haloalkyl, aryl, and heteroaryl;
(d) R₆, R₈ and R₉ are each independently hydrogen; or an unsubstituted or substituted moiety selected from alkyl, alkenyl, alkynyl, alkoxy, allyloxy, aryl, heteroaryl, cycloalkyl, and heterocycloalkyl, wherein the substituted moieties are each independently selected from the group consisting of halogen, —CN, alkyl, alkoxy, —NH₂, —O-haloalkyl, —CH(O), haloalkyl, aryl, heteroaryl, heterocycloalkyl, alkenyl, alkynyl, —OH, —C(O)₂-alkyl, and —C(O)₂H;
(e) R₇ is hydrogen; halogen; —CN; —OH; —SH; —NO₂; unsubstituted lower alkyl, unsubstituted lower alkenyl, unsubstituted lower alkynyl, alkyl-C(O)₂H, alkyl-C(O)₂-alkyl, or lower alkoxy; and
(f) R₁₀ is hydrogen; or an unsubstituted moiety selected from lower alkyl, lower alkenyl, lower alkynyl, aryl, benzyl, heteroaryl, heterocycloalkyl, and cycloalkyl.

In still yet another aspect of the invention, a method for treating refractory or resistant multiple myeloma includes administering to a subject in need thereof a compound of Formula III wherein:
(a) R₉ is hydrogen, halogen or an unsubstituted alkyl group;
(b) Y is an unsubstituted alkyl group; and
(c) Z is hydroxyl.

In yet another aspect of the invention, a method for treating refractory or resistant multiple myeloma includes administering to a subject in need thereof a compound of Formula III wherein:
(a) X is O or S;
(b) R₁ is an unsubstituted lower alkyl group;
(c) R₂, R₃, R₄ and R₅ are each hydrogen;
(d) R₆ is hydrogen or halogen;
(e) R₇ is halogen, unsubstituted lower alkyl, lower alkyl-C(O)₂H, lower alkyl-C(O)₂-lower alkyl, or lower alkoxy;
(f) R₈ is hydrogen or halogen;
(g) R₉ is hydrogen; or an unsubstituted or substituted moiety selected from alkenyl, alkynyl, aryl, heteroaryl, cycloalkyl, and heterocycloalkyl; and
(h) R₁₀ is hydrogen.

In a further aspect of the invention, a method for treating refractory or resistant multiple myeloma includes administering to a subject in need thereof a compound of Formula III wherein:
(a) X is O;
(b) R₁ is an unsubstituted moiety selected from aryl, alkyl, and lower-alkoxy;
(c) R₂, R₃, R₄, and R₅ are each hydrogen;
(d) R₆ and R₈ are each hydrogen or halogen;
(e) R₇ is halogen, unsubstituted lower alkyl, lower alkyl-C(O)₂H, lower alkyl-C(O)₂-lower alkyl, or lower alkoxy;
(f) R₉ is an unsubstituted branched alkyl group; and
(g) R₁₀ is hydrogen;

In some embodiments, the substituted groups in R₂, R₃, R₄, R₅, R₆, R₇, R₈, R₉ and R₁₀ of Formula III are substituted with one, two or three suitable substituents each independently selected from the group consisting of: halogens, ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 0 to 6, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one, two or three suitable substituents each independently selected from the group consisting of halogens, ═O, —NO₂, —CN, —(CH₂)_{z}—CN where z is an integer from 0 to 6, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyls, unsubstituted haloalkyls, unsubstituted heteroalkyls, unsubstituted alkenyls, unsubstituted alkynyls, unsubstituted aryls, unsubstituted cycloalkyls, unsubstituted heterocycloalkyls, and unsubstituted heteroaryls, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

In other embodiments, the substituted groups in R₂, R₃, R₄, R₅, R₆, R₇, R₈, R₉, and R₁₀ of Formula III are substituted with one, two or three suitable substituents each independently selected from the group consisting of: halogens, ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 0 to 6, ═NH, —OH, —C(O)H, —OC(O)H, —C(O)OH, —OC(O)OH, —C(NH)NH₂, —NHC(O)NH₂, —S(O)H, —NH₂, —C(O)NH₂, —OC(O)NH₂, —NHC(O)H, —NHC(O)OH, —C(S)H, and —SH groups unsubstituted or substituted with one, two or three suitable substituents each independently selected from the group consisting of halogens, ═O, —NO₂, —CN, —(CH₂)_{z}—CN where z is an integer from 0 to 6, unsubstituted alkyls, unsubstituted haloalkyls, unsubstituted heteroalkyls, unsubstituted alkenyls, unsubstituted alkynyls, unsubstituted aryls, unsubstituted cycloalkyls, unsubstituted heterocycloalkyls, and unsubstituted heteroaryls. or a pharmaceutically acceptable prodrug, pharmaceutically active metabolite, or pharmaceutically acceptable salt thereof.

Exemplary compounds within Formula III useful for the methods described herein are shown below:

| NO. | STRUCTURE |
|---|---|
| 1 | 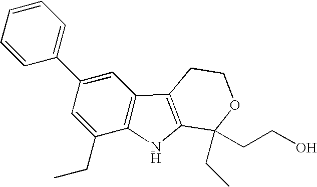 |
| 2 | 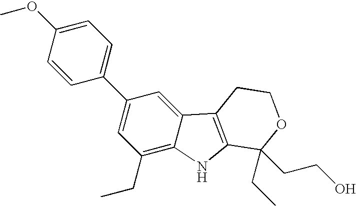 |
| 3 | 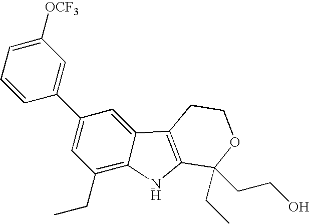 |
| 4 | 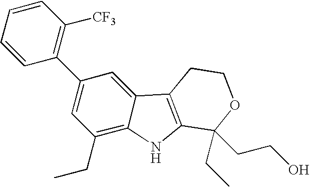 |
| 5 | 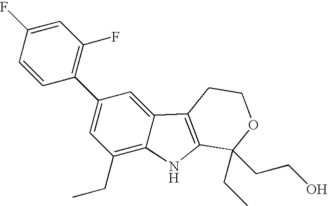 |
| 6 | 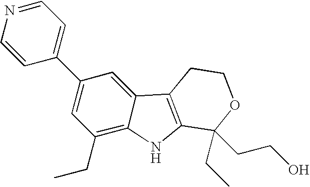 |
| 7 | 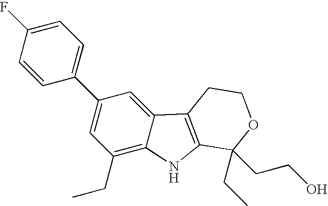 |
| 8 | 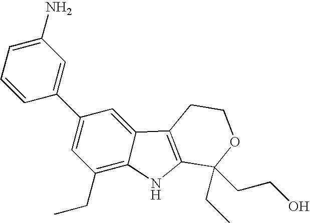 |
| 9 | 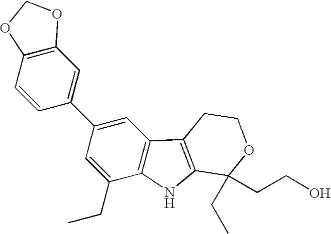 |
| 10 | 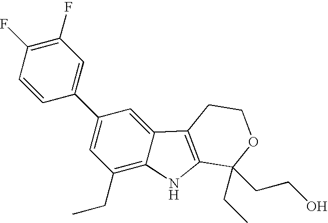 |
| 11 | 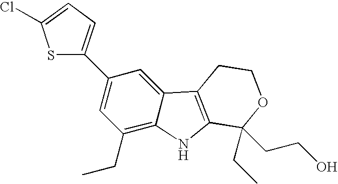 |
| 12 | 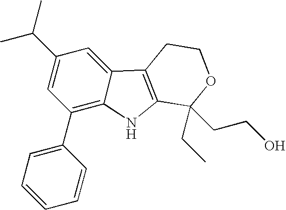 |
| 13 | 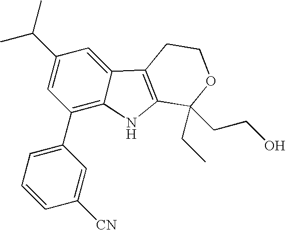 |
| 14 | 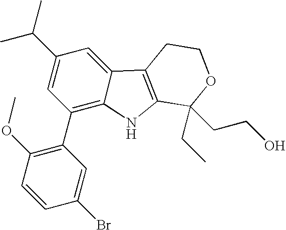 |
| 15 | 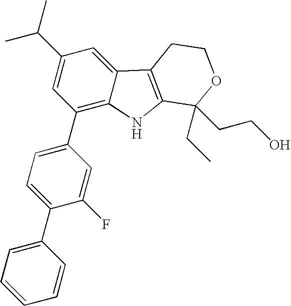 |
| 16 | 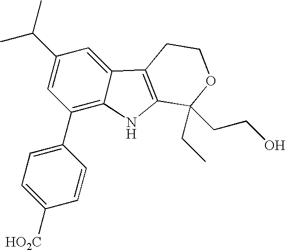 |
| 17 | 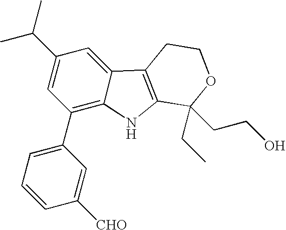 |
| 18 | 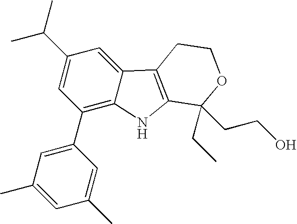 |
| 19 | 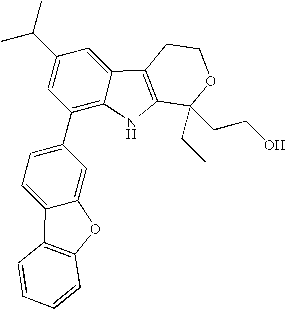 |
| 20 | 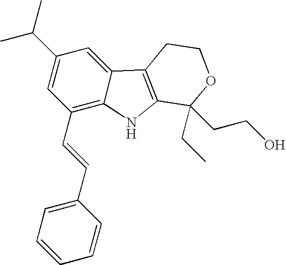 |
| 21 | 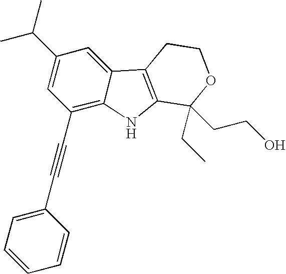 |
| 22 | 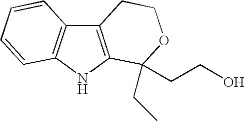 |
| 23 | 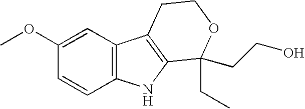 |
| 24 | 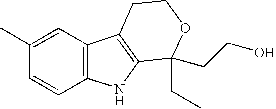 |
| 25 | 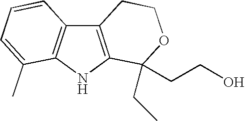 |
| 26 | 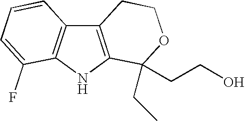 |
| 27 | 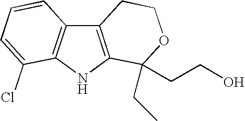 |
| 28 | 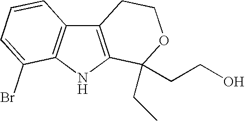 |
| 29 | 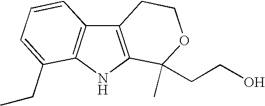 |
| 30 | 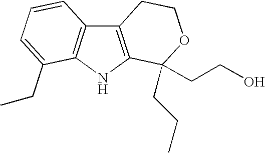 |
| 31 | 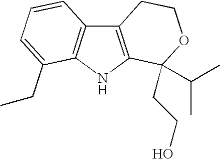 |
| 32 | 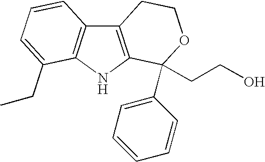 |
| 33 | 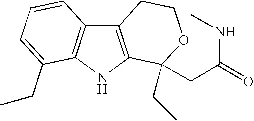 |
| 34 | 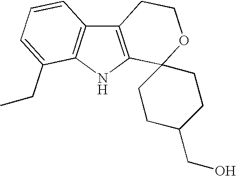 |
| 35 | 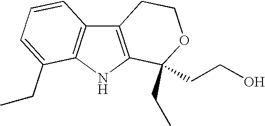 |
| 36 | 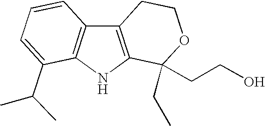 |
| 37 | 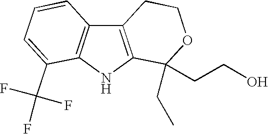 |
| 38 | 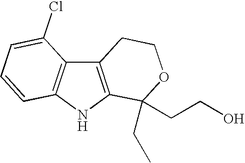 |
| 39 | 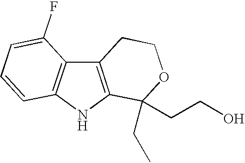 |
| 40 | 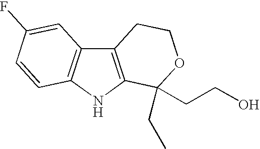 |
| 41 | 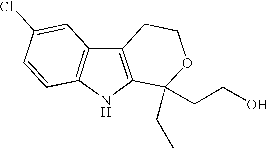 |
| 42 | 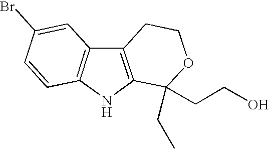 |
| 43 | 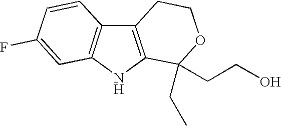 |
| 44 | 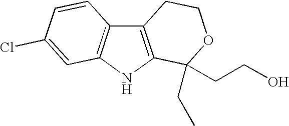 |
| 45 | 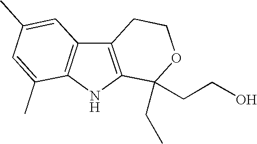 |
| 46 | 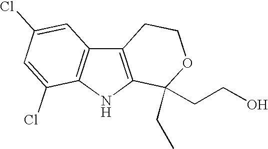 |
| 47 | 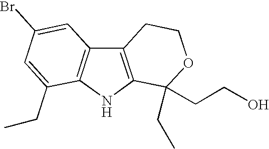 |
| 48 | 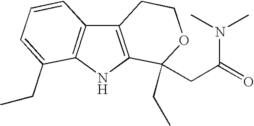 |
| 49 | 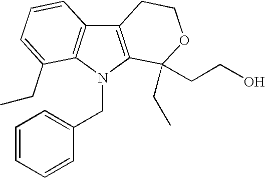 |
| 50 | 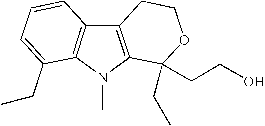 |
| 51 | 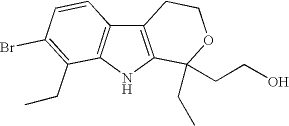 |
| 52 | 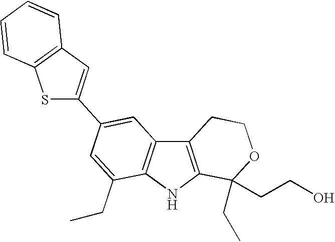 |
| 53 | 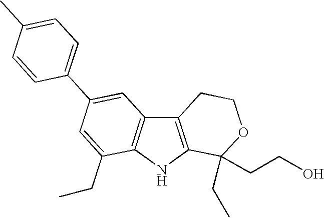 |
| 54 | 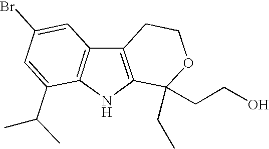 |
| 55 | 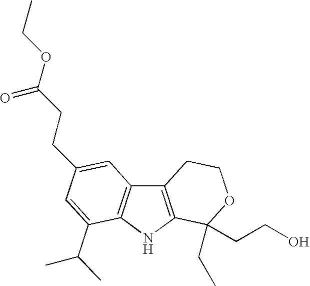 |
| 56 | 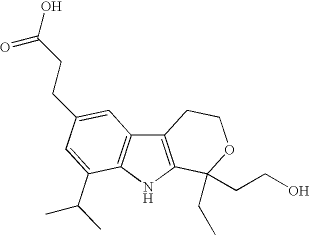 |
| 57 | 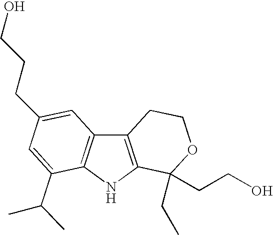 |

or pharmaceutically acceptable salts thereof.

Another embodiment of the invention includes treatment of refractory or resistant multiple myeloma by the administration of the compounds of Formulas I, II, III, or IV to a subject in need thereof in combination with one or more antineoplastic agents. Preferred antineoplastic agents include one or more of the following: vincristine, doxorubicin, dexamethasone, thalidomide, thalidomide derivatives, 2ME2, Neovastat, R 11 5777, arsenic trioxide, bortezomib, tamoxifen, G3139 (antisense), and SU5416.

Another aspect of the invention includes a method for treating refractory or resistant multiple myeloma by administering to a subject that has failed VAD therapy a compound of Formula I, II, III, or IV.

The invention also involves methods of treating resistant or refractory multiple myeloma by administering to a patient an antineoplastic agent that induces upregulation of Mcl-1s. Preferred compounds for such treatment are compounds selected from formulas I, II, III, or IV. Preferred compounds for such treatment are R-etodolac and compound 47.

Another embodiment of the invention includes methods for selecting a compound for use in treating multiple myeloma wherein the method involves contacting multiple myeloma cells with a test compound and determining if said compound induces the upregulation of Mcl-1s, wherein if said compound induces upregulaton of Mcl-1s it is a candidate as a drug for use in treating multiple myeloma including multiple myeloma resistant to antineoplastic agents including agents such as dexamethasone.

The preferred subject for the methods of the present invention is a human.

The methods of the invention include pharmaceutical compositions comprising a therapeutically effective amount of a prodrug, active metabolite, or pharmaceutically acceptable salt of a compound of Formulas I, II, III, and IV, as well as pharmaceutically acceptable salts of such active metabolites, are also provided herein. Thus, a related aspect of the invention concerns the use of NSAID or NSAID analogs or enantiomers (e.g., a prodrug, active metabolite, or compound of Formulas I, II, III, and IV, or a pharmaceutically acceptable salt thereof) in the manufacture of medicaments for treating refractory or resistant multiple myeloma.

### BRIEF DESCRIPTION OF THE DRAWINGS

FIG. 1 shows inhibition of β-catenin:TOP flash by R-etodolac and compounds of the invention.

FIG. 2(A) shows inhibition of Cyclin D1 mRNA expression by R-etodolac and compounds of the invention. 2(B) shows the effect of R-etodolac on cyclin D1 protein expression in U266 cells. U266 cells were cultured with or without R-etodolac (1.0 mM) for the indicated times. Total cell lysates were subjected to Western blotting using anti-cyclin D1 and α-tubulin antibodies. 2(C) shows the cell cycle profile of U266 cells treated with R-etodolac.

FIG. 3 shows the cytotoxic effects of R-etodolac (SDX-101) on drug-sensitive and drug-resistant MM cell lines. 3(A) shows the effect of SDX-101 on MM.1S and MM.1R cells (MM.1S (●), MM.1R (▪). 3(B) shows the effect of SDX-101 on MM.1S, U266, and RPMI8226 cells MM.1S (●), U266 (▪), RPMI8226 (▴). 3(C) shows the effect of SDX-101 on RPM18226, LR5, and Dox40 cells (RPM18226 (●), LR5 (▪), Dox40 (▴). 3D shows the effect of SDX-101 on DHL4 cells [DHL4 (♦), MM.1S (▴)].

FIG. 4 shows the effects of SDX-101 on normal peripheral blood mononuclear cells (PBMC).

FIG. 5 shows cytotoxic effects of SDX-101 in combination with conventional and novel agents in MM.1S.

FIG. 6 shows that SDX-101 overcomes the stimulating effect of IL-6 and IGF-1 on MM.1S and RPM18226 cells.

FIG. 7 shows a cell cycle analysis of MM.1S cells, using propidium iodide staining, after treatment with various concentrations of SDX-101.

FIG. 8-shows that apoptosis triggered by SDX-101 is mediated by via Caspase-8, Caspase-3 and PARP cleavage. In 8(A), MM.1S cells were cultured for 24 h with R-etodolac (0-1.25 mM); In 8(B), cells were cultured with R-etodolac (0.6 mM) for the indicated times. In 8(C), MM.1S cells were pre-incubated with Z-VAD-FMK (25 μM) for 30 min prior to treatment with R-etodolac (0.6 mM) for the indicated times. In 8(D), RPM18226 cells were also cultured with or without R-etodolac (0.6 mM) for the indicated times. Total cell lysates were subjected to Western blotting using anti-caspase-8, -9, -3, PARP, and α-tubulin Abs. CL stands for cleaved.

FIG. 9 shows that the effects of SDX-101 on BAX, Bcl-2, p53 and p21.

FIG. 10-shows the effect of SDX-101 on MM.1S cells co-cultured with BMSCs cells.

FIG. 11-shows that SDX-101 induces upregulation of Mcl-1ₛ and Dex-induced apoptosis in MM cells. 11(A)—MM.1S cells were cultured with SDX-101 at the indicated doses for 24 hours. 11(B)—MM.1S cells were cultured with SDX-101 (0.6 mM) for the indicated times. 11C— MM.1S cells were cultured for 24 hours with control media or 1.0 μM Dex, in the presence or absence of SDX-101 (0.15 or 0.3 mM). Total cell lystates were subjected to Western Blotting using anticaspase-8, -9, PARP, Bax, Bcl-xL, Mcl-1 and α-tubulin Abs.

FIG. 12 shows that SDX-101 induces apoptosis in patient MM cells. 12(A)—CD138 positive cells were isolated from BM of MM patients (MM #1 and MM #2) who had relapsed and were refractory to conventional therapies. MM #1 (♦), MM #2 (▪) and MM.1S cell line (▴) were cultured for 48 h in the presence of R-etodolac (0-2.5 mM). Cell growth was assessed by MTT assays, and data represent mean (±SD) of triplicate cultures. 12(B)—MM #1 and MM#2 cells were cultured with R-etodolac (0.6 mM) for 24 h. Total cell lysates were subjected to immunoblotting using anti-caspase-8 and PARP Abs.

FIG. 13 shows that SDX-101 induces growth inhibition in Dex resistant MM cells. 13(A)—OPM1 cells were cultured in control media (●) and with 0.1 μM (▴) or 1.0 μM (▪) of Dex for indicated times. Cell growth was assessed by MTT assays, and data represent mean±standard deviation (SD) of quadruplicate cultures. 13(B)—OPM1 cells were cultured with control media and with 0.1 or 1.0 μM Dex for 24 h, and then incubated in control media (white bars) and with 0.3 mM (gray bars) or 0.6 mM (black bars) of R-etodolac for other 24 h.

FIG. 14 shows that SDX-101 enhances Dex-induced upregulation of IκBα expression 14(A)—OPM1 cells were cultured with Dex (0.01-0.1 μM) for 24 h; and 14(B) with R-etodolac (0.15 or 0.3 mM) for 24 h. 14(C)—OPM1 cells were cultured for 24 h in control media and with 0.01 μM Dex, in the presence or absence of 0.15 or 0.3 mM R-etodolac. Total cell lysates were subjected to immunoblotting using anti-IκBα Abs. The density of the bands was assessed by Scion Image Beta, which converts the band area into pixels.

FIG. 15 shows that SDX-101 enhances Dex-induced activation of caspases. OPM1 cells were cultured for 12 h in control media and with 1.0 μM Dex, in the presence or absence of 0.3 or 0.6 mM R-etodolac. Total cell lysates were subjected to immunoblotting using anti-caspase-8, -9, and PARP Abs.

FIG. 16 shows the effect of SDX-101 combined to Dex on MM cell growth in vivo. 16(A)—CB-17 SCID mice were injected s.c. with 2×10⁶ OPM1 cells and when tumors were measurable daily treatment was started. Mice groups were control group (n=4), Dex 1 mg/kg (n=4), R-etodolac 250 mg/kg (n=4), and R-etodolac plus Dex (n=4). Tumor volume was estimated in two dimensions using an electronic caliper and the volume was expressed in mm³ using the following formula V=0.5a×b² where a and b are the long and short diameter of the tumor respectively. The P value was calculated by comparison between control and combination treatment group. Points indicate mean, bars standard error (SE). 16(B)—In vivo growth inhibitory effects of R-etodolac and/or Dex are expressed as the percentage of control value. SQ was calculated at the same time point (SQ>1 indicates a synergistic effect); bars indicate SE.

FIG. 17 shows that compound 47 induced cytotoxicity on MM cells as well as botezomib-resistant hematopoetic cancer cells. (A) MM.1S (♦), MM.1R (▪), OPM1 (▴), U266 (-), and INA6 (●) MM cells; (B) RPM18226 (♦), doxorubicin-resistant RPM18226-Dox40 (▪), and melphalan-resistant RPMI8226-LR5 MM cells (▴); (C) MM.1S (♦), bortezomib-resistant DHL4 (♦), and bortezomib-resistant KG1 (●) cells were cultured for 48 h in the presence of SDX-308 (0-100 μM). Cell growth was assessed by MTT assays, and data represent mean SD) of quadruplicate cultures.

FIG. 18 (A) shows the effects of compound 47 on normal peripheral blood mononuclear cells (PBMC). (B) shows that compound 47 has more than 10-fold active potential than R-etodolac. (A) Peripheral blood mononuclear cells from healthy volunteer; #1 (♦) #2 (▪) and #3 (▴). All cells were cultured for 48 h in the presence of compound 47 (0-100 μM). Cell growth was assessed by MTT assays, and data represent mean (±SD) of triplicate cultures.

### DETAILED DESCRIPTION OF THE INVENTION

To more readily facilitate an understanding of the invention and its preferred embodiments, the meanings of terms used herein will become apparent from the context of this specification in view of common usage of various terms and the explicit definitions of other terms provided in the glossary below or in the ensuing description.

Glossary of Terms

As used herein, the terms “comprising,” “including,” and “such as” are used in their open, non-limiting sense.

As used herein and in the appended claims, the singular forms “a”, “an”, and “the” include plural reference unless the context clearly dictates otherwise.

In accordance with a convention used in the art, 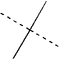
is used in structural formulae herein to depict the bond that is the point of attachment of the moiety or substituent to the core or backbone structure.

In accordance with a convention used in the art, the symbol 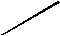
represents a methyl group, 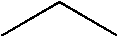
represents an ethyl group, 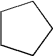
represents a cyclopentyl group, etc.

The term “alkyl” as used herein refers to a straight- or branched-chain alkyl group having one to twelve carbon atoms. Exemplary alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and the like. The term “lower alkyl” designates an alkyl having from 1 to 6 carbon atoms (a C₁₋₆-alkyl).

The term “heteroalkyl” as used herein refers to straight- and branched-chain alkyl groups having from one to twelve atoms containing one or more heteroatoms selected from S, O, and N. The term “lower heteroalkyl” designates a heteroalkyl having from 1 to 6 carbon atoms (a C₁₋₆-heteroalkyl).

The term “alkenyl” means an alkyl radical having one or more double bonds and two to twelve carbon atoms. Alkenyl groups containing three or more carbon atoms may be straight or branched. Alkenyl groups as used herein include either the cis or trans configurations. Illustrative alkenyl groups include prop-2-enyl, but-2-enyl, but-3-enyl, 2-methylprop-2-enyl, hex-2-enyl, and the like. The term “lower alkenyl” designates an alkyl having from 1 to 6 carbon atoms (a C₁₋₆-alkenyl).

The term “allyloxy” refers to an alkenyloxy group which is CH₂═CHCH₂—O—.

The term “alkynyl” means an alkyl radical having one or more triple bonds and two to twelve carbon atoms. Alkynyl groups containing three or more carbon atoms may be straight or branched. Alkynyl groups as used herein include either the cis or trans configurations. Illustrative alkynyl groups include prop-2-ynyl, but-2-ynyl, but-3-ynyl, 2-methylbut-2-ynyl, hex-2-ynyl, and the like. The term “lower alkynyl” designates an alkyl having from 1 to 6 carbon atoms (a C₁₋₆-alkynyl).

The term “aryl” (Ar) refers to a monocyclic, or fused or spiro polycyclic, aromatic carbocycle (ring structure having ring atoms that are all carbon) having from three to twelve ring atoms per ring. Illustrative examples of aryl groups include the following moieties: 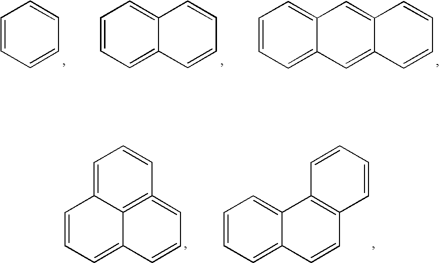
and the like.

The term “heteroaryl” (heteroAr) refers to a monocyclic, or fused or spiro polycyclic, aromatic heterocycle (ring structure having ring atoms selected from carbon atoms as well as nitrogen, oxygen, and sulfur heteroatoms) having from three to twelve ring atoms per ring. Illustrative examples of heteroaryl groups include the following moieties: 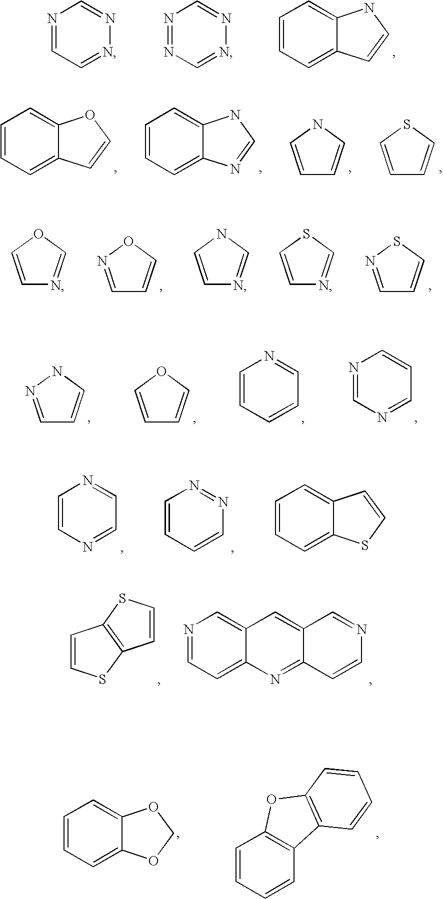
and the like.

The term “cycloalkyl” refers to a saturated or partially saturated, monocyclic or fused or spiro polycyclic, carbocycle having from three to twelve ring atoms per ring. Illustrative examples of cycloalkyl groups include the following moieties: 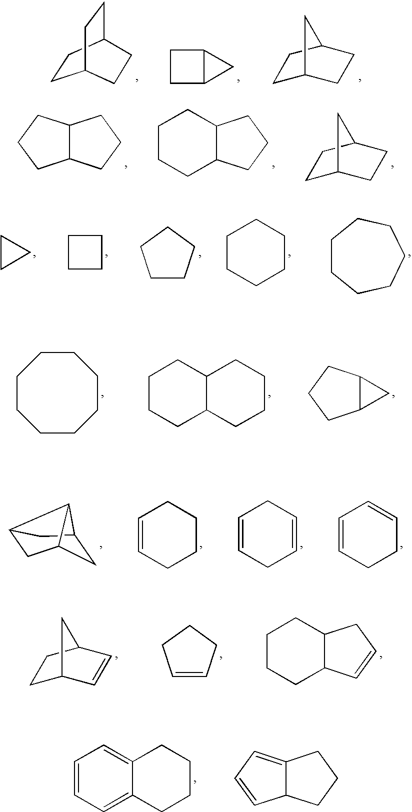
and the like.

A “heterocycloalkyl” refers to a monocyclic, or fused or spiro polycyclic, ring structure that is saturated or partially saturated and has from three to twelve ring atoms per ring selected from C atoms and N, O, and S heteroatoms. Illustrative examples of heterocycloalkyl groups include: 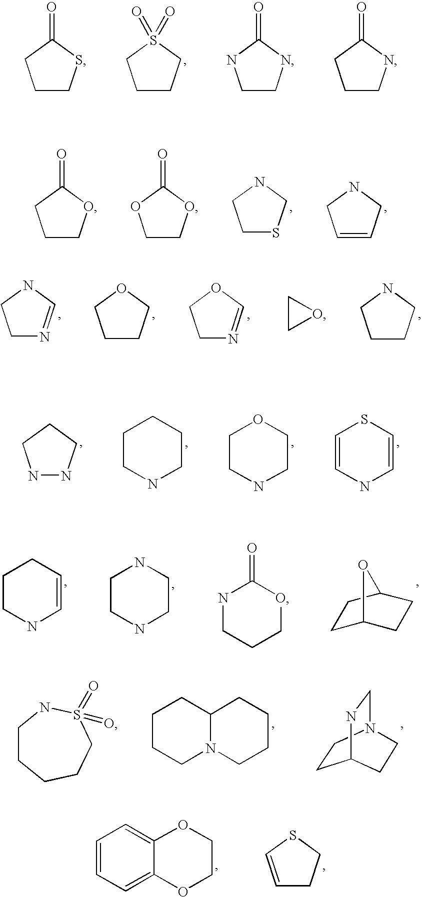
and the like.

The term “alkoxy” refers to O-alkyl. Illustrative examples include methoxy, ethoxy, propoxy, and the like.

The term “halogen” represents chlorine, fluorine, bromine or iodine. The term “halo” represents chloro, fluoro, bromo or iodo.

Unless otherwise defined, the term “substituted” as used herein means at least one hydrogen atom is replaced with a suitable substituent.

The term “unsubstituted” means that the specified group bears no substituents.

The term “lower” when referring to a group such as an alkyl, alkenyl, alkynyl, alkoxy or other group refers to such a group having up to 6 carbon atoms.

The term “subject” for purposes of treatment includes any human or animal subject who has any one of the known diseases or conditions described herein, e.g., multiple myeloma, preferably resistant or refractory multiple myeloma. For methods of prevention, the subject is any human or animal subject, and preferably is a human subject who is at risk for the disease or conditions described herein, e.g., cancer. Besides being useful for human treatment, the compounds described herein are also useful for veterinary treatment of mammals, including companion animals and farm animals, such as horses, dogs, cats, cows, sheep and pigs. Preferably, subject means a human.

The term “NSAID analog” as used herein is intended to mean a compound derived from the parent structure of an NSAID compound, i.e., an analog derived from a nonsteroidal anti-inflammatory drug that has some activity against COX-1 and/or COX-2 or reduces pain. The analog may exhibit COX activity or analgesic activity but may also be devoid of any COX activity or analgesic activity. An NSAID analog may be derived from another NSAID analog.

“A pharmaceutically acceptable salt” is intended to mean a salt that retains the biological effectiveness of the free acids and bases of the specified compound and that is not biologically or otherwise undesirable.

The terms “treating”, “treat” and “treatment” refer to any treatment of multiple myeloma in a mammal, particularly a human, and include: (i) preventing the disease or condition from occurring in a subject which may be predisposed to the condition such that the treatment constitutes prophylactic treatment for the pathologic condition; (ii) modulating or inhibiting the disease or condition, i.e., arresting its development; (iii) relieving the disease or condition, i.e., causing regression of the disease or condition; or (iv) relieving and/or alleviating disease or condition or the symptoms resulting from the disease or condition, without addressing the underlining disease or condition.

A therapeutically effective dose further refers to that amount of one or more compounds of the instant invention sufficient to result in treatment of the disorder.

The phrase “conjunctive therapy” (or “combination therapy”) refers to a therapeutic regimen that involves the provision of at least two distinct therapies to achieve an indicated therapeutic effect. In the context of use of a compound of the invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent. Alternatively, a combination therapy may involve the administration of one or more compound of the invention as well as the delivery of radiation therapy, stem cell transplantation, immunotherapy, and/or surgery or other techniques (e.g., nutritional therapy, naturopathic therapy, etc.) to either improve the quality of life of the patient or to treat the cancer. On the other hand, “monotherapy” refers to a treatment regimen based on the delivery of one therapeutically effective compound, whether administered as a single dose or several doses over time.

In the context of the commercialization of pharmaceuticals, the terms “promotion”, “promote”, “promoting”, and the like refer to any and all informational, persuasive, and scientific activities conducted by or on behalf of a manufacturer, distributor, or other entity involved in the discovery, research, development, and/or commercialization of the particular pharmaceutical compound, composition, or treatment regimen intended, directly or indirectly, to induce the prescription, supply, purchase, and/or use of the compound, composition, or treatment regimen. Such activities may be directed toward anyone in the in the supply and distribution chain, including, without limitation, medical professionals (e.g., physicians and nurses), pharmacists, health care administrators, insurance company or government representatives, and patients (including potential patients). In other words, the primary aim of promotion is to stimulate the sale or use of, and/or interest in, a particular pharmaceutical compound, composition, or treatment regimen, and thus any activity intended to serve this aim constitutes “promotion” of the particular pharmaceutical compound, composition, or treatment regimen.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

In accordance with the present invention, methods are provided for the treatment of resistant or refractory multiple myeloma (MM) in a subject comprising administering to said subject a therapeutically effective amount of an indole derivative of the Formulae I, II, III, and IV. 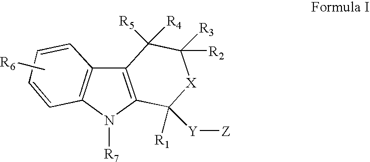
in which R₁ is selected from the group consisting of hydrogen, lower alkyl, lower alkenyl, lower alkynyl, lower cycloalkyl, phenyl, benzyl and 2-thienyl; R₂, R₃ R₄ and R₅ are the same or different and are each selected from the group consisting of hydrogen and lower alkyl, NH₂, —NHCHO, —NHCONH₂, ═NW, oxo, —OH and —OCH3, wherein W is hydroxy, alkoxy, aryloxy, carboxyalkyloxy, arylamino or alkylsulfonylamino; R₆ is selected from the group consisting of hydrogen, lower alkyl, lower alkenyl, lower alkynyl, trifluoromethyl, hydroxy, lower alkoxy, trifluoroloweralkoxy, aryloxy, benzyloxy, aralkoxy, lower alkanoyloxy, acyl, amino, nitro, cyano, alkylimido, halo, mercapto, loweralkylthio, alkylsulfinyl, alkylsulfonyl, alkylsulfonamido and sulfamoyl; R₇ is selected from the group consisting of hydrogen, lower alkyl and lower alkenyl; X is selected from the group consisting of carbon, oxy and thio; Y is selected from the group consisting of carbonyl, 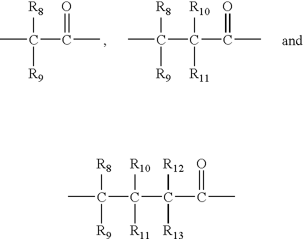
in which each of R₈, R₉, R₁₀, R₁₁, R₁₂ and R₁₃ is hydrogen or lower alkyl; and Z is selected from the group consisting of hydroxy, lower alkoxy, amino, lower alkylamino, di(lower)alkylamino and phenylamino,
or a pharmaceutically acceptable salt thereof.

Additional compounds for treating resistant or refractory MM in a subject include indole compounds of Formula II: 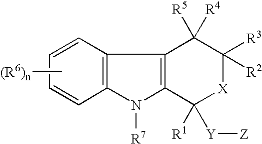
wherein R¹ is lower alkyl, lower alkenyl, (hydroxy)lower alkyl, lower alkynyl, phenyl, benzyl or 2-thienyl; R², R³, R⁴ and R⁵ are the same or different and are each hydrogen or lower alkyl; each R⁶ is independently hydrogen, lower alkyl, hydroxy, (hydroxy)lower alkyl, lower alkoxy, benzyloxy, lower alkanoyloxy, nitro or halo and n is 1-3; R⁷ is hydrogen, lower alkyl or lower alkenyl; X is carbon, oxy or thio; Y is carbonyl, (CH₂)₁₋₃C(O)—, —(CH₂)₁₋₃—, or —CH₂)₁₋₃SO₂—; and Z is hydroxy, lower alkoxy, (C₂-C₄)acyloxy, —N(R⁸)(R⁹), phenylamino, (ω-(4-pyridyl)(C₂-C₄ alkoxy), (ω-((R⁸)(R⁹)amino)(C₂-C₄ alkoxy), an amino acid ester of (ω-(HO)(C₂-C₄))alkoxy, —N(R⁸)CH(R⁸)CO₂H, 1′-D-glucuronyloxy, —SO₃H, —PO₄H₂, —N(NO)(OH), —SO₂NH₂, —PO(OH)(NH₂), —OCH₂CH₂N(CH₃)₃⁺, or tetrazolyl; wherein R⁸ and R⁹ are each hydrogen, or (C₁-C₃)alkyl; or R⁸ and R⁹ together with N, form a 5- or 6-membered heterocyclic ring having 1-3 N(R⁸), S or non-peroxide O; or a pharmaceutically acceptable salt thereof.

Additional compounds for treating resistant or refractory MM include compounds of Formula III 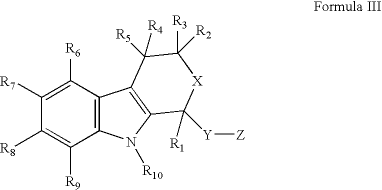
wherein:

(a) X is C, S or O;

(b) R₁ is hydrogen; halogen; —CN; —OH; —SH; —NO₂; or an unsubstituted or substituted moiety selected from alkyl, alkenyl, alkynyl, aryl, heteroaryl, heterocycloalkyl, and cycloalkyl, wherein the substituted groups are substituted with one, two or three suitable substituents each independently selected from the group consisting of: halogens, —CN, —NO₂, unsubstituted alkyl, unsubstituted alkenyl, unsubstituted heteroalkyl, unsubstituted haloalkyl, unsubstituted alkynyl, unsubstituted aryl, unsubstituted cycloalkyl, unsubstituted heterocycloalkyl, unsubstituted heteroaryl, and —(CH₂)_{z}CN where z is an integer from 0 to 6;

(c) R₂, R₃, R₄ and R₅ are each independently hydrogen; halogen; —CN; —OH; —SH; —NO₂; or an unsubstituted or substituted moiety selected from lower alkyl, lower alkynyl, lower alkenyl, alkoxy, haloalkyl, aryl, and heteroaryl;

(d) R₆, R₇, R₈ and R₉ are each independently hydrogen; halogen; —CN; —OH; —SH; —NO₂; or an unsubstituted or substituted moiety selected from alkyl, alkenyl, alkynyl, alkoxy, allyloxy, aryl, heteroaryl, cycloalkyl, and heterocycloalkyl, wherein at least one of R₆, R₇, R₈ and R₉ is an unsubstituted or substituted moiety selected from aryl, heteroaryl, cycloalkyl, heterocycloalkyl, alkenyl, and alkynyl;

(e) R₁₀ is hydrogen; or an unsubstituted or substituted moiety selected from lower alkyl, lower alkenyl, lower alkynyl, aryl; heteroaryl, heterocycloalkyl, and cycloalkyl;

(f) Y is an unsubstituted or substituted moiety selected from alkyl, alkenyl, and alkynyl; and

(g) Z is a moiety selected from —OH, —NH₂, —SH, —SO₂OH, —S(O)H, —OC(O)NH₂, —S(O)₂NH₂, —NHC(O)H, C(O)NH₂, unsubstituted or substituted with one, two or three suitable substituents each independently selected from the group consisting of alkyl, haloalkyl, heteroalkyl, aryl, heteroaryl, cycloalkyl, and heterocycloalkyl;

wherein R₁ and Y may cyclize to form an unsubstituted or substituted cycloalkyl group or an unsubstituted or substituted heterocycloalkyl group;

or a pharmaceutically acceptable prodrug, pharmaceutically active metabolite, or pharmaceutically acceptable salt thereof.

In some embodiments, the substituted groups in R₂, R₃, R₄, R₅, R₆, R₇, R₈, R₉ and R₁₀ of Formula III are substituted with one, two or three suitable substituents each independently selected from the group consisting of: halogens, ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 0 to 6, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one, two or three suitable substituents each independently selected from the group consisting of halogens, ═O, —NO2, —CN, —OH, —SH, —(CH2)z-CN where z is an integer from 0 to 6, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyls, unsubstituted alkenyls, unsubstituted alkynyls, unsubstituted heteroalkyls, unsubstituted haloalkyls, unsubstituted aryls, unsubstituted cycloalkyls, unsubstituted heterocycloalkyls, and unsubstituted heteroaryls, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

In other embodiments, the substituted groups in R₂, R₃, R₄, R₅, R₆, R₇, R₈, R₉, and R₁₀ of Formula III are substituted with one, two or three suitable substituents each independently selected from the group consisting of: halogens, ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 0 to 6, ═NH, —OH, —C(O)H, —OC(O)H, —C(O)OH, —OC(O)OH, —C(NH)NH₂, —NHC(O)NH₂, —S(O)H, —NH₂, —C(O)NH₂, —OC(O)NH₂, —NHC(O)H, —NHC(O)OH, —C(S)H, and —SH groups unsubstituted or substituted with one, two or three suitable substituents each independently selected from the group consisting of halogens, ═O, —NO2, —CN, —OH, —SH, —(CH2)z-CN where z is an integer from 0 to 6, unsubstituted alkyls, unsubstituted alkenyls, unsubstituted alkynyls, unsubstituted heteroalkyls, unsubstituted haloalkyls, unsubstituted aryls, unsubstituted cycloalkyls, unsubstituted heterocycloalkyls, and unsubstituted heteroaryls.

The preparation of compounds of Formula I is disclosed in U.S. Pat. Nos. 3,843,681; 3,939,178; 3,974,179; 4,686,213; 4,748,252; 5,776,967; 5,824,699; 5,830,911; and 6,573,292; The disclosures of which are incorporated herein by reference except to the extent they are inconsistent with the present disclosure.

In a preferred embodiment of the invention refractory or resistant MM is treated with etodolac having the structure shown below (Formula IV)

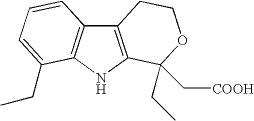

or a pharmaceutically acceptable salt thereof. Methods for the synthesis of etodolac are disclosed in U.S. Pat. Nos. 4,585,877 and 5,599,946, which are incorporated herein by reference. Etodolac is commercially available under the tradename Lodine®, (Wyeth-Ayerst Laboratories Division of American Home Products Corporation, Philadelphia, Pa.). Also included within the scope of this invention are the isomers of the compounds of Formula I resulting from the asymmetric centers contained therein. The resolution of racemic compounds of Formula (I) can be accomplished using conventional means, such as the formation of a diastereomeric salt with a optically active resolving amine; see, for example, “Stereochemistry of Carbon Compounds,” by E. L. Eliel (McGraw Hill, 1962); C. H. Lochmuller et al., J Chromatog., 113, 283 (1975); “Enantiomers, Racemates and Resolutions,” by J. Jacques, A. Collet, and S. H. Wilen, (Wiley-Interscience, New York, 1981); and S. H. Wilen, A. Collet, and J. Jacques, Tetrahedron, 33, 2725 (1977). The racemate of etodolac has been resolved by fractional crystallization of RS-etodolac using optically active 1-phenylethylamine and HPLC (U. Becker-Scharfenkamp et al., J. Chromatog., 621, 199 (1993)). B. M. Adger et al. (U.S. Pat. No. 5,811,558), disclosed the resolution of etodolac using glutamine and N(C1-C4 alkyl)-glutamine salts. U.S. Pat. No. 5,561,151 discloses the resolution of a mixture of the enantiomers of etodolac. All references regarding the resolution of the enantiomers of Formula I and etodolac are incorporated herein by reference.

Compounds of Formula (II) can be made as disclosed herein and as disclosed in U.S. Pat. No. 3,843,681, U.S. patent application Ser. No. 09/313,048, Ger. Pat. No. 2,226,340 (Amer. Home Products), R. R. Martel et al., Can. J. Pharmacol., 54, 245 (1976); Demerson et al., J. Med. Chem., 19, 391 (1976); PCT application Serial No. US/00/13410 and Rubin (U.S. Pat. No. 4,337,760).

Compounds of Formula (III) can be made as disclosed herein.

Compounds

Disclosed herein are compounds, as represented by Formulas I, II, III, and IV that possess COX inhibitory activity, β-catenin inhibitory activity, cyclin D1 inhibitory activity, and/or are cytotoxic to cancer cell lines, including multiple myeloma cell lines resistant to glucocorticoids, and other chemotherapeutic agents.

The compounds of Formulas I, II, III, and IV may exhibit the phenomenon of tautomerism. While Formulas I, II, III, IV cannot expressly depict all possible tautomeric forms, it is to be understood that Formulas I, II, III, IV are intended to represent any tautomeric form of the depicted compound and are not to be limited merely to a specific compound form depicted by the formula drawings.

The compounds of Formulas I, II, III, IV may have one or more asymmetric centers depending upon the nature of the various substituents on the molecule. As a consequence of these asymmetric centers, the compounds of Formulas I, II, III, IV may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form. An example, of a preferred enantiomer of the invention is R-etodolac.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. The compounds of the present invention can be used in a form that is at least 90% optically pure, that is, a form that contains at least 90% of a single isomer (80% enantiomeric excess (“e.e.”) or diastereomeric excess (“d.e.”)). In some cases, e.g., to reduce toxicity, the compounds can be used in a form that contains at least 95% (90% e.e. or d.e.), even more preferably at least 97.5% (95% e.e. or d.e.), and most preferably at least 99% (98% e.e. or d.e.) of a single isomer e.e. or d.e.

Additionally, Formula I, II, III, IV compounds are intended to cover solvated as well as unsolvated forms of the identified structures. For example, Formula III includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, II, III, and IV, the invention includes pharmaceutically acceptable prodrugs, pharmaceutically active metabolites, and pharmaceutically acceptable salts of such compounds and metabolites.

Prodrugs and active metabolites of a compound may be identified using routine techniques known in the art. See, e.g., Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, g-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. Such salts, as well as other compounds of the invention, can be used to produce medicines for treating refractory multiple myeloma.

In the case of agents that are solids, it is understood by those skilled in the art that the inventive compounds and salts may exist in different crystal or polymorphic forms, all of which are intended to be within the scope of the present invention and specified formulas.

Therapeutically effective amounts of the agents of the invention may be used to treat or prevent diseases and/or conditions mediated by modulation or regulation of β-catenin, COX, cyclin D, Mcl-1s and PPAR.

The amount of a given agent that will correspond to a therapeutically effective amount will vary depending upon factors such as the particular compound, disease condition and its severity, the identity (e.g., weight) of the subject in need of treatment, but can nevertheless be routinely determined by one skilled in the art.

The active agents of the invention may be formulated into pharmaceutical compositions as described below. Pharmaceutical compositions of this invention comprise an effective, modulating, regulating, or inhibiting amount of a compound of Formula I, II, III, IV and an inert, pharmaceutically acceptable carrier or diluent. In one embodiment of the pharmaceutical compositions, efficacious levels of the inventive agents are provided so as to provide therapeutic benefits involving modulation of β-catenin, COX, Mcl-1s, PPAR, and/or Cyclin D. These compositions are prepared in unit-dosage form appropriate for the mode of administration, e.g., parenteral or oral administration.

An inventive agent can be administered in conventional dosage form prepared by combining a therapeutically effective amount of an agent (e.g., a compound of Formulas I, II, III, or IV) as an active ingredient with appropriate pharmaceutical carriers or diluents according to conventional procedures. These procedures may involve mixing, granulating and compressing or dissolving the ingredients as appropriate to the desired preparation.

The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 0-60% of the total volume. In an exemplary embodiment, a compound of Formulas I, II, III, or IV is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease and/or condition being treated. Optimal dosages for a given set of conditions can be ascertained by those skilled in the art using conventional dosage-determination tests in view of the experimental data for an agent. For oral administration, an exemplary daily dose generally employed is from about 0.001 to about 3000 mg/kg of body weight, with courses of treatment repeated at appropriate intervals. In some embodiments, the daily dose is from about 1 to 3000 mg/kg of body weight.

Typical daily doses in a patient may be anywhere between about 200 mg to about 3000 mg, given once or twice daily, e.g., 3000 mg can be given twice daily for a total dose of 6000 mg. In one embodiment, the dose is between about 1000 mg to about 3000 mg. In another embodiment, the dose is between about 1500 mg to about 2800 mg. In other embodiments, the dose is between about 2000 mg to about 3000 mg.

Plasma concentrations in the subjects may be between about 100 μM to about 1000 μM. In some embodiments, the plasma concentration may be between about 200 μM to about 800 μM. In other embodiments, the concentration is about 300 μM to about 600 μM. In still other embodiments the plasma concentration may be between about 400 to about 800 μM. Administration of prodrugs is typically dosed at weight levels, which are chemically equivalent to the weight levels of the fully active form.

The compositions described herein may be manufactured using techniques generally known for preparing pharmaceutical compositions, e.g., by conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds disclosed herein may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The administration of the compounds disclosed herein may be for either prevention or treatment purposes. When used for the treatment and/or prevention of neoplasia, or for the treatment of diseases treatable by inhibiting COX, the methods and compositions described herein may be used alone or in conjunction with additional therapies known to those skilled in the art, in particular therapeutic regimens and agents useful for treating refractory or resistant multiple myeloma. By way of example, the compounds described herein may be administered alone or in conjunction with other antineoplastic agents, glucocorticoids or other growth inhibiting agents or other drugs or nutrients. Alternatively, the compounds of the invention may be used in conjunction with other treatments, such as radiation therapy (for example, external beam radiation,), allogeneic or autologous peripheral blood stem cell or bone marrow transplantation, immunotherapy, and/or treatments intended to improve or restore hematopoietic function (e.g., the administration of erythropoietin), kidney function, and the like. Other treatment regimens may also be combined, including nutritional and/or naturopathic treatments.

There are large numbers of antineoplastic agents available in commercial use, in clinical evaluation and in pre-clinical development, which could be selected for treatment of neoplasia by combination drug chemotherapy. Such antineoplastic agents fall into several major categories, namely, antibiotic-type agents, alkylating agents, antimetabolite agents, hormonal agents, including glucocorticoids such as prednisone and dexamethasone, immunological agents, interferon-type agents and a category of miscellaneous agents. Alternatively, other anti-neoplastic agents, such as metallomatrix proteases (MMP), SOD mimics or alphaᵥ beta₃ inhibitors may be used.

One family of antineoplastic agents which may be used in combination with the compounds of the inventions consists of antimetabolite-type antineoplastic agents. Suitable antimetabolite antineoplastic agents may be selected from the group consisting of alanosine, AG2037 (Pfizer), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT and uricytin.

A second family of antineoplastic agents which may be used in combination with the compounds of the invention consists of alkylating-type antineoplastic agents. Suitable alkylating-type antineoplastic agents may be selected from the group consisting of Shionogi 254-S, aldo-phosphamide analogues, altretamine, anaxirone, Boehringer Mannheim BBR-2207, bendamustine, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, melphalan, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromustine, Tanabe Seiyaku TA-077, tauromustine, temozolomide, teroxirone, tetraplatin and trimelamol.

Another family of antineoplastic agents which may be used in combination with the compounds disclosed herein consists of antibiotic-type antineoplastic agents. Suitable antibiotic-type antineoplastic agents may be selected from the group consisting of Taiho 4181-A, aclarubicin, actinomycin D, actinoplanone, alanosine, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SR1 International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with the compounds of the invention include a miscellaneous family of antineoplastic agents selected from the group consisting of alpha-carotene, alpha-difluoromethyl-arginine, acitretin, arsenic trioxide, Avastin® (bevacizumab), Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristo-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, epothionesTsumura EPMTC, erbitux, ergotamine, erlotnib, etoposide, etretinate, fenretinide, Fujisawa FR-57704, gallium nitrate, genkwadaphnin, Glivec® (imatnib), Chugai GLA-43, Glaxo GR-63178, gefitinib, grifolan NMF-5N, hexadecylphosphocholine, Green Cross HO-221, homoharringtonine, hydroxyurea, BTG ICRF-187, indanocine, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, mefloquine, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, Rituxan® (and other anti CD20 antibodies, e.g. Bexxar®, Zevalin®), SmithKline SK&F-104864, statins (Lipitor® etc.), Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Thalidomide, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534, zometa.

Preferred antineoplastic agents for combinations of the present invention include vincristine, doxorubicin, dexamethasone, thalidomide, thalidomide derivatives, 2ME2, Neovastat, R 11 5777 (Janssan Pharmaceuticals), arsenic trioxide, bortezomib, tamoxifen, G3139 (antisense), and SU5416. A preferred class of compounds includes proteasome inhibitors.

Examples of radioprotective agents which may be used in the combination chemotherapy of this invention are AD-5, adchnon, amifostine analogues, detox, dimesna, 1-102, MM-159, N-acylated-dehydroalanines, TGF-Genentech, tiprotimod, amifostine, WR-151327, FUT-187, ketoprofen transdermal, nabumetone, superoxide dismutase (Chiron) and superoxide dismutase Enzon.

Methods for preparation of the antineoplastic agents described above may be found in the literature. Methods for preparation of doxorubicin, for example, are described in U.S. Pat. Nos. 3,590,028 and 4,012,448. Methods for preparing metallomatrix protease inhibitors are described in EP 780386. Methods for preparing SOD mimics are described in EP 524,101. Methods for preparing .alphaᵥ .beta₃ inhibitors are described in WO97/08174.

Preparation of Compounds of the Invention

Compounds of the present invention may be synthesized using standard synthetic techniques known to those of skill in the art or using methods known in the art in combination with methods described herein. See, e.g., March, ADVANCED ORGANIC CHEMISTRY 4^{th} Ed., (Wiley 1992); Carey and Sundberg, ADVANCED ORGANIC CHEMISTRY 3^{rd} Ed., Vols. A and B (Plenum 1992), and Green and Wuts, PROTECTIVE GROUPS IN ORGANIC SYNTHESIS 2^{nd} Ed. (Wiley 1991). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

Selected examples of covalent linkages and precursor functional groups which yield them are given in the Table entitled “Examples of Covalent Linkages and Precursors Thereof.” Precursor functional groups are shown as electrophilic groups and nucleophilic groups. The functional group on the organic substance may be attached directly, or attached via any useful spacer or linker as defined below.

### Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| Hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, e.g, oxygen or nitrogen.

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

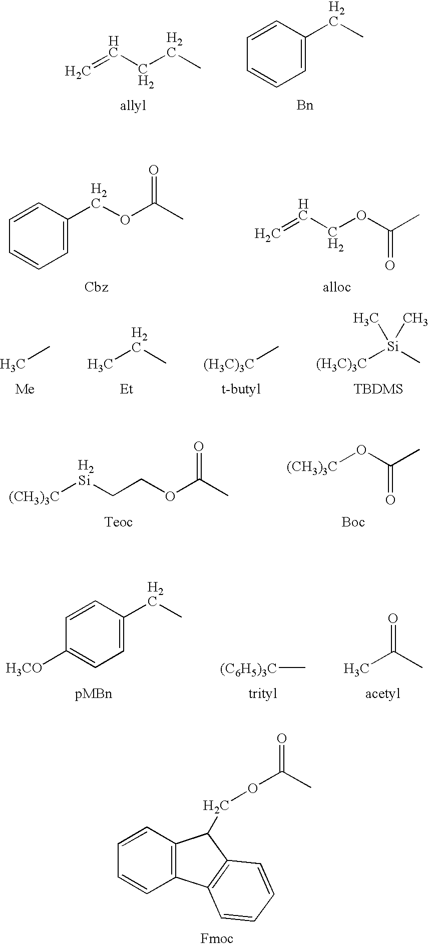

Other protecting groups are described in Greene and Wuts, Protective Groups in Organic Synthesis, 3rd Ed., John Wiley & Sons, New York, N.Y., 1999, which is incorporated herein by reference in its entirety.

In various embodiments, the compounds of the present invention can be prepared according to the following reaction schemes and examples, or modifications thereof. Starting materials can be purchased or made from procedures known in the art or as illustrated. In these reactions, one skilled in the art can make use of variations that are not described in greater detail. Other methods for preparing compounds of the invention will be readily apparent to the person of ordinary skill in the art in light of the following reaction schemes and examples. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention. Unless otherwise indicated, the variables are as defined above.

The abbreviations employed throughout the application have the following meaning unless otherwise indicated: EtOH: ethyl alcohol; NH₂OH.HCl: hydroxylamine; CCl₃CH(OH)₂: chloral hydrate; H₂SO₄: sulfuric acid; LiBH₄: lithium borohydride; ClCOCOCl: oxalyl chloride; HCl: Hydrochloric acid; NaOH: sodium hydroxide; BF₃.Et₂O: boron trifluoride etherate; CH₂Cl₂: dichloromethane; [R]: partial reduction.

General Scheme 1A shows the preparation of pyranoindol-1-yl alcohols from starting material 1.

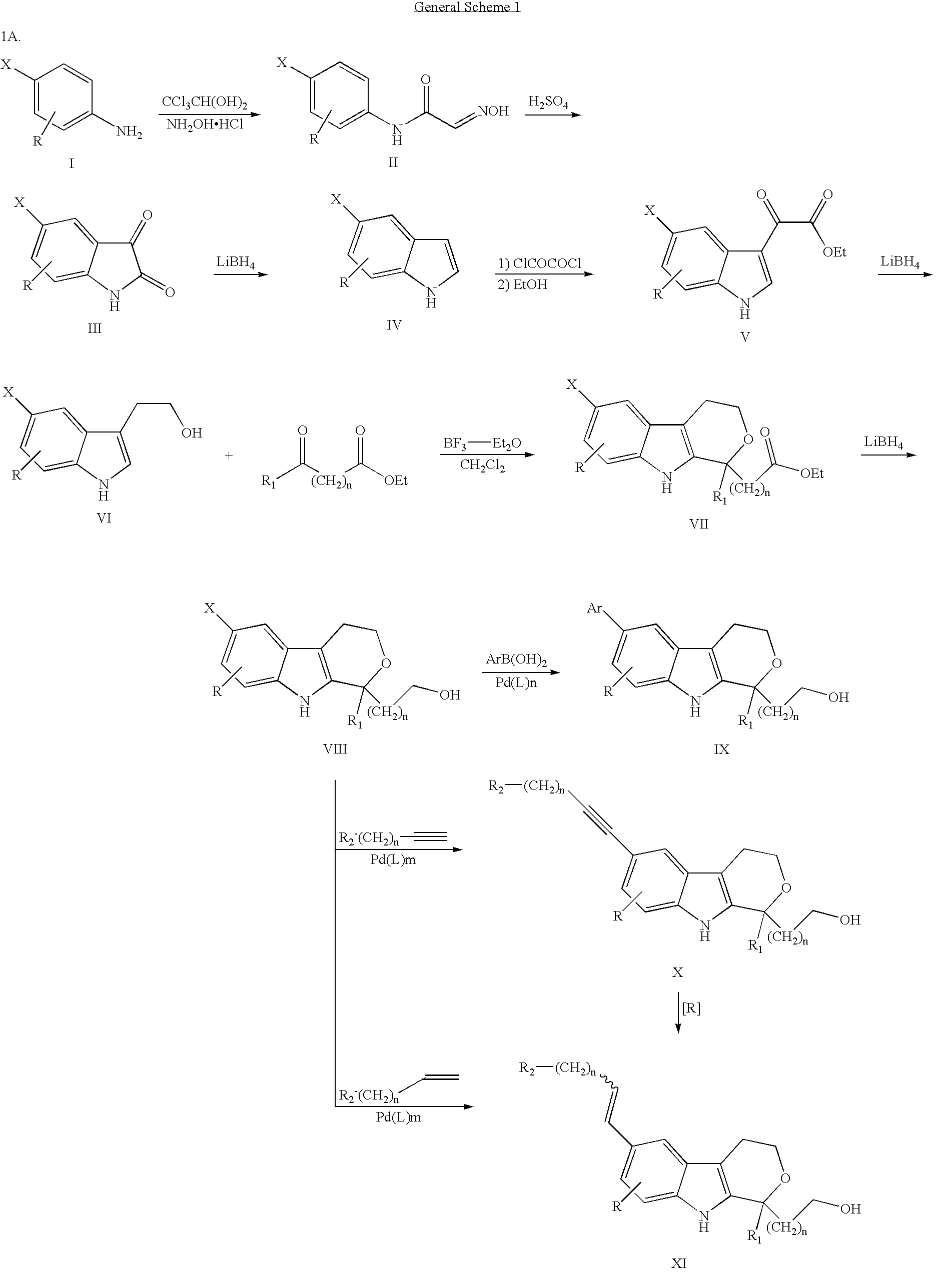

In General Scheme 1, 1,3,4,9-tetrahydro-pyrano[3,4-b]indole of this invention may be prepared by techniques well known to those skilled in the art of organic synthesis. The substituted tryptophols (VI) may be prepared by the appropriate segment of the pathway illustrated in General Scheme 1A, starting with an aniline (I), an isatin (III), or an indole (IV). The suitable starting materials are commercially available anilines with the desired R or may be readily prepared. The aniline may be converted into a corresponding isatin (III) by treatment of aniline with chloral hydrate and hydroxylamine, followed by heating with sulfuric acid. The indole (IV) may be obtained by reduction of isatin with lithium borohydride or other reducing agents. The tryptophol (VI) may be prepared by acylation at 3-position of indole (IV) with a suitable reagent, e.g., oxalyl chloride, followed by reduction of glyoxylate (V) with lithium borohydride. The substituted tryptopholes (VI) may be condensed with an appropriate ketone or aldehyde, in the presence of an acid catalyst, to provide 1,3,4,9-tetradydro-pyrano[3,4-b]indole (VII). After the ester (VII) is reduced by an appropriate reducing reagent, e.g., lithium borohydride, the title compounds (IX) may be prepared from (VIII) by displacement of the halogen with an appropriately activated Ar moiety. For example, in the presence of an appropriate Pd(L)m catalyst, Ar-boronic acids may be coupled via a Suzuki reaction to give the title compounds (IX). Compounds (X) and (XI) may be prepared, via Heck reaction, from suitable alkyne and alkene precursors in the presence of an appropriate Pd(L)m catalyst. The cis isomer of (XI) may also be prepared by partial reduction of (X) by hydrogenation over palladium on activated carbon that has been treated with quinoline.

General Scheme 1B shows the preparation of pyranoindol-1-yl alkylsulfonamides from starting material 6.

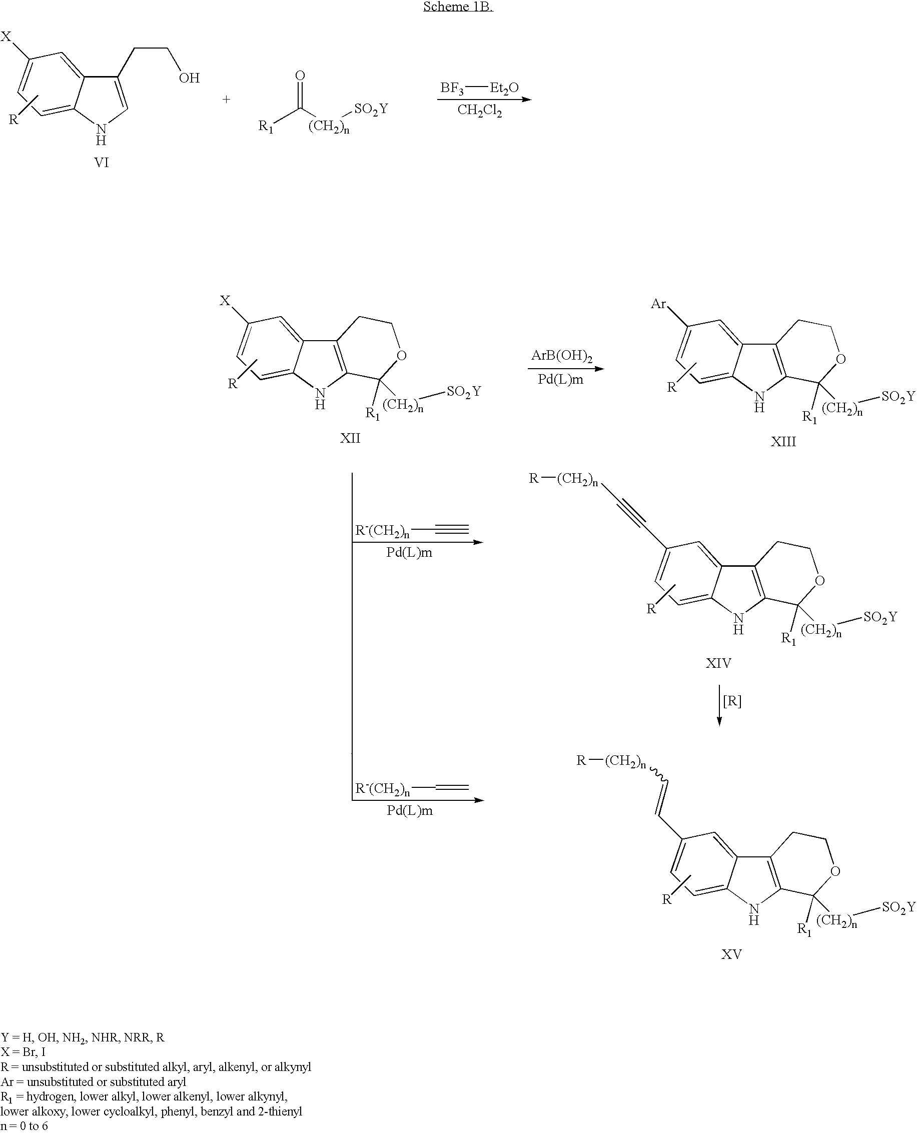

Scheme 1B illustrates syntheses of the title compounds (XIII), (XIV), or (XV) wherein —(CH₂)nSO₂Y is substituted at 1-position of 1,3,4,9-tetradydro-pyrano[3,4-b]indole. The compounds (XIII) may be prepared by condensation of tryptophols (VI) with an appropriate ketone or aldehyde bearing —SO₂Y in the presence of a suitable acid, followed by coupling reactions, which may be via Suzuki reaction with a suitable activated Ar moiety in the presence of an appropriate Pd(L)m catalyst. Analogously, compounds (XIV) and (XV) may be prepared, via Heck reaction, from suitable alkyne and alkene in the presence of an appropriate Pd(L)m catalyst.

General Scheme 2 illustrates the additional embodiment wherein R₁₀ is lower alkyl, lower alkenyl, lower alkynyl, or aryl. The nitrogen of compound (VII) may be alkylated with an appropriate alkyl halide in the presence of a suitable base. After the ester is reduced to the alcohol (XVII) by a suitable reducing reagent, e.g., lithium borohydride, the title compounds (XVIII), (XIX), or (XX) may be prepared by coupling reactions, e.g., Suzuki reaction or Heck reaction.

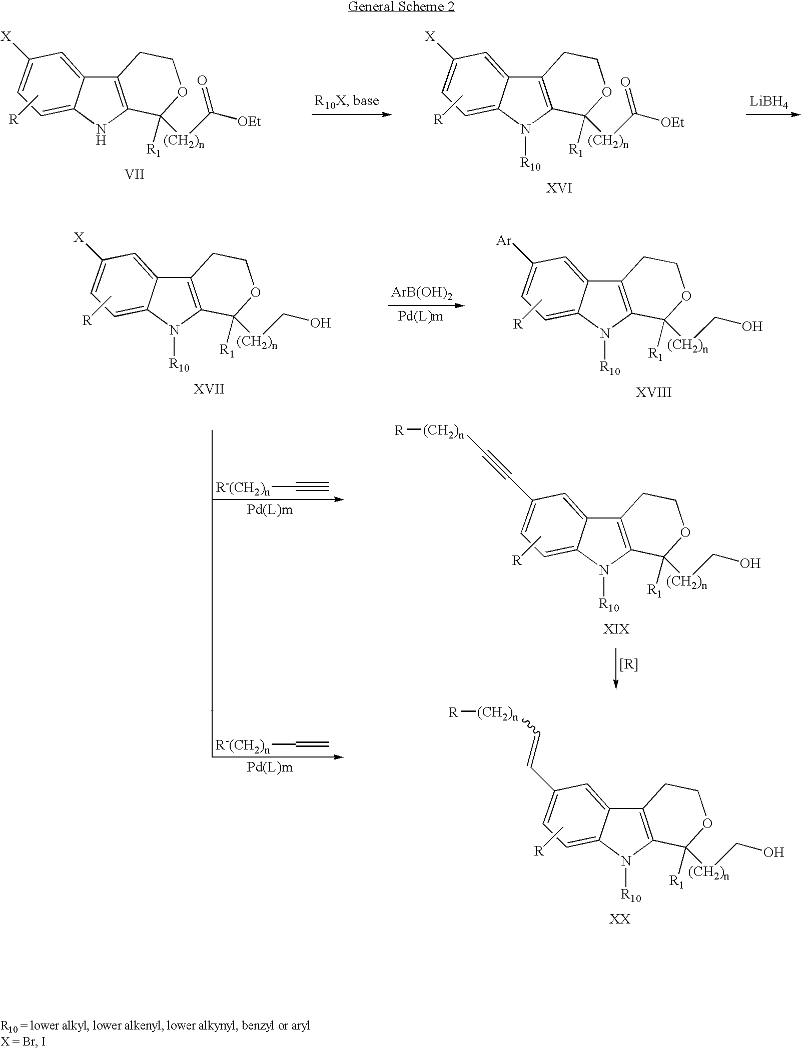

General Scheme 3 illustrates the synthesis of compounds where R₇ is substituted, R₉ is an isopropyl group, R₁ is ethyl, and Y-Z is ethylalcohol.

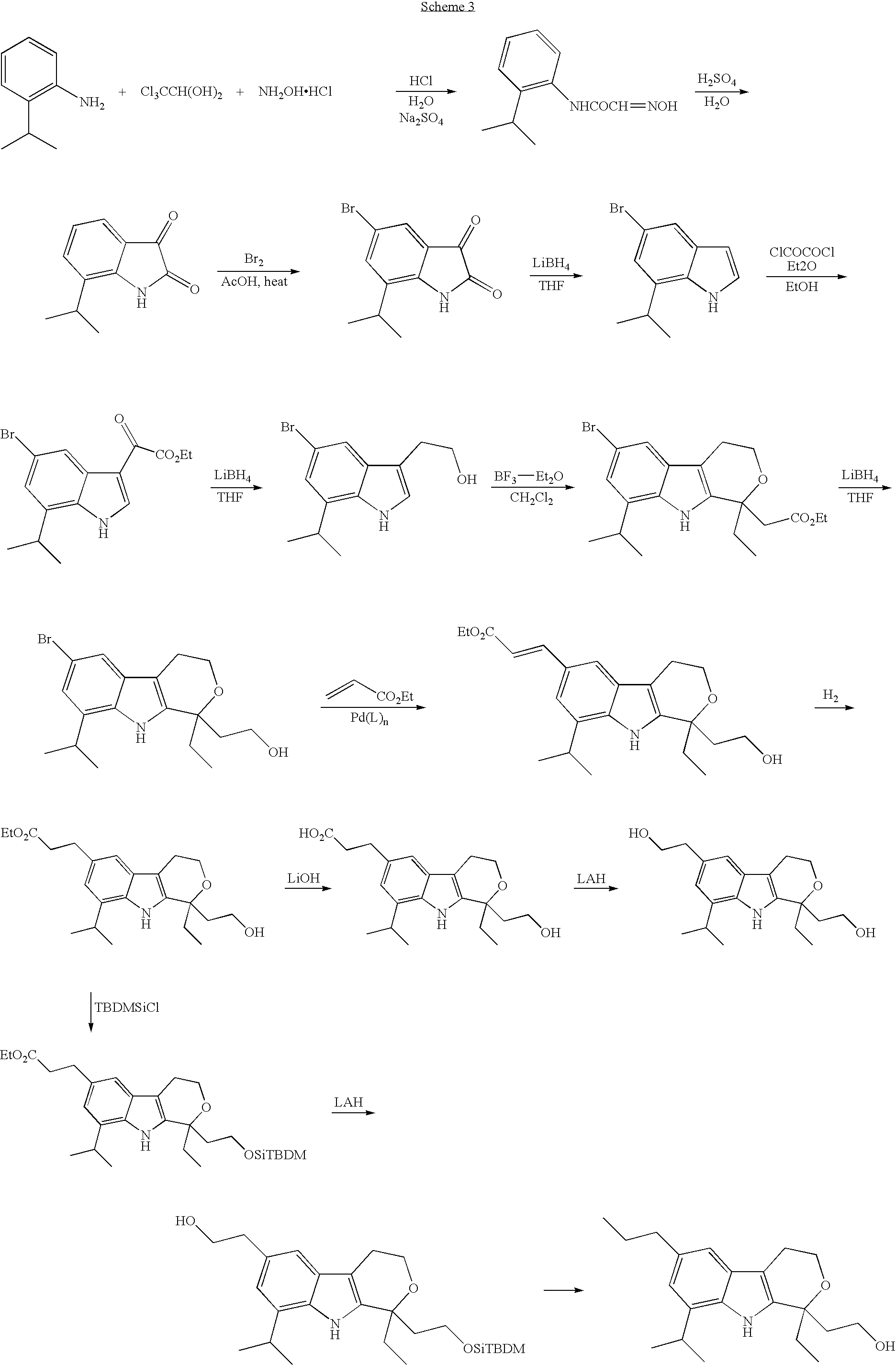

General Scheme 4 illustrates the synthesis of pyranoindol-1-yl alcohols.

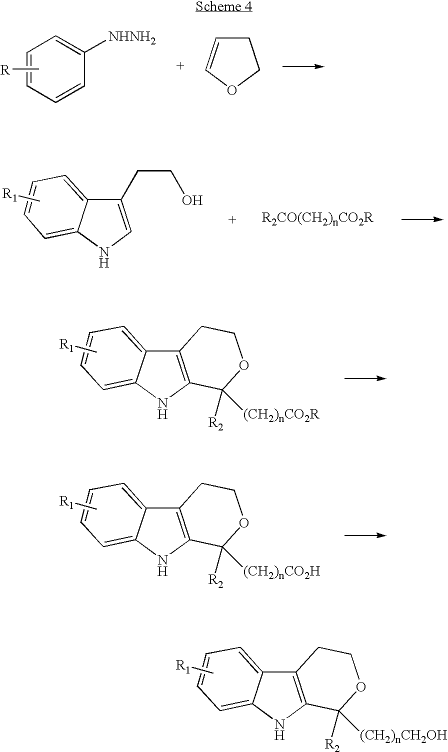

### EXAMPLES

### Example 1

### Synthesis of Compounds

### COMPOUND 1: 2-(1,8-DIETHYL-6-PHENYL-1,3,4,9-TETRAHYDRO-PYRANO[3,4-B]INDOL-1-YL)-ETHANOL

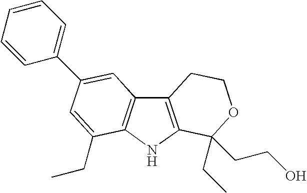

### 1.A. Synthesis of N-(4-Bromo-2-ethyl-phenyl)-2-hydroximino-acetoamide

To a suspension of 4-bromo-2-ethylaniline (50.0 g, 250 mmol) in water (1000 mL) was added concentrated hydrochloric acid (25 mL), sodium sulfate (220 g), and hydroxylamine hydrochloride (56.25 g), followed by addition of chloral hydrate (44.0 g). The reaction mixture was heated to 90° C. using an oil bath for 1 hour. After cooling down to room temperature, it was extracted with ethyl acetate. Extract was dried over magnesium sulfate and concentrated under reduced pressure to give the title compound (31.1 g, 46% yield). ¹H NMR (DMSO-d₆) δ 12.24 (s, 1H), 9.56 (s, 1H), 7.68 (s, 1H), 7.41 (m, 3H), 2.58 (q, 2H), 1.11 (t, 3H).

### 1.B. Synthesis of 5-Bromo-7-ethyl-1H-indole-2,3-dione

To a solution of sulfuric acid (100 mL) and water (10 mL) at 80° C. (oil bath) was added N-(4-bromo-2-ethyl-phenyl)-2-hydroximino-acetoamide (61.0 g, 225 mmol) in small portions over 20 minutes. The reaction mixture was heated at 80° C. (oil bath) for 15 minutes. After cooling to room temperature, ice-water (500 mL) was added and the mixture was extracted with ethyl acetate. Extracts were washed with saturated sodium bicarbonate solution, dried over magnesium sulfate, and concentrated under reduced pressure to give the title compound (42.3 g, 74% yield). ¹H NMR (DMSO-d₆) δ 8.87 (s, 1H), 7.75 (d, 1H), 7.71 (d, 1H), 2.75 (q, 2H), 1.44 (t, 3H).

### 1.C. Synthesis of (5-Bromo-7-ethyl-1H-indol-3-yl)-oxo-acetic acid ethyl ester

To a solution of 5-bromo-7-ethyl-1H-indole-2,3-dione (36.g, 144 mmol) in tetrahydrofuran (120 mL) at room temperature was dropped a 2.0 M solution of lithium borohydride in tetrahydrofuran. The reaction mixture was stirred at 90° C. (oil bath) for 5 hours. After cooling down to room temperature, it was quenched with 5% hydrochloric acid solution until the excess lithium borohydride was destroyed. To the mixture was added saturated sodium bicarbonate solution (300 mL) and extracted with ethyl acetate. Extracts were dried over magnesium sulfate and concentrated under reduced pressure to give the crude product of 5-bromo-7-ethyl-1H-indole, which went to next reaction without further purification.

To a solution of 5-bromo-7-ethyl-1H-indole in ethyl ether (400 mL) at room temperature under nitrogen was added a 2.0 M solution of oxalyl chloride in dichloromethane. After the reaction mixture was stirred at room temperature for 6 hours, the solvents were removed under reduce pressure. To the residue was added ethyl alcohol (400 mL) and stirred at room temperature overnight. After ethyl alcohol was removed under reduce pressure, to the residue was added saturated sodium bicarbonate solution (300 mL) and extracted with ethyl acetate. Extract was dried over magnesium sulfate and concentrated under reduced pressure. The crude product was purified by column chromatography (silica gel, 30-50% ethyl acetate/hexane) to give the title compound (14.5 g, 31% yield). ES-MS (m/z) 324 [M+1]⁺, 322 [M−1]⁻.

### 1.D. Synthesis of (6-Bromo-1,8-diethyl-1,3,4,9-tetrahydro-pyrano[3,4-b]indol-1-yl)-acetic acid ethyl ester

To a solution of (5-bromo-7-ethyl-1H-indol-3-yl)-oxo-acetic acid ethyl ester (1.55 g, 4.8 mmol) in tetrahydrofuran at room temperature under nitrogen was dropped a 2.0 M solution of lithium borohydride in tetrahydrofuran. The reaction mixture was heated at 90° C. oil bath for 5 hours. After cooling to room temperature, it was quenched with 5% hydrochloric acid solution until the excess lithium borohydride was destroyed. To the mixture was added saturated sodium bicarbonate solution and extracted with ethyl acetate. Extracts were dried over magnesium sulfate and concentrated under reduced pressure to give the crude product of 2-(5-bromo-7-ethyl-1H-indol-3-yl)-ethanol, which went to the next reaction without further purification.

To a solution of 2-(5-bromo-7-ethyl-1H-indol-3-yl)-ethanol in dichloromethane at room temperature under nitrogen was added boron trifluoride diethyl etherate (0.809 g, 5.7 mmol), followed by ethyl propionylacetate (1.038 g, 7.2 mmol). The reaction mixture was stirred at room temperature for 5 hours. It was quenched with saturated sodium bicarbonate solution and extracted with dichloromethane. The extract was dried over magnesium sulfate and concentrated under reduced pressure. The crude product was purified by column chromatography (silica gel, 15-20% ethyl acetate/hexane) to give the title compound (0.994 g, 53% yield). ES-MS (m/z) 394 [M+1]⁺, 392 [M−1]⁻.

### 1.E. Synthesis of 2-(6-Bromo-1,8-diethyl-1,3,4,9-tetrahydro-pyrano[3,4-b]indol-1-yl)-ethanol

To a solution of (6-bromo-1,8-diethyl-1,3,4,9-tetrahydro-pyrano[3,4-b]indol-1-yl)-acetic acid ethyl ester (5.2 g, 13.2 mmol) in tetrhydrofuran at room temperature under nitrogen was dropped a 2.0 M solution of lithium borohydride in tetrahydrofuran. The reaction mixture was heated at 90° C. (oil bath) for 5 hours. After cooling to room temperature, it was quenched with 5% hydrochloric acid solution until the excess lithium borohydride was destroyed. Water was added and the mixture extracted with ethyl acetate. Extracts were dried over magnesium sulfate and concentrated under reduced pressure. The crude product was purified by column chromatography (silica gel, 50% ethyl acetate/hexane) to give the title compound (3.80 g, 82% yield). 1H NMR (CDCl₃) δ 8.07 (s, 1H), 7.64 (d, 1H), 7.26 (d, 1H), 4.17 (m, 2H), 3.86 (m, 2H), 2.94 (m, 3H), 2.87 (dt, 1H), 2.76 (t, br, 1H), 2.36 (m, 1H), 2.24 (m, 1H), 2.13 (m, 2H), 1.49 (t, 3H), 1.08 (t, 3H). ES-MS (m/z) 352 [M+1]⁺, 350 [M−1]⁻.

### 1.F Synthesis of 2-(1,8-Diethyl-6-phenyl-1,3,4,9-tetrahydro-pyrano[3,4-b]indol-1-yl)-ethanol

To a solution of 2-(6-bromo-1,8-diethyl-1,3,4,9-tetrahydro-pyrano[3,4-b]indol-1-yl)-ethanol (3.8 g, 10.8 mmol) in ethylene glycol dimethyl ether (50 mL) was added potassium phosphate (6.37 g, 30 mmol), phenylboronic acid (1.83 g, 15 mmol), and [1,1′-bis(diphenylphosphino)ferrocene]dichloropalladium (II) complex with dichloromethane. The reaction mixture was heated at 90° C. (oil bath) overnight. It was quenched with water and extracted with ethyl acetate. Extracts were dried over magnesium sulfate and concentrated under reduced pressure. The crude product was purified by column chromatography (silica gel, 50% ethyl acetate/hexane, followed by Sephadex LH-20, 50% chloroform/hexane) to give the title compound (0.75 g, 20% yield). ¹H NMR (CDCl₃) δ 7.77 (s, 1H), 7.66 (d, 1H), 7.63 (m, 1H), 7.56 (d, 1H), 7.44 (m, 3H), 7.32 (m, 1H), 4.06 (m, 2H), 3.72 (m, 3H), 2.91 (m, 3H), 2.81 (dt, 1H), 2.65 (dd, 1H), 2.20 (m, 1H), 2.07 (m, 2H), 1.40 (t, 3H), 0.95 (t, 3H). ES-MS (m/z) 348 [M−1]−.

### COMPOUND 2: 2-[1,8-DIETHYL-6-(4-METHOXY-PHENYL)-1,3,4,9-TETRAHYDRO-PYRANO[3,4-B]INDOL-1-YL]-ETHANOL

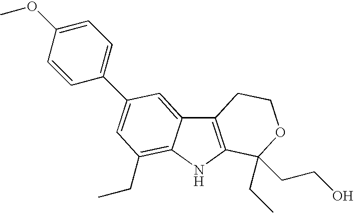

The title compound is prepared in a manner analogous to Example 1, except using 4-methoxyphenylboronic acid in step 1.F.

### COMPOUND 3: 2-[1,8-DIETHYL-6-(3-TRIFLUOROMETHOXY-PHENYL)-1,3,4,9-TETRAHYDRO-PYRANO[3,4-B]INDOL-1-YL]-ETHANOL

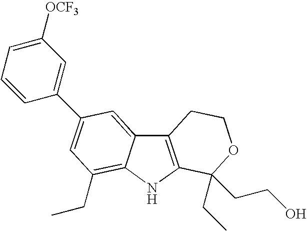

The title compound is prepared in a manner analogous to Example 1, except using 3-trifluoromethoxyphenylboronic acid in step 1.F.

### COMPOUND 4: 2-[1,8-DIETHYL-6-(2-TRIFLUOROMETHYL-PHENYL)-1,3,4,9-TETRAHYDRO-PYRANO[3,4-B]INDOL-1-YL]-ETHANOL

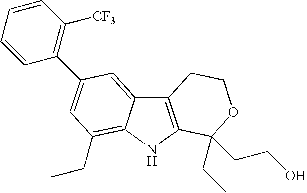

The title compound is prepared as described in Example 1, except using 2-trifluoromethylphenylboronic acid in step 1.F.

### COMPOUND 5: 2-[6-(2,4-DIFLUORO-PHENYL)-1,8-DIETHYL-1,3,4,9-TETRAHYDRO-PYRANO[3,4-B]INDOL-1-YL]-ETHANOL

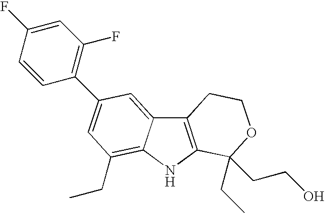

The title compound is prepared in a manner analogous to Example 1, except using 2,4-difluorophenylboronic acid in step 1.F.

### COMPOUND 6: 2-(1,8-DIETHYL-6-PYRIDIN-4-YL-1,3,4,9-TETRAHYDRO-PYRANO[3,4-B]INDOL-1-YL)-ETHANOL

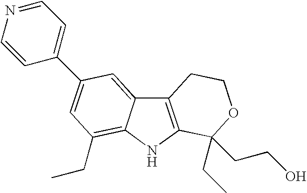

The title compound is prepared in a manner analogous to Example 1, except using pyridine-4-boronic acid in step 1.F.

### COMPOUND 8: 2-[6-(3-AMINO-PHENYL)-1,8-DIETHYL-1,3,4,9-TETPAHYDRO-PYRANO[3,4-B]INDOL-1-YL]-ETHANOL

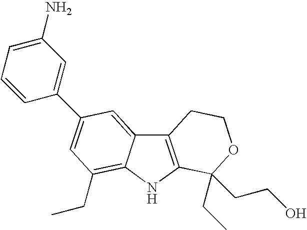

The title compound is prepared in a manner analogous to Example 1, except using 3-aminophenylboronic acid in step 1.F.

### COMPOUND 10: 2-[6-(3,4-DIFLUORO-PHENYL)-1,8-DIETHYL-1,3,4,9-TETRAHYDRO-PYRANO[3,4-B]INDOL-1-YL]-ETHANOL

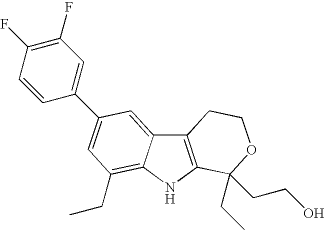

The title compound is prepared in a manner analogous to Example 1, except using 3,4-difluorophenylboronic acid in step 1.F.

### COMPOUND 11: 2-[6-(5-CHLORO-THIOPHEN-2-YL)-1,8-DIETHYL-1,3,4,9-TETRAHYDRO-PYRANO[3,4-B]INDOL-1-YL]-ETHANOL

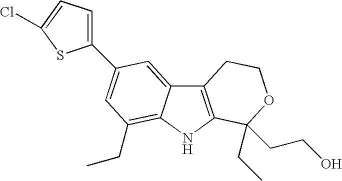

The title compound is prepared in a manner analogous to Example 1, except using 5-chloro-2-thiopheneboronic acid in step 1.F.

### COMPOUND 12: 2-(1-ETHYL-6-ISOPROPYL-8-PHENYL-1,3,4,9-TETRAHYDRO-PYRANO[3,4-B]INDOL-1-YL)-ETHANOL

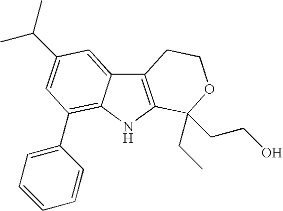

### 12.A. Synthesis of N-(2-Bromo-4-isopropyl-phenyl)-2-hydroximino-acetoamide

The title compound is prepared in a manner analogous to Example 1, except using 2-bromo 4-aminoaniline in step 1.A.

### 12.B. Synthesis of 7-Bromo-5-isopropyl-1H-indole-2,3-dione

The title compound is prepared in a manner analogous to Example 1, except using N-(2-bromo-4-isopropyl-phenyl)-2-hydroximino-acetoamide in step 1.B.

### 12.C. Synthesis of (7-Bromo-5-isopropyl-1H-indol-3-yl)-oxo-acetic acid ethyl ester

The title compound is prepared in a manner analogous to Example 1, except using 7-bromo-5-isopropyl-1H-indole-2,3-dione in step 1.C.

### 12.D. Synthesis of (8-Bromo-1-ethyl-6-isopropyl-1,3,4,9-tetrahydro-pyrano[3,4-b]indol-1-yl)-acetic acid ethyl ester

The title compound is prepared in a manner analogous to Example 1, except using (7-bromo-5-isopropyl-1H-indol-3-yl)-oxo-acetic acid ethyl ester in step 1.D.

### 12.E. Synthesis of 2-(8-Bromo-1-ethyl-6-isopropyl-1,3,4,9-tetrahydro-pyrano[3,4-b]indol-1-yl)-ethanol

The title compound is prepared in a manner analogous to Example 1, except using (8-bromo-1-ethyl-6-isopropyl-1,3,4,9-tetrahydro-pyrano[3,4-b]indol-1-yl)-acetic acid ethyl ester in step 1.E.

### 12. F. Synthesis of 2-(1-Ethyl-6-isopropyl-8-phenyl-1,3,4,9-tetrahydro-pyrano[3,4-b]indol-1-yl)-ethanol

The title compound is prepared in a manner analogous to Example 1, except using 2-(8-bromo-1-ethyl-6-isopropyl-1,3,4,9-tetrahydro-pyrano[3,4-b]indol-1-yl)-ethanol in step 1.F.

### COMPOUND 13: 2-[8-(3-CYANO-PHENYL)-1-ETHYL-6-ISOPROPYL-1,3,4,9-TETRAHYDRO-PYRANO[3,4-B]INDOL-1-YL]-ETHANOL

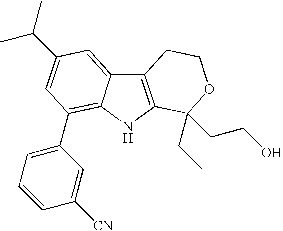

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 3-cyanophenylboronic acid in step 1.F.

### COMPOUND 14: 2-[8-(5-BROMO-2-METHOXY-PHENYL)-1-ETHYL-6-ISOPROPYL-1,3,4,9-TETRAHYDRO-PYRANO[3,4-B]INDOL-1-YL]-ETHANOL

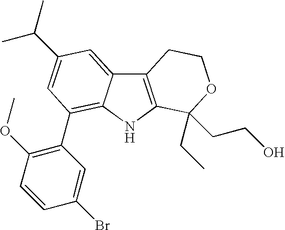

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 2-bromo-3-methoxyphenylboronic acid in step 1.F.

### COMPOUND 15: 2-[1-ETHYL-8-(2-FLUORO-BIPHENYL-4-YL)-6-ISOPROPYL-1,3,4,9-TETRAHYDRO-PYRANO[3,4-B]INDOL-1-YL]-ETHANOL

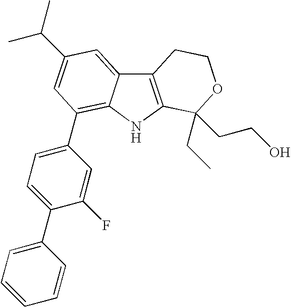

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 2-fluorobiphenyl-4-boronic acid in step 1.F.

### COMPOUND 16: 4-[1-ETHYL-1-(2-HYDROXY-ETHYL)-6-ISOPROPYL-1,3,4,9-TETRAHYDRO-PYRANO[3,4-B]INDOL-8-YL]-BENZOIC ACID

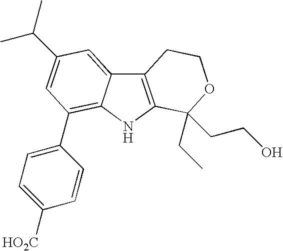

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 4-carboxylphenylboronic acid in step 1.F.

### COMPOUND 17: 3-[1-ETHYL-1-(2-HYDROXY-ETHYL)-6-ISOPROPYL-1,3,4,9-TETRAHYDRO-PYRANO[3,4-B]INDOL-8-YL]-BENZALDEHYDE

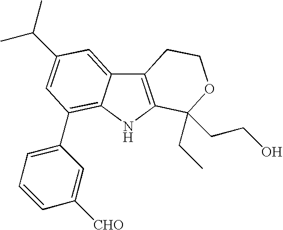

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 3-formylphenylboronic acid in step 1.F.

### COMPOUND 18: 2-[8-(3,5-DIMETHYL-PHENYL)-1-ETHYL-6-ISOPROPYL-1,3,4,9-TETRAHYDRO-PYRANO[3,4-B]INDOL-1-YL]-ETHANOL

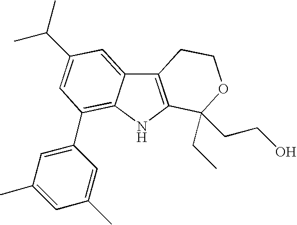

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 3,5-dimethylphenylboronic acid in step 1.F.

### COMPOUND 19: 2-(8-DIBENZOFURAN-3-YL-1-ETHYL-6-ISOPROPYL-1,3,4,9-TETRAHYDRO-PYRANO[3,4-B]INDOL-1-YL)-ETHANOL

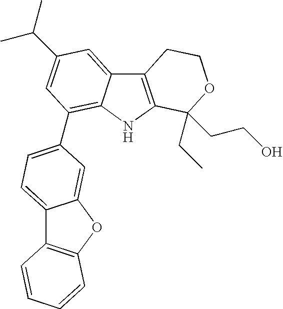

The title compound is prepared in a manner analogous to Example 1, except using 2-(8-bromo-1-ethyl-6-isopropyl-1,3,4,9-tetrahydro-pyrano[3,4-b]indol-1-yl)-ethanol and 4-dibenzofuranboronic acid in step 1.F.

### COMPOUND 20: 2-(1-ETHYL-6-ISOPROPYL-8-STYRYL-1,3,4,9-TETRAHYDRO-PYRANO[3,4-B]INDOL-1-YL)-ETHANOL

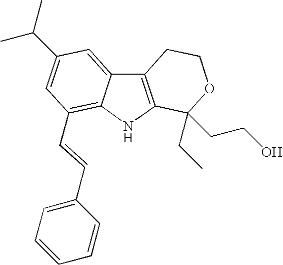

### 20.A. Synthesis of 2-(1-Ethyl-6-isopropyl-8-styryl-1,3,4,9-tetrahydro-pyrano[3,4-b]indol-1-yl)-ethanol

The title compound is prepared according to the following procedure. To solution of 2-(8-bromo-1-ethyl-6-isopropyl-1,3,4,9-tetrahydro-pyrano[3,4-b]indol-1-yl)-ethanol (1.0 mmol) in dried acetonitril (10 mL) at under nitrogen is added triethylamine (1.5 mL), tri-o-tolylphosphine (0.4 mmol), styrene (2.0 mmol), and tri(debenzylideneacetone)dipalladiumn (0) (0.1 mmol). The reaction mixture is heated at 90° C. (oil bath) overnight. It is quenched with water and extracted with ethyl acetate. Extracts are dried over magnesium sulfate and concentrated under reduced pressure. The chromatography (silica gel) gives the title compound.

### COMPOUND 21: 2-(1-ETHYL-6-ISOPROPYL-8-PHENYLETHYNYL-1,3,4,9-TETRAHYDRO-PYRANO[3,4-B]INDOL-1-YL)-ETHANOL

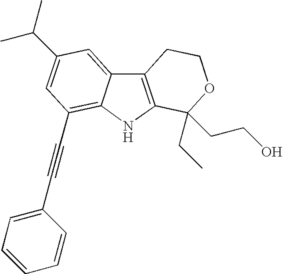

The title compound is prepared in a manner analogous to Example 20.A, except using phenylacetylene.

### COMPOUND 22: (1-ETHYL-1,3,4,9-TETRAHYDRO-PYRANO[3,4-B]INDOL-1-YL)-ETHANOL

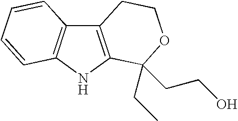

The title compound is prepared in a manner analogous to the procedure outlined below:

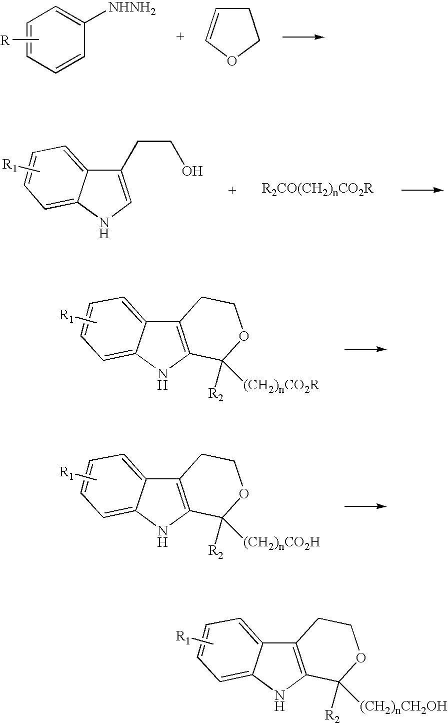

### 22.A. Synthesis of (1-Ethyl-1,3,4,9-tetrahydro-pyrano[3,4-b]indol-1-yl)-acetic acid ethyl ester

A mixture of tryptophol (1.612 g, 10 mmol), ethyl propionylacetate (1.730 g, 12 mmol), and p-toluenesulfonic acid monohydrate (0.20 g) in benzene (70 mL) was heated to reflux for 5 hours. It was quenched with ethyl acetate and washed with saturated sodium bicarbonate. The organic layer was dried over magnesium sulfate, evaporated to dryness. Flash chromatography on silica gel provided 1.943 g (68%) of the title compound as a solid. mp<80° C. ¹H NMR (300 MHz, CDCl₃) δ 9.06 (br, 1H), 7.50 (d, 1H), 7.36 (d, 1H), 7.14 (t, 1H), 7.12 (t, 1H), 4.18 (q, 2H), 4.03 (m, 1H), 3.94 (m, 1H), 2.99 (d, 1H), 2.88 (d, 1H), 2.78 (m, 2H), 2.14 (m, 1H), 2.01 (m, 1H), 1.25 (t, 3H), 0.82 (t, 3H); ESI (+) MS m/e=288 (MH⁺), ESI (−) MS m/e=286 (MH⁻).

### 22.B. Synthesis of (1-Ethyl-1,3,4,9-tetrahydro-pyrano[3,4-b]indol-1-yl)-acetic acid

To a solution of (1-ethyl-1,3,4,9-tetrahydro-pyrano[3,4-b]indol-1-yl)-acetic acid ethyl ester (2.50 g, 8.7 mmol) in 1,4-dioxane was added a solution of lithium hydroxide monohydrate (1.50 g, 35.7 mmol) in water (5 mL). The mixture was stirred at room temperature overnight. It was neutralized with 5% HCl solution and extracted with ethyl acetate. The extracts were washed with brine, dried over magnesium sulfate, and evaporated to dryness. Flash chromatography on silica gel provided 0.954 g (42%) of the title compound as a solid. mp. 135-136° C. ¹H NMR (500 MHz, CDCl₃) δ 10.0 (br, 1H), 8.55 (br, 1H), 7.51 (d, 1H), 7.34 (d, 1H), 7.18 (t, 1H), 7.12 (t, 1H), 4.12 (m, 1H), 4.06 (m, 1H), 3.01 (d, 1H), 2.99 (d, 1H), 2.85 (m, 2H), 2.10 (m, 1H), 2.03 (m, 1H), 0.86 (t, 3H); ESI (+) MS m/e=260 (MH⁺), ESI (−) MS m/e=258 (MH⁻).

### 22.C. Synthesis of (1-Ethyl-1,3,4,9-tetrahydro-pyrano[3,4-b]indol-1-yl)-ethanol

To solution of (1-ethyl-1,3,4,9-tetrahydro-pyrano[3,4-b]indol-1-yl)-acetic acid (0.52 g, 2.0 mmol) in tetrahydrofuran (10 mL) was added lithium aluminum hydride (0.114 g, 3.0 mmol) in several small portions. The mixture was stirred at room temperature for 6 hours. It was quenched with ethyl acetate carefully and washed with water. The organic layer was dried over magnesium sulfate and evaporated to dryness. Flash chromatography on silica gel provided 0.389 g (79%) of the title compound as an oil. ¹H NMR (500 MHz, CDCl₃) δ 7.82 (br, 1H), 7.52 (d, 1H), 7.34 (d, 1H), 7.18 (td, 1H), 7.13 (td, 1H), 4.07 (m, 1H), 4.01 (m, 1H), 3.70 (m, 1H), 3.64 (m, 1H), 2.89 (m, 1H), 2.77 (dt, 1H), 2.71 (br, 1H), 2.20 (m, 1H), 2.05 (m, 1H), 2.00 (m, 1H), 1.90 (m, 1H), 0.94 (t, 3H); ESI (+) MS m/e=246 (MH⁺), ESI (−) MS m/e=244 (MH⁻).

### COMPOUND 23: 2-(1-ETHYL-6-METHOXY-1,3,4,9-TETRAHYDRO-PYPANO[3,4-B]INDOL-1-YL)-ETHANOL

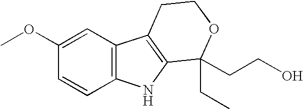

### 23.A. Synthesis of (1-Ethyl-6-methoxy-1,3,4,9-tetrahydro-pyrano[3,4-b]indol-1-yl)-acetic acid ethyl ester

The title compound was synthesized in a manner analogous to step 22, using 5-methoxytrypotophol as the 3-indolethanol component in step 22.A. ¹H NMR (300 MHz, CDCl₃) δ 8.93 (br, 1H), 7.25 (d, 1H), 6.95 (d, 1H), 6.90 (dd, 1H), 4.17 (q, 2H), 4.03 (m, 1H), 3.94 (m, 1H), 3.86 (s, 3H), 2.99 (d, 1H), 2.90 (d, 1H), 2.74 (m, 2H), 2.12 (m, 1H), 2.00 (m, 1H), 1.27 (t, 3H), 0.82 (t, 3H); ESI (+) MS m/e=318 (MH⁺), ESI (−) MS m/e=316 (MH⁻).

### 23.B. Synthesis of (1-Ethyl-6-methoxy-1,3,4,9-tetrahydro-pyrano[3,4-b]indol-1-yl)-acetic acid

The title compound was synthesized in a manner analogous to step 22, using (1-ethyl-6-methoxy-1,3,4,9-tetrahydro-pyrano[3,4-b]indol-1-yl)-acetic acid ethyl ester as the ester component in step 22.B., afforded the title compound as a solid. mp. 169° C. ¹H NMR (300 MHz, CDCl₃) δ 8.38 (br, 1H), 7.22 (d, 1H), 6.94 (d, 1H), 6.84 (dd, 1H), 4.08 (m, 2H), 3.85 (s, 3H), 2.97 (m, 2H), 2.81 (m, 2H), 2.02 (m, 2H), 0.85 (t, 3H); ESI (+) MS m/e=290 (MH⁺), ESI (−) MS m/e=288 (MH⁻).

### 23.C. Synthesis of 2-(1-Ethyl-6-methoxy-1,3,4,9-tetrahydro-pyrano[3,4-b]indol-1-yl)-ethanol

The title compound was synthesized in a manner analogous to step 22, using (1-ethyl-6-methoxy-1,3,4,9-tetrahydro-pyrano[3,4-b]indol-1-yl)-acetic acid as the carboxylic acid component in step 22.C., afforded the title compound as a solid. ¹H NMR (500 MHz, CDCl₃) δ 7.71 (br, 1H), 7.22 (d, 1H), 6.97 (d, 1H), 6.83 (dd, 1H), 4.08 (m, 1H), 4.00 (m, 1H), 3.86 (s, 3H), 3.69 (m, 1H), 3.64 (m, 1H), 2.85 (m, 1H), 2.73 (dt, 1H), 2.19 (m, 1H), 2.05 (br, 1H), 2.03 (m, 1H), 1.98 (m, 1H), 1.89 (m, 1H), 0.93 (t, 3H); ESI (+) MS m/e=276 (MH⁺), ESI (−) MS m/e=274 (MH⁻).

### COMPOUND 24: 2-(1-ETHYL-6-METHYL-1,3,4,9-TETRAHYDRO-PYPANO[3,4-B]INDOL-1-YL)-ETHANOL

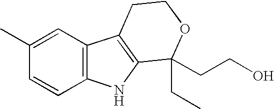

### 24.A. Synthesis of 2-(5-Methyl-1H-indol-3-yl)-ethanol

To a suspension of 4-methylphenylhydrazine hydrochloride (2.50 g, 15.7 mmol) in 1,4-dioxane (25 mL) and water (1.5 mL) was dropped neat 2,3-dihydrofuran (1.66 g, 23.6 mmol). After the addition, the mixture was heated at 95° C. for 4 hours. After cooling to room temperature, it was poured into ethyl ether, dried over magnesium sulfate, evaporated to dryness. Flash chromatography on silica gel provided 0.485 g (18%) of the title compound as a solid. ¹H NMR (500 MHz, CDCl₃) δ 7.95 (br, 1H), 7.41 (s, 1H), 7.27 (d, 1H), 7.05 (m, 2H), 3.90 (dd, 2H), 3.01 (t, 2H), 2.46 (s, 3H), 1.50 (t, br, 1H).

### 24.B. Synthesis of (1-Ethyl-6-methyl-1,3,4,9-tetrahydro-pyrano[3,4-b]indol-1-yl)-acetic acid ethyl ester

To a solution of 2-(5-methyl-1H-indol-3-yl)-ethanol (0.48 g, 2.7 mmol) in dichloromethane (10 mL) was added boron trifluoride diethyl etherate (0.468 g, 3.3 mmol), followed by ethyl propionylacetate (0.649 g, 4.5 mmol). The mixture was stirred at room temperature for 5 hours. It was quenched with saturated sodium bicarbonate solution and extracted with dichloromethane. The organic layer was dried over magnesium sulfate and evaporated to dryness. Flash chromatography on silica gel provided 0.421 g (52%) of the title compound as an oil. ¹H NMR (500 MHz, CDCl₃) δ 8.90 (br, 1H), 7.28 (s, 1H), 7.24 (d, 1H), 6.99 (d, 1H), 4.16 (m, 2H), 4.03 (m, 1H), 3.94 (m, 1H), 2.98 (d, 1H), 2.88 (d, 1H), 2.80 (m, 1H), 2.73 (m, 1H), 2.44 (s, 3H), 2.12 (m, 1H), 1.98 (m, 1H), 1.25 (t, 3H), 0.80 (t, 3H); ESI (−) MS m/e=300 (MH⁻).

### 24.C. Synthesis of (1-Ethyl-6-methyl-1,3,4,9-tetrahydro-pyrano[3,4-b]indol-1-yl)-acetic acid

The title compound was synthesized in a manner analogous to step 22.B., using (1-ethyl-6-methyl-1,3,4,9-tetrahydro-pyrano[3,4-b]indol-1-yl)-acetic acid ethyl ester as the ester component afforded the title compound as a solid. mp. 158-159° C. ¹H NMR (500 MHz, CDCl₃) δ 9.70 (br, 1H), 8.33 (br, 1H), 7.29 (s, 1H), 7.22 (d, 1H), 7.00 (d, 1H), 4.10 (m, 1H), 4.05 (m, 1H), 2.99 (d, 1H), 2.98 (d, 1H), 2.81 (q, 2H), 2.44 (s, 3H), 2.07 (m, 1H), 2.01 (m, 1H), 0.85 (t, 3H); ESI (+) MS m/e=274 (MH⁺), ESI (−) MS m/e=272 (MH⁻).

### 24.D. Synthesis of 2-(1-Ethyl-6-methyl-1,3,4,9-tetrahydro-pyrano[3,4-b]indol-1-yl)-ethanol

The title compound was synthesized in a manner analogous to step 22.C., using (1-ethyl-6-methyl-1,3,4,9-tetrahydro-pyrano[3,4-b]indol-1-yl)-acetic acid as the carboxylic acid component afforded the title compound as a solid. mp. 114-115° C. ¹H NMR (500 MHz, CDCl₃) δ 7.70 (br, 1H), 7.30 (s, 1H), 7.21 (d, 1H), 7.00 (dd, 1H), 4.06 (m, 1H), 3.97 (m, 1H), 3.67 (m, 1H), 3.62 (m, 1H), 2.84 (m, 1H), 2.73 (m, 1H), 2.71 (br, 1H), 2.45 (s, 3H), 2.17 (m, 1H), 2.04 (m, 1H), 1.96 (m, 1H), 1.86 (m, 1H), 0.92 (t, 3H); ESI (+) MS m/e=260 (MH⁺), ESI (−) MS m/e=258 (MH⁻).

### COMPOUND 25: 2-(1-ETHYL-8-METHYL-1,3,4,9-TETRAHYDRO-PYRANO[3,4-B]INDOL-1-YL)-ETHANOL

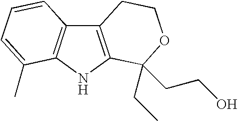

### 25.A. Synthesis of 2-(7-Methyl-1H-indol-3-yl)-ethanol

Following the procedure of example 24.A. except using 2-methylphenylhydrazine hydrochloride as the hydrazine component afforded the title compound as a solid. ¹H NMR (500 MHz, CDCl₃) δ 7.97 (br, 1H), 7.49 (d, 1H), 7.11 (d, 1H), 7.07 (t, 1H), 7.03 (d, 1H), 3.91 (t, 2H and br, 1H), 3.04 (t, 2H), 2.49 (s, 3H).

### 25.B. Synthesis of (1-Ethyl-8-methyl-1,3,4,9-tetrahydro-pyrano[3,4-b]indol-1-yl)-acetic acid ethyl ester

Following the procedure of example 23.B. except using 2-(7-methyl-1H-indol-3-yl)-ethanol as the 3-indolethanol component afforded the title compound as solid. mp. 77-78° C. ¹H NMR (500 MHz, CDCl₃) δ 9.04 (br, 1H), 7.36 (d, 1H), 7.02 (t, 1H), 6.97 (d, 1H), 4.19 (m, 2H), 4.04 (m, 1H), 3.94 (m, 1H), 2.98 (d, 1H), 2.90 (d, 1H), 2.81 (m, 1H), 2.75 (dt, 1H), 2.49 (s, 3H), 2.15 (m, 1H), 2.02 (m, 1H), 1.27 (t, 3H), 0.83 (t, 3H); ESI (−) MS m/e=300 (MH⁻).

### 25.C. Synthesis of 2-(1-Ethyl-8-methyl-1,3,4,9-tetrahydro-pyrano[3,4-b]indol-1-yl)-ethanol.

Following the procedure of example 22.C. except using (1-ethyl-8-methyl-1,3,4,9-tetrahydro-pyrano[3,4-b]indol-1-yl)-acetic acid ethyl ester as the carboxylic acid component afforded the title compound as a solid. mp. 68° C. ¹H NMR (500 MHz, CDCl₃) δ 7.68 (br, 1H), 7.37 (d, 1H), 7.05 (t, 1H), 6.98 (d, 1H), 4.06 (m, 1H), 3.98 (m, 1H), 3.70 (m, 1H), 3.65 (m, 1H), 2.88 (m, 1H), 2.76 (t, 1H), 2.72 (m, 1H), 2.47 (s, 3H), 2.21 (m, 1H), 2.07 (m, 1H), 2.00 (m, 1H), 1.91 (m, 1H), 0.94 (t, 3H); ESI (+) MS m/e=260 (MH⁺), ESI (−) MS m/e=258 (MH⁻).

### COMPOUND 26: 2-(1-ETHYL-8-FLUORO-1,3,4,9-TETRAHYDRO-PYRANO[3,4-B]INDOL-1-YL)-ETHANOL

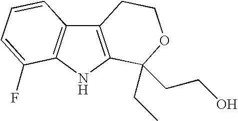

### 26.A. Synthesis of 2-(7-Fluoro-1H-indol-3-yl)-ethanol

Following the procedure of example 24.A. except using 2-fluorophenylhydrazine hydrochloride as the hydrazine component afforded the title compound as an oil.

### 26.B. Synthesis of (1-Ethyl-8-fluoro-1,3,4,9-tetrahydro-pyrano[3,4-b]indol-1-yl)-acetic acid ethyl ester

Following the procedure of 24.B. except using 2-(7-fluoro-1H-indol-3-yl)-ethanol as the 3-indolethanol component afforded the title compound as an oil.

### 26.C. Synthesis of 2-(1-Ethyl-8-fluoro-1,3,4,9-tetrahydro-pyrano[3,4-b]indol-1-yl)-ethanol

Following the procedure of example 22.C. except using (1-ethyl-8-fluoro-1,3,4,9-tetrahydro-pyrano[3,4-b]indol-1-yl)-acetic acid ethyl ester as the carboxylic acid component afforded the title compound as a solid. ¹H NMR (500 MHz, CDCl₃) δ 8.18 (br, 1H), 7.27 (d, 1H), 7.02 (m, 1H), 6.89 (dd, 1H), 4.07 (m, 1H), 3.99 (m, 1H), 3.71 (m, 1H), 3.65 (m, 1H), 2.88 (m, 1H), 2.78 (dt, 1H), 2.76 (br, 1H), 2.22 (m, 1H), 2.07 (m, 1H), 1.99 (m, 1H), 1.91 (m, 1H), 0.94 (t, 3H); ESI (+) MS m/e=264 (MH⁺), ESI (−) MS m/e=262 (MH⁻).

### COMPOUND 27: 2-(8-CHLORO-1-ETHYL-1,3,4,9-TETRAHYDRO-PYRANO[3,4-B]INDOL-1-YL)-ETHANOL

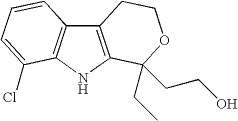

### 27.A. Synthesis of 2-(7-Chloro-1H-indol-3-yl)-ethanol

Following the procedure of 24.A. except using 2-chlorophenylhydrazine hydrochloride as the hydrazine component afforded the title compound as an oil. ¹H NMR (500 MHz, CDCl₃) δ 8.26 (br, 1H), 7.52 (d, 1H), 7.21 (d, 1H), 7.15 (d, 1H), 7.06 (t, 1H), 3.91 (t, 2H), 3.02 (t, 2H), 1.48 (br, 1H).

### 27.B. Synthesis of (8-Chloro-1-ethyl-1,3,4,9-tetrahydro-pyrano[3,4-b]indol-1-yl)-acetic acid ethyl ester

Following the procedure of 24.B. except using 2-(7-chloro-1H-indol-3-yl)-ethanol as the 3-indolethanol component afforded the title compound as a solid. ¹H NMR (500 MHz, CDCl₃) δ 9.28 (br, 1H), 7.39 (d, 1H), 7.16 (d, 1H), 7.02 (t, 1H), 4.18 (m, 2H), 4.05 (m, 1H), 3.94 (m, 1H), 2.98 (d, 1H), 2.88 (d, 1H), 2.82 (m, 1H), 2.75 (dt, 1H), 2.15 (m, 1H), 2.03 (m, 1H), 1.27 (t, 3H), 0.84 (t, 3H); ESI (−) MS m/e=230 (MH⁻).

### 27.C. Synthesis of (8-Chloro-1-ethyl-1,3,4,9-tetrahydro-pyrano[3,4-b]indol-1-yl)-acetic acid

Following the procedure of example 22.B. except using (8-chloro-1-ethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 8.81 (br, 1H), 7.40 (d, 1H), 7.17 (d, 1H), 7.04 (t, 1H), 4.09 (m, 1H), 4.03 (m, 1H), 3.05 (d, 1H), 3.02 (d, 1H), 2.82 (m, 2H), 2.13 (m, 1H), 2.06 (m, 1H), 0.88 (t, 3H); ESI (+) MS m/e=294 (MH⁺), ESI (−) MS m/e=292 (MH⁻).

### 27.D. Synthesis of 2-(8-Chloro-1-ethyl-1,3,4,9-tetrahydro-pyrano[3,4-b]indol-1-yl)-ethanol

Following the procedure of example 22.C. except using (8-chloro-1-ethyl-1,3,4,9-tetrahydro-pyrano[3,4-b]indol-1-yl)-acetic acid as the carboxylic acid component afforded the title compound as a solid. ¹H NMR (500 MHz, CDCl₃) δ 8.05 (br, 1H), 7.41 (d, 1H), 7.17 (d, 1H), 7.05 (t, 1H), 4.07 (m, 1H), 4.00 (m, 1H), 3.72 (m, 1H), 3.67 (m, 1H), 2.87 (m, 1H), 2.76 (dt, 1H), 2.70 (br, 1H), 2.23 (m, 1H), 2.03 (m, 1H), 1.91 (m, 1H), 0.94 (t, 3H); ESI (+) MS m/e=280 (MH⁺), ESI (−) MS m/e=278 (MH⁻).

### COMPOUND 28: 2-(8-BROMO-1-ETHYL-1,3,4,9-TETRAHYDRO-PYRANO[3,4-B]INDOL-1-YL)-ETHANOL

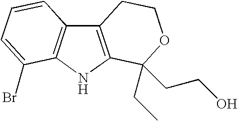

### 28.A. Synthesis of 2-(7-Bromo-1H-indol-3-yl)-ethanol

Following the procedure of example 24.A. except using 2-bromophenylhydrazine hydrochloride as the hydrazine component afforded the title compound as an oil.

### 28.B. Synthesis of (8-Bromo-1-ethyl-1,3,4,9-tetrahydro-pyrano[3,4-b]indol-1-yl)-acetic acid ethyl ester

Following the procedure of 24.B. except using 2-(7-bromo-1H-indol-3-yl)-ethanol as the 3-indolethanol component afforded the title compound as an oil.

### 28.C. Synthesis of 2-(8-Bromo-1-ethyl-1,3,4,9-tetrahydro-pyrano[3,4-b]indol-1-yl)-ethanol

To a solution of (8-bromo-1-ethyl-1,3,4,9-tetrahydro-pyrano[3,4-b]indol-1-yl)-acetic acid ethyl ester (1.03 g, 2.8 mmol) in tetrahydrofuran at room temperature was added 2.0 M solution of lithium borohydride in tetrahydrofuran. The mixture was heated to reflux for 5 hours. It was quenched with 5% HCl solution, followed by saturated sodium bicarbonate. It was extracted with ethyl acetate, extracts were dried over magnesium sulfate, and it was evaporated to dryness. Crystallization with diethyl ether afforded 0.682 g (75%) of the title compound as a solid. ¹H NMR (500 MHz, CDCl₃) δ 8.01 (br, 1H), 7.45 (d, 1H), 7.32 (d, 1H), 7.00 (t, 1H), 4.07 (m, 1H), 3.99 (m, 1H), 3.72 (m, 1H), 3.67 (m, 1H), 2.87 (m, 1H), 2.75 (dt, 1H), 2.68 (dd, 1H), 2.24 (m, 1H), 2.08 (m, 1H), 2.02 (m, 1H), 1.93 (m, 1H), 0.94 (t, 3H); ESI (+) MS m/e=324 (MH⁺), ESI (−) MS m/e=322 (MH⁻).

### COMPOUND 29: 2-(8-ETHYL-1-METHYL-1,3,4,9-TETRAHYDRO-PYRANO[3,4-B]INDOL-1-YL)-ETHANOL

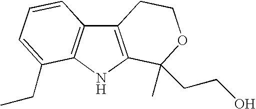

### 29.A. Synthesis of 2-(7-Ethyl-1H-indol-3-yl)-ethanol

Following the procedure of example 24.A. except using 2-ethylphenylhydrazine hydrochloride as the hydrazine component afforded the title compound as a solid. ¹H NMR (500 MHz, CDCl₃) δ 8.05 (br, 1H), 7.50 (d, 1H), 7.08 (m, 3H), 3.92 (m, 2H), 3.04 (m, 2H), 2.86 (m, 2H), 2.06 (br, 1H), 1.35 (t, 3H); ESI (+) MS m/e=190 (MH⁺), ESI (−) MS m/e=188 (MH⁻).

### 29.B. Synthesis of (8-Ethyl-1-methyl-1,3,4,9-tetrahydro-pyrano[3,4-b]indol-1-yl)-acetic acid ethyl ester

Following the procedure of example 22.A. except using 2-(7-ethyl-1H-indol-3-yl)-ethanol as the 3-indolethanol component and the ethyl acetoacetate as ketone component afforded the title compound as an oil. ¹H NMR (500 MHz, CDCl₃) δ 9.16 (br, 1H), 7.35 (d, 1H), 7.01 (m, 2H), 4.17 (m, 2H), 4.02 (m, 2H), 2.85 (m, 6H), 1.57 (t, 3H), 1.36 (t, 3H), 1.29 (t, 3H); ESI (+) MS m/e=302 (MH⁺), ESI (−) MS m/e=300 (MH⁻).

### 29.C. Synthesis of (8-Ethyl-1-methyl-1,3,4,9-tetrahydro-pyrano[3,4-b]indol-1-yl)-acetic acid

Following the procedure of example 1, step (b) except using (8-ethyl-1-methyl-1,3,4,9-tetrahydro-pyrano[3,4-b]indol-1-yl)-acetic acid ethyl ester as the ester component afforded the title compound as a solid. ESI (−) MS m/e=272 (MH⁻).

### 29.D. Synthesis of 2-(8-Ethyl-1-methyl-1,3,4,9-tetrahydro-pyrano[3,4-b]indol-1-yl)-ethanol

Following the procedure of example 22.C. except using (8-ethyl-1-methyl-1,3,4,9-tetrahydro-pyrano[3,4-b]indol-1-yl)-acetic acid as the carboxylic acid component afforded the title compound as a solid. ¹H NMR (500 MHz, CDCl₃) δ 7.74 (br, 1H), 7.37 (d, 1H), 7.09 (m, 1H), 7.03 (d, 1H), 4.12 (m, 1H), 3.98 (m, 1H), 3.70 (m, 2H), 2.92 (m, 1H), 2.85 (m, 2H), 2.74 (m, 2H), 2.15 (m, 2H), 1.56 (s, 3H), 1.36 (t, 3H); ESI (+) MS m/e=282 (MNa⁺), ESI (−) MS m/e=258 (MH⁻).

### COMPOUND 30: 2-(8-ETHYL-1-PROPYL-1,3,4,9-TETRAHYDRO-PYRANO[3,4-B]INDOL-1-YL)-ETHANOL

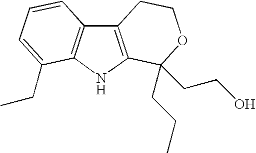

### 30.A. Synthesis of (8-Ethyl-1-propyl-1,3,4,9-tetrahydro-pyrano[3,4-b]indol-1-yl)-acetic acid ethyl ester

Following the procedure of example 1, step (a) except using 2-(7-ethyl-1H-indol-3-yl)-ethanol as the 3-indolethanol component and the ethyl butyrylacetate as ketone component afforded the title compound as a solid. ¹H NMR (500 MHz, CDCl₃) δ 9.10 (br, 1H), 7.34 (d, 1H), 7.03 (m, 2H), 4.17 (m, 2H), 4.02 (m, 1H), 3.92 (m, 1H), 2.99 (d, 1H), 2.84 (m, 3H), 2.73 (dt, 1H), 2.09 (m, 1H), 1.96 (m, 1H), 1.35 (t, 3H), 1.26 (t, 3H), 1.19 (m, 2H), 0.85 (t, 3H); ESI (+) MS m/e=330 (MH⁺), ESI (−) MS m/e=328 (MH⁻).

### 30.B. Synthesis of (8-Ethyl-1-propyl-1,3,4,9-tetrahydro-pyrano[3,4-b]indol-1-yl)-acetic acid

Following the procedure of example 22.B. except using (8-ethyl-1-propyl-1,3,4,9-tetrahydro-pyrano[3,4-b]indol-1-yl)-acetic acid ethyl ester as the ester component afforded the title compound as a solid.

### 30.C. Synthesis of 2-(8-Ethyl-1-propyl-1,3,4,9-tetrahydro-pyrano[3,4-b]indol-1-yl)-ethanol

Following the procedure of example 22.C. except using (8-ethyl-1-propyl-1,3,4,9-tetrahydro-pyrano[3,4-b]indol-1-yl)-acetic acid as the carboxylic acid component afforded the title compound as a solid. ¹H NMR (500 MHz, CDCl₃) δ 7.73 (br, 1H), 7.36 (d, 1H), 7.09 (t, 1H), 7.02 (m, 1H), 4.05 (m, 1H), 4.01 (m, 1H), 3.72 (m, 1H), 3.67 (m, 1H), 2.85 (m, 2H), 2.76 (dt, 1H), 2.68 (br, 1H), 2.20 (m, 1H), 2.09 (m, 1H), 1.90 (m, 2H), 1.48 (m, 1H), 1.36 (t, 3H), 1.32 (m, 1H), 0.91 (t, 3H); ESI (+) MS m/e=288 (MH⁺), ESI (−) MS m/e=286 (MH⁻).

### COMPOUND 31: 2-(8-ETHYL-1-ISOPROPYL-1,3,4,9-TETRAHYDRO-PYRANO[3,4-B]INDOL-1-YL)-ETHANOL

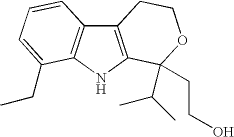

### 31.A. Synthesis of (8-Ethyl-1-isopropyl-1,3,4,9-tetrahydro-pyrano[3,4-b]indol-1-yl)-acetic acid ethyl ester

Following the procedure of example 22.A. except using 2-(7-ethyl-1H-indol-3-yl)-ethanol as the 3-indolethanol component and ethyl iso-butyrylacetate as ketone component afforded the title compound as a solid. ¹H NMR (500 MHz, CDCl₃) δ 9.12 (br, 1H), 7.36 (d, 1H), 7.07 (m, 2H), 4.13 (m, 3H), 3.81 (m, 1H), 3.04 (q, 2H), 2.87 (m, 3H), 2.66 (m, 1H), 2.56 (m, 1H), 1.37 (t, 3H), 1.25 (t, 3H), 1.05 (d, 3H), 0.69 (d, 3H).

### 31.B. Synthesis of (8-Ethyl-1-isopropyl-1,3,4,9-tetrahydro-pyrano[3,4-b]indol-1-yl)-acetic acid

Following the procedure of example 24.B. except using (8-ethyl-1-isopropyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 9.70 (br, 1H), 8.55 (br, 1H), 7.36 (d, 1H), 7.07 (dd, 1H), 7.01 (d, 1H), 4.18 (m, 1H), 3.94 (m, 1H), 3.10 (q, 2H), 2.82 (m, 4H), 2.52 (m, 1H), 1.32 (t, 3H), 1.06 (d, 3H), 0.82 (d, 3H); ESI (+) MS m/e=302 (MH⁺), ESI (−) MS m/e=300 (MH⁻).

### 31.C. Synthesis of 2-(8-Ethyl-1-isopropyl-1,3,4,9-tetrahydro-pyrano[3,4-b]indol-1-yl)-ethanol

Following the procedure of example 22.C. except using (8-ethyl-1-isopropyl-1,3,4,9-tetrahydro-pyrano[3,4-b]indol-1-yl)-acetic acid as the carboxylic acid component afforded the title compound as a solid. ¹H NMR (500 MHz, CDCl₃) δ 7.68 (br, 1H), 7.38 (d, 1H), 7.09 (dd, 1H), 7.03 (d, 1H), 4.06 (m, 2H), 3.65 (m, 2H), 2.87 (m, 3H), 2.77 (dt, 1H), 2.68 (br, 1H), 2.32 (m, 1H), 2.23 (m, 1H), 2.05 (m, 1H), 1.35 (t, 3H), 1.05 (d, 3H), 1.00 (d, 3H); ESI (+) MS m/e=288 (MH⁺), ESI (−) MS m/e=286 (MH⁻).

### COMPOUND 32: 2-(8-Ethyl-1-phenyl-1,3,4,9-tetrahydro-pyrano[3,4-b]indol-1-yl)-ethanol

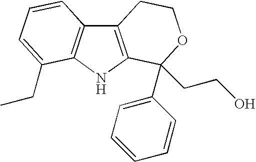

### 32.A. Synthesis of (8-Ethyl-1-phenyl-1,3,4,9-tetrahydro-pyrano[3,4-b]indol-1-yl)-acetic acid ethyl ester

Following the procedure of example 22.A. except using 2-(7-ethyl-1H-indol-3-yl)-ethanol as the 3-indolethanol component and ethyl benzoylacetate as ketone component afforded the title compound as a solid. ¹H NMR (500 MHz, CDCl₃) δ 10.05 (br, 1H), 7.42 (d, 1H), 7.28 (m, 5H), 7.12 (m, 2H), 3.96 (m, 3H), 3.60 (m, 1H), 3.43 (d, 1H), 3.22 (d, 1H), 3.05 (m, 3H), 2.65 (dd, 1H), 1.42 (d, 3H), 1.03 (t, 3H); ESI (−) MS m/e=362 (MH⁻).

### 32.B. Synthesis of (8-Ethyl-1-phenyl-1,3,4,9-tetrahydro-pyrano[3,4-b]indol-1-yl)-acetic acid

Following the procedure of example 22.B. except using (8-ethyl-1-phenyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 9.25 (br, 1H), 7.41 (d, 1H), 7.31 (m, 5H), 7.10 (t, 1H), 7.06 (d, 1H), 4.08 (m, 1H), 3.70 (m, 1H), 3.42 (d, 1H), 3.22 (d, 1H), 3.03 (m, 1H), 2.83 (m, 2H), 2.68(m, 1H), 1.33 (t, 3H); ESI (+) MS m/e=358 (MNa⁺). ESI (−) MS m/e=334 (MH⁻).

### 32.C. Synthesis of 2-(8-Ethyl-1-phenyl-1,3,4,9-tetrahydro-pyrano[3,4-b]indol-1-yl)-ethanol

Following the procedure of example 22.C. except using (8-ethyl-1-phenyl-1,3,4,9-tetrahydro-pyrano[3,4-b]indol-1-yl)-acetic acid as the carboxylic acid component afforded the title compound as a solid. ¹H NMR (500 MHz, CDCl₃) δ 8.27 (br, 1H), 7.38 (m, 3H), 7.32 (m, 3H), 7.12 (t, 1H), 7.08 (d, 1H), 4.12 (m, 2H), 3.99 (dd, 1H), 3.61 (ddd, 1H), 3.01 (m, 1H), 2.94 (q, 2H), 2.61 (m, 2H), 2.47(m, 1H), 1.39 (t, 3H).

### COMPOUND 34: [8′-ETHYL-4′,9′-DIHYDRO-3′H-SPIRO(CYCLOHEXANE-1,1′-PYRANO[3,4-B]INDOL)-4-YL]-METHANOL

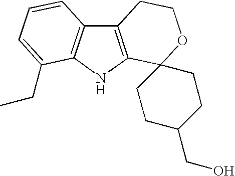

### 34.A. Synthesis of 8′-Ethyl-4′,9′-dihydro-3′H-spiro(cyclohexane-1,1′-pyrano[3,4-b]indole)-4-carboxylic acid ethyl ester

Following the procedure of example 24.A. except using 2-(7-ethyl-1H-indol-3-yl)-ethanol as the 3-indolethanol component and 4-oxo-cyclohexane carboxylic acid ethyl ester as ketone component afforded the title compound as an oil. ESI (+) MS m/e=342 (MH⁺), ESI (−) MS m/e=340 (MH⁻).

### 34.B. Synthesis of 8′-Ethyl-4,9′-dihydro-3′H-spiro(cyclohexane-1,1′-pyrano[3,4-b]indole)-4-carboxylic acid

Following the procedure of example 22.B. except using 8′-ethyl-4′,9′-dihydro-3′H-spiro(cyclohexane-1,1′-pyrano[3,4-b]indole)-4-carboxylic acid ethyl ester as the ester component afforded the title compound as a solid. ESI (+) MS m/e=314 (MH⁺), ESI (−) MS m/e=312 (MH⁻).

### 34.C. Synthesis of [8′-Ethyl-4′,9′-dihydro-3′H-spiro(cyclohexane-1,1′-pyrano[3,4-b]indol)-4-yl]-methanol

Following the procedure of example 22.C. except using 8′-ethyl-4′,9′-dihydro-3′H-spiro(cyclohexane-1,1′-pyrano[3,4-b]indole)-4-carboxylic acid as the carboxylic acid component afforded the title compound as a solid. ¹H NMR (500 MHz, CDCl₃) δ 7.51 (br, 1H), 7.33 (d, 1H), 7.06 (t, 1H), 7.00 (d, 1H), 3.97 (t, 2H), 3.54 (t, 2H), 2.84 (q, 2H), 2.78 (t, 2H), 2.12 (br, 1H), 2.09 (m, t, 1H), 1.69 (m, 4H), 1.60 (m, 1H), 1.52 (m, 3H), 1.35 (t, 3H); ESI (+) MS m/e=300 (MH⁺), ESI (−) MS m/e=298 (MH⁻).

### COMPOUND 35: R-2-(1,8-DIETHYL-1,3,4,9-TETRAHYDRO-PYRANO[3,4-B]INDOL-1-YL)-ETHANOL

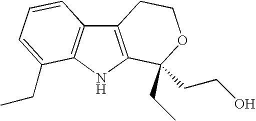

### 35.A. Synthesis of R-2-(1,8-Diethyl-1,3,4,9-tetrahydro-pyrano[3,4-b]indol-1-yl)-ethanol

Following the procedure of example 22, except using R-(1,8-Diethyl-1,3,4,9-tetrahydro-pyrano[3,4-b]indol-1-yl)-acetic acid as the carboxylic acid component in step 22.C. afforded the title compound as a solid. ¹H NMR (500 MHz, CDCl₃) δ 7.74 (br, 1H), 7.37 (d, 1H), 7.09 (t, 1H), 7.03 (d, 1H), 4.07 (m, 1H), 3.98 (m, 1H), 3.68 (m, 2H), 2.86 (m, 3H), 2.76 (dt, 1H), 2.69 (br, t, 1H), 2.21 (m, 1H), 2.07 (m, 1H), 2.00 (m, 1H), 1.91 (m, 1H), 1.35 (t, 3H), 0.94 (t, 3H); ESI (+) MS m/e=274 (MH⁺), ESI (−) MS m/e=272 (MH⁻).

### COMPOUND 36: 2-(1-ETHYL-8-ISOPROPYL-1,3,4,9-TETRAHYDRO-PYRANO[3,4-B]INDOL-1-YL)-ETHANOL

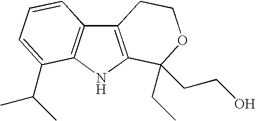

### 36.A. Synthesis 2-Hydroxyimino-N-(2-isopropyl-phenyl)-acetamide

A mixture of 2-isopropylaniline (4.7 g, 35 mmol), Na₂SO₄ (30.0 g), concentrated hydrochloride (3 mL), chloral hydrate (6.5 g), hydroxylamine hydrochloride (8.00 g) in water (150 mL) was heated at 85° C. for 40 minutes. After cooling to room temperature, it was extracted with ethyl acetate. The extracts were dried over magnesium sulfate and evaporated to dryness. Flash chromatography on silica gel provided 4.357 g (54%) of the title compound as solid.

### 36.B. Synthesis of 7-Isopropyl-1H-indole-2,3-dione

To concentrated sulfuric acid at 80° C. was added 2-hydroxyimino-N-(2-isopropyl-phenyl)-acetamide in several small portions over 10 minutes. After addition it was heated at 80° C. for 30 minutes., then poured into ice. Filtration, washing with water, and drying under vacuum over P₂O₅ provided 2.974 g (84%) of the title compound as a solid. ESI (−) MS m/e=188 (MH⁻).

### 36.C. Synthesis of (7-Isopropyl-1H-indol-3-yl)-oxo-acetic acid ethyl ester

To a solution of 7-isopropyl-1H-indole-2,3-dione (2.97 g, 15.7 mmol) in tetrahydrofuran (20 mL) was dropped 2.0 M solution of lithium borohydride in tetrahydrofuran (15 mL, 30 mmol). The mixture was heated at 90° C. for 4 hours. It was quenched with 5% HCl, followed by saturated sodium bicarbonate. It was extracted with ethyl acetate. The extracts were dried over magnesium sulfate and evaporated to dryness to provide a crude 7-isopropyl-1H-indole. To a solution of the crude 7-isopropyl-1H-indole in diethyl ether (40 mL) was dropped 2.0 M solution of oxalyl chloride in dichloromethane (15 mL, 30 mmol). After stirring at room temperature for 5 hours, it was evaporated to dryness. Ethanol was added to the residue and it was stirred at room temperature overnight. After the ethanol was evaporated, flash chromatography on silica gel provided 0.972 g (24%) of the title compound as solid. ESI (−) MS m/e=258 (MH⁻).

### 36.D. Synthesis of (1-Ethyl-8-isopropyl-1,3,4,9-tetrahydro-pyrano[3,4-b]indol-1-yl)-acetic acid ethyl ester

To a solution of (7-isopropyl-1H-indol-3-yl)-oxo-acetic acid ethyl ester (0.97 g. 3.7 mmol) in tetrahydrofuran was added 2.0 M solution of lithium borohydride in tetrahydrofuran. The mixture was heated at 90° C. for 5 hours. It was quenched with 5% HCl, followed by saturated sodium bicarbonate. It was extracted with ethyl acetate. The extracts were dried over magnesium sulfate and evaporated to dryness to provide a crude 2-(7-isopropyl-1H-indol-3-yl)-ethanol. ESI (+) MS m/e=204 (MH⁺), ESI (−) MS m/e=202 (MH⁻).

Following the procedure of example 24.B. except using 2-(7-isopropyl-1H-indol-3-yl)-ethanol as the 3-indolethanol component afforded the title compound as an oil. ESI (+) MS m/e=330 (MH⁺), ESI (−) MS m/e=328 (MH⁻).

### 36.E. Synthesis of (1-Ethyl-8-isopropyl-1,3,4,9-tetrahydro-pyrano[3,4-b]indol-1-yl)-acetic acid

Following the procedure of example 22.B. except using (1-ethyl-8-isopropyl-1,3,4,9-tetrahydro-pyrano[3,4-b]indol-1-yl)-acetic acid ethyl ester as the ester component afforded the title compound as a solid. mp. 158-159° C. ¹H NMR (500 MHz, CDCl₃) δ 9.50 (br, 1H), 8.58 (br, 1H), 7.36 (d, 1H), 7.08 (m, 2H), 4.09 (m, 1H), 4.04 (m, 1H), 3.20 (m, 1H), 3.05 (d, 1H), 3.02 (d, 1H), 2.84 (m, 2H), 2.13 (m, 1H), 2.04 (m, 1H), 1.38 (d, 3H), 1.35 (d, 3H), 0.88 (t, 3H); ESI (+) MS m/e=302 (MH⁺), ESI (−) MS m/e=300 (MH⁻).

### 36. F Synthesis of 2-(1-Ethyl-8-isopropyl-1,3,4,9-tetrahydro-pyrano[3,4-b]indol-1-yl)-ethanol

Following the procedure of example 22.C. except using (1-ethyl-8-isopropyl-1,3,4,9-tetrahydro-pyrano[3,4-b]indol-1-yl)-acetic acid as the carboxylic acid component afforded the title compound as a oil. ¹H NMR (500 MHz, CDCl₃) δ 7.78 (br, 1H), 7.36 (d, 1H), 7.11 (t, 1H), 7.07 (d, 1H), 4.07 (m, 1H), 3.99 (m, 1H), 3.71 (m, 2H), 3.20 (m, 1H), 2.90 (m, 1H), 2.76 (dt, 1H), 2.65 (br, 1H), 2.22 (m, 1H), 2.06 (m, 1H), 2.03 (m, 1H), 1.92 (m, 1H), 1.38 (d, 6H), 0.88 (t, 3H); ESI (+) MS m/e=288 (MH⁺), ESI (−) MS m/e=286 (MH⁻).

### COMPOUND 37: 2-(1-ETHYL-8-TRIFLUOROMETHYL-1,3,4,9-TETRAHYDRO-PYRANO[3,4-B]INDOL-1-YL)-ETHANOL

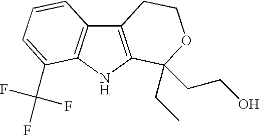

### 37.A. Synthesis of 2-Hydroxyimino-N-(2-trifluoromethyl-phenyl)-acetamide

Following the procedure of example 36.A. except using 2-trifluoromethylaniline as the aniline component afforded the title compound as a solid.

### 37.B. Synthesis of 7-Trifluoromethyl-1H-indole-2,3-dione

Following the procedure of example 36.B. except using 2-hydroxyimino-N-(2-trifluoromethyl-phenyl)-acetamide as the acetamide component afforded the title compound as a solid. ESI (−) MS m/e=214 (MH⁻).

### 37.C. Synthesis of (7-Trifluoromethyl-1H-indol-3-yl)-oxo-acetic acid ethyl ester

Following the procedure of example 36.C. except using 7-trifluoromethyl-1H-indole-2,3-dione as the dione component afforded the title compound as a solid.

### 37.D. Synthesis of 2-(1-Ethyl-8-trifluoromethyl-1,3,4,9-tetrahydro-pyrano[3,4-b]indol-1-yl)-ethanol

Following the procedure of example 28.C. except using (1-ethyl-8-trifluoromethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 8.42 (br, 1H), 7.67 (d, 1H), 7.41 (d, 1H), 7.18 (t, 1H), 4.07 (m, 1H), 4.00 (m, 1H), 3.71 (m, 2H), 2.89 (m, 1H), 2.78 (dt, 1H), 2.64 (br, 1H), 2.23 (m, 1H), 2.07 (m, 1H), 2.02 (m, 1H), 1.93 (m, 1H), 0.93 (t, 3H); ESI (+) MS m/e=314 (MH⁺), ESI (−) MS m/e=312 (MH⁻).

### COMPOUND 38: 2-(5-CHLORO-1-ETHYL-1,3,4,9-TETRAHYDRO-PYRANO[3,4-B]INDOL-1-YL)-ETHANOL

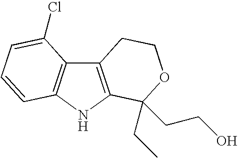

### 38.A. Synthesis of (4-Chloro-1H-indol-3-yl)-oxo-acetic acid ethyl ester

Following the procedure of example 36.C. except using 4-chloro-1H-indole as the indole component afforded the title compound as a solid.

### 38.B. Synthesis of 5-Chloro-1-ethyl-1,3,4,9-tetrahydro-pyrano[3,4-b]indol-1-yl)-acetic acid ethyl ester

Following the procedure of example 36.D. except using (4-chloro-1H-indol-3-yl)-oxo-acetic acid ethyl ester as the ester component afforded the title compound as an oil. ¹H NMR (008-08) MS. ESI (+) MS m/e=322 (MH⁺), ESI (−) MS m/e=320 (MH⁻).

### 38.C. Synthesis of (5-Chloro-1-ethyl-1,3,4,9-tetrahydro-pyrano[3,4-b]indol-1-yl)-acetic acid

Following the procedure of example 22.B except using (5-chloro-1-ethyl-1,3,4,9-tetrahydro-pyrano[3,4-b]indol-1-yl)-acetic acid ethyl ester as the ester component afforded the title compound as a solid. ESI (+) MS m/e=294 (MH⁺), ESI (−) MS m/e=292 (MH⁻).

### 38.D. Synthesis of 2-(5-Chloro-1-ethyl-1,3,4,9-tetrahydro-pyrano[3,4-b]indol-1-yl)-ethanol

Following the procedure of example 22.C. except using (5-chloro-1-ethyl-1,3,4,9-tetrahydro-pyrano[3,4-b]indol-1-yl)-acetic acid as the carboxylic acid component afforded the title compound as a solid. ¹H NMR (500 MHz, CDCl₃) δ 8.04 (br, 1H), 7.20 (dd, 1H), 7.04 (m, 2H), 4.05 (m, 1H), 3.97 (m, 1H), 3.68 (m, 2H), 3.16 (m, 2H), 2.19 (m, 1H), 2.04 (m, 1H), 1.98 (m, 1H), 1.89 (m, 1H), 0.92 (t, 3H); ESI (−) MS m/e=278 (MH⁻).

### COMPOUND 39: 2-(1-ETHYL-5-FLUORO-1,3,4,9-TETRAHYDRO-PYRANO[3,4-B]INDOL-1-YL)-ETHANOL

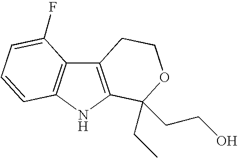

### 39.A. Synthesis of (4-Fluoro-1H-indol-3-yl)-oxo-acetic acid ethyl ester

Following the procedure of example 36.C. except using 4-fluoro-1H-indole as the indole component afforded the title compound as a solid.

### 39.B. Synthesis of (1-Ethyl-5-fluoro-1,3,4,9-tetrahydro-pyrano[3,4-b]indol-1-yl)-acetic acid ethyl ester

Following the procedure of example 36.D. except using (4-fluoro-1H-indol-3-yl)-oxo-acetic acid ethyl ester as the ester component afforded the title compound as an oil. ESI (−) MS m/e=304 (MH⁻).

### 39.C. (1-Ethyl-5-fluoro-1,3,4,9-tetrahydro-pyrano[3,4-b]indol-1-yl)-acetic acid

Following the procedure of example 22.B. except using (1-ethyl-5-fluoro-1,3,4,9-tetrahydro-pyrano[3,4-b]indol-1-yl)-acetic acid ethyl ester as the ester component afforded the title compound as a solid. ¹H NMR (500 MHz, CDCl₃) δ 9.70 (br, 1H), 8.75 (br, 1H), 7.06 (m, 2H), 6.73 (dd, 1H), 4.08 (m, 1H), 4.04 (m, 1H), 3.00 (m, 4H), 2.10 (m, 1H), 2.01 (m, 1H), 0.86 (t, 3H); ESI (+) MS m/e=278 (MH⁺), ESI (−) MS m/e=276 (MH⁻).

### 39.D. Synthesis of 2-(1-Ethyl-5-fluoro-1,3,4,9-tetrahydro-pyrano[3,4-b]indol-1-yl)-ethanol

Following the procedure of example 22.C. except using (1-ethyl-5-fluoro-1,3,4,9-tetrahydro-pyrano[3,4-b]indol-1-yl)-acetic acid as the carboxylic acid component afforded the title compound as a solid. ¹H NMR (500 MHz, CDCl₃) δ 7.97 (br, 1H), 7.08 (d, 1H), 7.04 (ddd, 1H), 6.75 (dd, 1H), 4.04 (m, 1H), 3.98 (m, 1H), 3.67 (m, 2H), 3.04 (m, 1H), 2.93 (m, 1H), 2.61 (br, 1H), 2.19 (m, 1H), 2.03 (m, 1H), 2.01 (m, 1H), 1.89 (m, 1H), 0.92 (t, 3H); ESI (+) MS m/e=264 (MH⁺), ESI (−) MS m/e=262 (MH⁻).

### COMPOUND 40: 2-(1-ETHYL-6-FLUORO-1,3,4,9-TETRAHYDRO-PYRANO[3,4-B]INDOL-1-YL)-ETHANOL

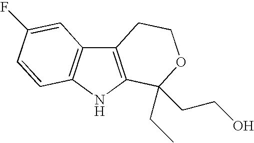

### 40.A. Synthesis of (5-Fluoro-1H-indol-3-yl)-oxo-acetic acid ethyl ester

Following the procedure of example 36.C. except using 5-fluoro-1H-indole as the indole component afforded the title compound as a solid.

### 40.B. Synthesis of (1-Ethyl-6-fluoro-1,3,4,9-tetrahydro-pyrano[3,4-b]indol-1-yl)-acetic acid ethyl ester

Following the procedure of example 36.D. except using (5-fluoro-1H-indol-3-yl)-oxo-acetic acid ethyl ester as the ester component afforded the title compound as an oil.

### 40.C. Synthesis of 2-(1-Ethyl-6-fluoro-1,3,4,9-tetrahydro-pyrano[3,4-b]indol-1-yl)-ethanol

Following the procedure of example 22.C. except using (1-ethyl-6-fluoro-1,3,4,9-tetrahydro-pyrano[3,4-b]indol-1-yl)-acetic acid ethyl ester as the carboxylic acid component afforded the title compound as a solid. ¹H NMR (500 MHz, CDCl₃) δ 7.91 (br, 1H), 7.22 (dd, 1H), 7.14 (dd, 1H), 6.90 (ddd, 1H), 4.05 (m, 1H), 4.00 (m, 1H), 3.68 (m, 2H), 2.71 (dt, 1H), 2.69 (br, 1H), 2.18 (m, 1H), 2.04 (m, 1H), 1.97 (m, 1H), 1.89 (m, 1H), 0.92 (t, 3H); ESI (+) MS m/e=264 (MH⁺), ESI (−) MS m/e=262 (MH⁻).

### COMPOUND 41: 2-(6-CHLORO-1-ETHYL-1,3,4,9-TETRAHYDRO-PYRANO[3,4-B]INDOL-1-YL)-ETHANOL

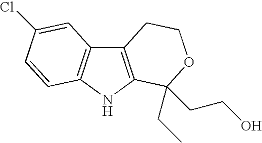

### 41.A. Synthesis of (5-Chloro-1H-indol-3-yl)-oxo-acetic acid ethyl ester

Following the procedure of example 36.C. except using 5-chloro-1H-indole as the indole component afforded the title compound as a solid. ¹H NMR (500 MHz, DMSO-d₆) δ 12.54 (br, 1H), 8.50 (d, 1H), 8.12 (d, 1H), 7.56 (d, 1H), 7.30 (dd, 1H), 4.36 (q, 2H), 2.48 (t, br, 1H), 1.32 (t, 3H); APCI (−) MS m/e=250 (MH⁻).

### 41.B. Synthesis of (6-Chloro-1-ethyl-1,3,4,9-tetrahydro-pyrano[3,4-b]indol-1-yl)-acetic acid ethyl ester

Following the procedure of example 36.D. except using (5-chloro-1H-indol-3-yl)-oxo-acetic acid ethyl ester as the ester component afforded the title compound as an oil. ¹H NMR (500 MHz, CDCl₃) δ 9.18 (br, 1H), 7.46 (d, 1H), 7.26 (d, 1H), 7.11 (dd, 1H), 4.19 (m, 2H), 4.03 (m, 1H), 3.93 (m, 1H), 2.99 (d, 1H), 2.90 (d, 1H), 2.77 (m, 1H), 2.72 (m, 1H), 2.11 (m, 1H), 1.98 (m, 1H), 1.27 (t, 3H), 0.81 (t, 3H); APCI (+) MS m/e=322 (MH⁺), APCI (−) MS m/e=320(MH⁻).

### 41. C Synthesis of (6-Chloro-1-ethyl-1,3,4,9-tetrahydro-pyrano[3,4-b]indol-1-yl)-acetic acid

Following the procedure of example 22.B. except using (6-chloro-1-ethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 9.50 (br, 1H), 8.68 (br, 1H), 7.46 (s, 1H), 7.23 (d, 1H), 7.12 (d, 1H), 4.09 (m, 1H), 4.03 (m, 1H), 3.03 (d, 1H), 2.99 (d, 1H), 2.79 (m, 2H), 2.10 (m, 1H), 2.01 (m, 1H), 0.86 (t, 3H); ESI (+) MS m/e=294 (MH⁺), ESI (−) MS m/e=292 (MH⁻).

### 41.D. Synthesis of 2-(6-Chloro-1-ethyl-1,3,4,9-tetrahydro-pyrano[3,4-b]indol-1-yl)-ethanol

Following the procedure of example 22.C. except using (6-chloro-1-ethyl-1,3,4,9-tetrahydro-pyrano[3,4-b]indol-1-yl)-acetic acid as the carboxylic acid component afforded the title compound as a solid. ¹H NMR (500 MHz, CDCl₃) δ 8.01 (br, 1H), 7.46 (s, 1H), 7.22 (d, 1H), 7.12 (d, 1H), 4.05 (m, 1H), 3.99 (m, 1H), 3.67 (m, 2H), 2.83 (m, 1H), 2.72 (m, 1H), 2.65 (br, 1H), 2.19 (m, 1H), 2.00 (m, 2H), 1.88 (m, 1H), 0.92 (t, 3H); APCI (+) MS m/e=280 (MH⁺), APCI (−) MS m/e=278 (MH⁻).

### COMPOUND 42: 2-(6-BROMO-1-ETHYL-1,3,4,9-TETRAHYDRO-PYRANO[3,4-B]INDOL-1-YL)-ETHANOL

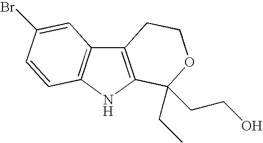

### 42.A. Synthesis of (5-Bromo-1H-indol-3-yl)-oxo-acetic acid ethyl ester

Following the procedure of example 36, step (c) except using 5-bromo-1H-indole as the indole component afforded the title compound as a solid.

### 42.B. Synthesis of (6-Bromo-1-ethyl-1,3,4,9-tetrahydro-pyrano[3,4-b]indol-1-yl)-acetic acid ethyl ester

Following the procedure of example 36.B. except using (5-bromo-1H-indol-3-yl)-oxo-acetic acid ethyl ester as the ester component afforded the title compound as an oil.

### 42.C. Synthesis of 2-(6-Bromo-1-ethyl-1,3,4,9-tetrahydro-pyrano[3,4-b]indol-1-yl)-ethanol

Following the procedure of example 28.C. except using (6-bromo-1-ethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 7.95 (br, 1H), 7.62 (d, 1H), 7.24 (dd, 1H), 7.19 (d, 1H), 4.05 (m, 1H), 3.97 (m, 1H), 3.67 (m, 2H), 2.84 (m, 1H), 2.71 (m, 1H), 2.55 (br, 1H), 2.19 (m, 1H), 2.03 (m, 1H), 1.97 (m, 1H), 1.88 (m, 1H), 0.91 (t, 3H); ESI (+) MS m/e=324 (MH⁺), ESI (−) MS m/e=322 (MH⁻).

### COMPOUND 43: 2-(1-ETHYL-7-FLUORO-1,3,4,9-TETRAHYDRO-PYRANO[3,4-B]INDOL-1-YL)-ETHANOL

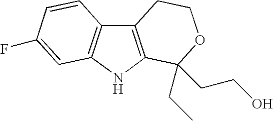

### 43.A. Synthesis of (6-Fluoro-1H-indol-3-yl)-oxo-acetic acid ethyl ester

Following the procedure of example 36.C. except using 6-fluoro-1H-indole as the indole component afforded the title compound as a solid. ¹H NMR (500 MHz, CDCl₃) δ 8.75 (br, 1H), 8.48 (d, 1H), 8.39 (dd, 1H), 7.12 (m, 2H), 4.41 (q, 2H), 1.43 (t, 3H); ESI (−) MS m/e=234(MH⁻).

### 43.B. Synthesis of (1-Ethyl-7-fluoro-1,3,4,9-tetrahydro-pyrano[3,4-b]indol-1-yl)-acetic acid ethyl ester

Following the procedure of example 36.D. except using (6-fluoro-1H-indol-3-yl)-oxo-acetic acid ethyl ester as the ester component afforded the title compound as an oil.

### 43.C. Synthesis of (1-Ethyl-7-fluoro-1,3,4,9-tetrahydro-pyrano[3,4-b]indol-1-yl)-acetic acid

Following the procedure of example 22.B. except using (1-ethyl-7-fluoro-1,3,4,9-tetrahydro-pyrano[3,4-b]indol-1-yl)-acetic acid ethyl ester as the ester component afforded the title compound as a solid. ¹H NMR (500 MHz, CDCl₃) δ 11.8 (br, 1H), 9.53 (br, 1H), 7.29 (dd, 1H), 6.93 (dd, 1H), 6.74 (ddd, 1H), 3.94 (m, 1H), 3.89 (m, 1H), 2.86 (d, 1H), 2.82 (d, 1H), 2.69 (m, 1H), 2.66 (m, 1H), 2.03 (m, 1H), 1.95 (m, 1H), 0.74 (t, 3H); ESI (+) MS m/e=278 (MH⁺), ESI (−) MS m/e=276 (MH⁻).

### 43.D. Synthesis of 2-(1-Ethyl-7-fluoro-1,3,4,9-tetrahydro-pyrano[3,4-b]indol-1-yl)-ethanol

Following the procedure of example 22.C. except using (1-ethyl-7-fluoro-1,3,4,9-tetrahydro-pyrano[3,4-b]indol-1-yl)-acetic acid as the carboxylic acid component afforded the title compound as a solid. ¹H NMR (500 MHz, CDCl₃) δ 7.95 (br, 1H), 7.40 (dd, 1H), 7.00 (dd, 1H), 6.88 (ddd, 1H), 4.06 (m, 1H), 3.99 (m, 1H), 3.65 (m, 2H), 2.85 (m, 1H), 2.71 (m, br, 2H), 2.18 (m, 1H), 2.02 (m, 1H), 1.98 (m, 1H), 1.88 (m, 1H), 0.92 (t, 3H); ESI (+) MS m/e=264 (MH⁺), ESI (−) MS m/e=262 (MH⁻).

### COMPOUND 44: 2-(7-CHLORO-1-ETHYL-1,3,4,9-TETRAHYDRO-PYRANO[3,4-B]INDOL-1-YL)-ETHANOL

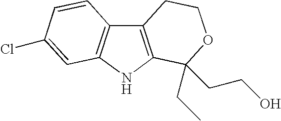

### 44.A. Synthesis of (6-Chloro-1H-indol-3-yl)-oxo-acetic acid ethyl ester

Following the procedure of example 36.c. except using 6-chloro-1H-indole as the indole component afforded the title compound as a solid. ¹H NMR (500 MHz, CDCl₃) δ 11.50 (br, 1H), 8.30 (d, 1H), 8.21 (d, 1H), 7.37 (d, 1H), 7.15 (dd, 1H), 4.31 (q, 2H), 1.33 (t, 3H); ESI (+) MS m/e=252 (MH⁺), ESI (−) MS m/e=250 (MH⁻).

### 44.B. Synthesis of (7-Chloro-1-ethyl-1,3,4,9-tetrahydro-pyrano[3,4-b]indol-1-yl)-acetic acid ethyl ester

Following the procedure of example 22.D. except using (6-chloro-1H-indol-3-yl)-oxo-acetic acid ethyl ester as the ester component afforded the title compound as an oil.

### 44.C. Synthesis of (7-Chloro-1-ethyl-1,3,4,9-tetrahydro-pyrano[3,4-b]indol-1-yl)-acetic acid

Following the procedure of example 22.B. except using (7-chloro-1-ethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 11.80 (br, 1H), 9.57 (br, 1H), 7.30 (d, 1H), 7.24 (d, 1H), 6.95 (dd, 1H), 3.97 (m, 1H), 3.88 (m, 1H), 2.88 (d, 1H), 2.80 (d, 1H), 2.71 (m, 1H), 2.66 (dt, 1H), 2.04 (m, 1H), 1.96 (m, 1H), 0.74 (t, 3H); ESI (+) MS m/e=294 (MH⁺), ESI (−) MS m/e=292 (MH⁻).

### 44.D. Synthesis of 2-(7-Chloro-1-ethyl-1,3,4,9-tetrahydro-pyrano[3,4-b]indol-1-yl)-ethanol

Following the procedure of example 22.C. except using (7-chloro-1-ethyl-1,3,4,9-tetrahydro-pyrano[3,4-b]indol-1-yl)-acetic acid as the carboxylic acid component afforded the title compound as a solid. ¹H NMR (500 MHz, CDCl₃) δ 8.02 (br, 1H), 7.40 (d, 1H), 7.30 (d, 1H), 7.08 (dd, 1H), 4.05 (m, 1H), 3.99 (m, 1H), 3.66 (m, 2H), 2.73 (dt, 1H), 2.71 (br, 1H), 2.11 (m, 1H), 2.02 (m, 1H), 1.96 (m, 1H), 1.88 (m, 1H), 0.91 (t, 3H); ESI (+) MS m/e=280 (MH⁺), ESI (−) MS m/e=278 (MH⁻).

### COMPOUND 45: 2-(1-ETHYL-6,8-DIMETHYL-1,3,4,9-TETRAHYDRO-PYRANO[3,4-B]INDOL-1-YL)-ETHANOL

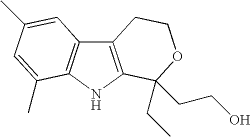

### 45.A. Synthesis of 5,7-Dimethyl-1H-indole

To solution of 5,7-dimethyl-1H-indole-2,3-dione in tetrahydrofuran at 0° C. was added 1.0 M solution of borane-tetrahydrofuran complex in tetrahydrofuran (40 mL). After stirred at room temperature overnight, a 5% HCl solution was added to the mixture and it was stirred 20 minutes. It was neutralized with saturated sodium bicarbonate solution and extracted with ethyl acetate. Extracts were dried over magnesium sulfate and evaporated to dryness to afford the title compound as oil.

### 45.B. Synthesis of (5,7-Dimethyl-1H-indol-3-yl)-oxo-acetic acid ethyl ester

Following the procedure of example 36.C. except using 5,7-dimethyl-1H-indole as the indole component afforded the title compound as a solid. ESI (+) MS m/e=246 (MH⁺).

### 45.C. Synthesis of (1-Ethyl-6,8-dimethyl-1,3,4,9-tetrahydro-pyrano[3,4-b]indol-1-yl)-acetic acid ethyl ester

Following the procedure of example 36.D. except using (5,7-dimethyl-1H-indol-3-yl)-oxo-acetic acid ethyl ester component afforded the title compound as an oil.

### 45.D. Synthesis of (1-Ethyl-6,8-dimethyl-1,3,4,9-tetrahydro-pyrano[3,4-b]indol-1-yl)-acetic acid

Following the procedure of example 22.B. except using (1-ethyl-6,8-dimethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 9.50 (br, 1H), 8.28 (br, 1H), 7.14 (s, 1H), 6.82 (s, 1H), 4.10 (m, 1H), 4.06 (m, 1H), 3.02 (d, 2H), 3.01 (d, 1H), 2.81 (m, 2H), 2.41 (s, 3H), 2.40 (s, 3H), 2.10 (m, 1H), 2.03 (m, 1H), 0.87 (t, 3H); ESI (+) MS m/e=288 (MH⁺), ESI (−) MS m/e=286 (MH⁻).

### 45. E Synthesis of 2-(1-Ethyl-6,8-dimethyl-1,3,4,9-tetrahydro-pyrano[3,4-b]indol-1-yl)-ethanol

Following the procedure of example 22.C. except using (1-ethyl-6,8-dimethyl-1,3,4,9-tetrahydro-pyrano[3,4-b]indol-1-yl)-acetic acid as the carboxylic acid component afforded the title compound as a solid. ¹H NMR (500 MHz, CDCl₃) δ 7.51 (br, 1H), 7.15 (s, 1H), 6.83 (s, 1H), 4.06 (m, 1H), 3.98 (m, 1H), 3.67 (m, 2H), 2.85 (m, 1H), 2.72 (dt, 1H), 2.69 (br, 1H), 2.43 (s, 3H), 2.42 (s, 3H), 2.20 (m, 1H), 2.06 (m, 1H), 2.03 (m, 1H), 1.89 (m, 1H), 0.95 (t, 3H); ESI (+) MS m/e=274 (MH⁺), ESI (−) MS m/e=272 (MH⁻).

### COMPOUND 46: 2-(6,8-DICHLORO-1-ETHYL-1,3,4,9-TETRAHYDRO-PYRANO[3,4-B]INDOL-1-YL)-ETHANOL

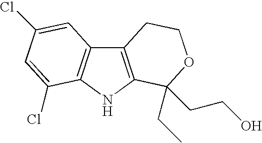

### 46.A. Synthesis of 5,7-Dichloro-1H-indole

Following the procedure of example 45.A. except using 5,7-dichloro-1H-indole-2,3-dione as the dione component afforded the title compound as a oil.

### 46.B. Synthesis of (5,7-Dichloro-1H-indol-3-yl)-oxo-acetic acid ethyl ester

Following the procedure of example 36.C. except using 5,7-dichloro-1H-indole as the indole component afforded the title compound as a solid.

### 46.C. Synthesis of (6,8-Dichloro-1-ethyl-1,3,4,9-tetrahydro-pyrano[3,4-b]indol-1-yl)-acetic acid ethyl ester

Following the procedure of example 36.D. except using (5,7-dichloro-1H-indol-3-yl)-oxo-acetic acid ethyl ester component afforded the title compound as an oil.

### 46.D. Synthesis of (6,8-Dichloro-1-ethyl-1,3,4,9-tetrahydro-pyrano[3,4-b]indol-1-yl)-acetic acid

Following the procedure of example 22.B. except using (6,8-dichloro-1-ethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 9.07 (br, 1H), 7.03 (d, 1H), 6.97 (d, 1H), 4.07 (m, 1H), 4.01 (m, 1H), 3.15 (t, 2H), 3.10 (d, 1H), 3.03 (d, 1H), 2.15 (m, 1H), 2.05 (m, 1H), 0.88 (t, 3H); ESI (+) MS m/e=328 (MH⁺), ESI (−) MS m/e=326 (MH⁻).

### 46.E. Synthesis of 2-(6,8-Dichloro-1-ethyl-1,3,4,9-tetrahydro-pyrano[3,4-b]indol-1-yl)-ethanol

Following the procedure of example 22.C. except using (6,8-dichloro-1-ethyl-1,3,4,9-tetrahydro-pyrano[3,4-b]indol-1-yl)-acetic acid as the carboxylic acid component afforded the title compound as a solid. ¹H NMR (500 MHz, CDCl₃) δ 8.25 (br, 1H), 7.03 (d, 1H), 6.98 (d, 1H), 4.03 (m, 1H), 3.99 (m, 1H), 3.71 (m, 2H), 3.13 (m, 2H), 2.57 (br, 1H), 2.23 (m, 1H), 2.07 (m, 1H), 2.04 (m, 1H), 1.92 (m, 1H), 0.93 (t, 3H); ESI (+) MS m/e=314 (MH⁺), ESI (−) MS m/e=312 (MH⁻).

### COMPOUND 47: 2-(6-BROMO-1,8-DIETHYL-1,3,4,9-TETRAHYDRO-PYRANO[3,4-B]INDOL-1-YL)-ETHANOL

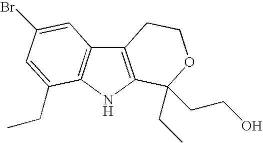

### 47.A. Synthesis of N-(4-Bromo-2-ethyl-phenyl)-2-hydroxyimino-acetamide

Following the procedure of example 36.A. except using 4-bromo-2-ethylaniline as the aniline component afforded the title compound as a solid. ESI (−) MS m/e=269 (MH⁻).

### 47.B. Synthesis of 5-Bromo-7-ethyl-1H-indole-2,3-dione

Following the procedure of example 36.B. except using N-(4-Bromo-2-ethyl-phenyl)-2-hydroxyimino-acetamide as the acetamide component afforded the title compound as a solid. ESI (−) MS m/e=252 (MH⁻).

### 47.C. Synthesis of (5-Bromo-7-ethyl-1H-indol-3-yl)-oxo-acetic acid ethyl ester

Following the procedure of example 36.C. except using 5-Bromo-7-ethyl-1H-indole-2,3-dione as the dione component afforded the title compound as a solid. ESI (+) MS m/e=324 (MH⁺), ESI (−) MS m/e=322 (MH⁻).

### 47.D. Synthesis of (6-Bromo-1,8-diethyl-1,3,4,9-tetrahydro-pyrano[3,4-b]indol-1-yl)-acetic acid ethyl ester

Following the procedure of example 36.D. except using (5-bromo-7-ethyl-1H-indol-3-yl)-oxo-acetic acid ethyl ester as the ester component afforded the title compound as a solid.

### 47.E. Synthesis of 2-(6-Bromo-1,8-diethyl-1,3,4,9-tetrahydro-pyrano[3,4-b]indol-1-yl)-ethanol

Following the procedure of example 28.C. except using (6-bromo-1,8-diethyl-1,3,4,9-tetrahydro-pyrano[3,4-b]indol-1-yl)-acetic acid ethyl ester as the ester component afforded the title compound as a solid. ¹H NMR (500 MHz, CDCl₃) δ 7.89 (br, 1H), 7.48 (d, 1H), 7.11 (d, 1H), 4.05 (m, 1H), 3.99 (m, 1H), 3.70 (m, 2H), 2.80 (m, 3H), 2.71 (dt, 1H), 2.55 (br, t, 1H), 2.19 (m, 1H), 2.05 (m, 1H), 2.01 (m, 1H), 1.90 (m, 1H), 1.33 (t, 3H), 0.92 (t, 3H); ESI (+) MS m/e=352 (MH⁺), ESI (−) MS m/e=350 (MH⁻).

### COMPOUND 48: 2-(1,8-DIETHYL-1,3,4,9-TETRAHYDRO-PYRANO[3,4-B]INDOL-1-YL)-N,N-DIMETHYL-ACETAMIDE

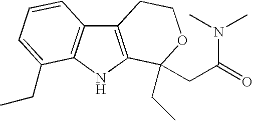

2-(1,8-Diethyl-1,3,4,9-tetrahydro-pyrano[3,4-b]indol-1-yl)-N,N-dimethyl-acetamide. Following the procedure of example 27 except using dimethylamine as the amine component afforded the title compound as a solid. ¹H NMR (500 MHz, CDCl₃) δ 9.39 (br, 1H), 7.35 (d, 1H), 7.05 (t, 1H), 6.99 (d, 1H), 6.19 (m, 1H), 4.06 (m, 1H), 3.98 (m, 1H), 2.84 (s, m, 9H), 2.11 (m, 1H), 2.01 (m, 1H), 1.36 (t, 3H), 0.85 (t, 3H); ESI (−) MS m/e=299 (MH⁻).

### COMPOUND 49: 2-(9-BENZYL-1,8-DIETHYL-1,3,4,9-TETRAHYDRO-PYRANO[3,4-B]INDOL-1-YL)-ETHANOL

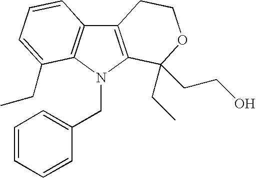 Compound 49 was synthesized according to the following scheme:

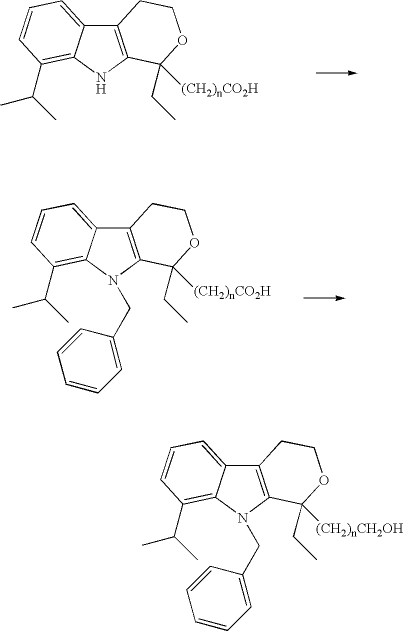

### 49.A. Synthesis of (9-Benzyl-1,8-diethyl-1,3,4,9-tetrahydro-pyrano[3,4-b]indol-1-yl)-acetic acid

To a solution of (1,8-diethyl-1,3,4,9-tetrahydro-pyrano[3,4-b]indol-1-yl)-acetic acid (0.51 g, 1.8 mmol) in tetrahydrofuran at room temperature was added sodium hydride (60% dispersion in mineral oil, 0.4 g). After being heated at 50° C. for 2 hours, benzyl bromide (0.6 g, 3.5 mmol) was added and the solution was stirred for another 2 hours. It was quenched with ethyl acetate and washed with water. The ethyl acetate layer was dried over magnesium sulfate and evaporated to dryness. Flash chromatography on silica gel provided 0.486 g (73%) of the title compound as a solid. ¹H NMR (500 MHz, CDCl₃) δ 7.13 (m, 3H), 6.97 (d, 1H), 6.74 (d, 1H), 6.68 (t, 1H), 6.21 (d, 1H), 3.90 (s, 1H), 3.63 (m, 1H), 3.35 (td, 1H), 3.18 (d, 1H), 3.00 (d, 1H), 2.67 (q, 2H), 2.44 (q, 2H), 2.10 (m, 1H), 1.85 (d, 1H), 1.52 (m, 1H), 1.41 (m, 1H), 1.16 (t, 3H), 0.75 (t, 3H); ESI (+) MS m/e=278 (MH⁺).

### 49.B. Synthesis of 2-(9-Benzyl-1,8-diethyl-1,3,4,9-tetrahydro-pyrano[3,4-b]indol-1-yl)-ethanol

To a solution of (9-benzyl-1,8-diethyl-1,3,4,9-tetrahydro-pyrano[3,4-b]indol-1-yl)-acetic acid (0.45 g, 1.2 mmol) in tetrahydrofuran at room temperature was added 1.0 M solution of borane-tetrahydrofuran complex in tetrahydrofuran and it was stirred at 90° C. for 4 hours. The mixture was quenched with 5% HCl solution and stirred at room temperature for 20 minutes. It was extracted with ethyl acetate and washed with saturated sodium bicarbonate. The extracts were dried over magnesium sulfate and evaporated to dryness. Flash chromatography on silica gel provided 0.321 g (74%) of the title compound as a solid. ¹H NMR (500 MHz, CDCl₃) δ 7.17 (m, 3H), 6.94 (d, 1H), 6.84 (m, 2H), 6.70 (t, 1H), 6.56 (d, 1H), 3.87 (m, 1H), 3.79 (m, 1H), 3.68 (dt, 1H), 3.64 (br, 1H), 3.41 (td, 1H), 2.93 (q, 2H), 2.43 (q, 2H), 2.04 (m, 1H), 1.93 (dt, 1H), 1.86 (m, 1H), 1.77 (m, 1H), 1.49 (m, 1H), 1.38 (m, 1H), 1.17 (t, 3H), 0.70 (t, 3H).

### COMPOUND 50: 2-(1,8-DIETHYL-9-METHYL-1,3,4,9-TETRAHYDRO-PYRANO[3,4-B]INDOL-1-YL)-ETHANOL

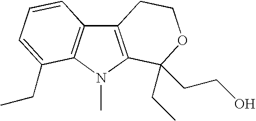

### 50.A. Synthesis of 2-(1,8-Diethyl-9-methyl-1,3,4,9-tetrahydro-pyrano[3,4-b]indol-1-yl)-acetic acid

Following the procedure of example 49.A. except using (1,8-Diethyl-1,3,4,9-tetrahydro-pyrano[3,4-b]indol-1-yl)-acetic acid as the indole component afforded the title compound as an oil.

### 50.B. Synthesis of 2-(1,8-Diethyl-9-methyl-1,3,4,9-tetrahydro-pyrano[3,4-b]indol-1-yl)-ethanol

Following the procedure of example 49.B. except using 2-(1,8-diethyl-9-methyl-1,3,4,9-tetrahydro-pyrano[3,4-b]indol-1-yl)-acetic acid as the carboxylic acid component afforded the title compound as a solid. ¹H NMR (500 MHz, CDCl₃) δ 7.35 (dd, 1H), 7.03 (t, 1H), 6.98 (d, 1H), 4.04 (m, 1H), 3.93 (m, 1H), 3.91 (s, 3H), 3.75 (m, 1H), 3.63 (m, 1H), 3.11 (q, 2H), 2.87 (m, 1H), 2.76 (dt, 1H), 2.68 (br, 1H), 2.27 (m, 1H), 2.22 (m, 1H), 2.12 (m, 1H), 1.97 (m, 1H), 1.35 (t, 3H), 0.94 (t, 3H); ESI (+) MS m/e=288 (MH⁺).

### COMPOUND 51: 2-(7-BROMO-1,8-DIETHYL-1,3,4,9-TETRAHYDROPYRANO[3,4-B]INDOL-1-YL)ETHANOL

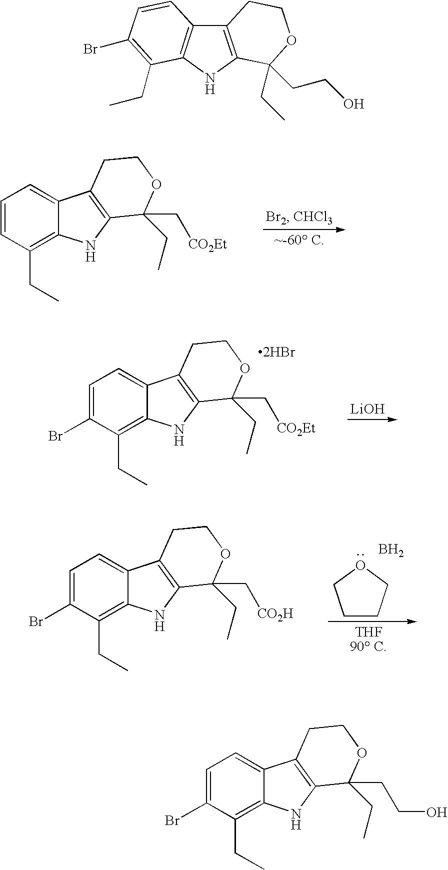

### 51.A. Synthesis of Ethyl 2-(7-bromo-1,8-diethyl-1,3,4,9-tetrahydropyrano[3,4-b]indol-1-yl)acetate.

(R,S)-Etodolac ethyl ester (5 g, 15.8 mmol) was dissolved in chloroform (50 ml) and cooled to −60° C. with a dry ice/acetone bath. To this solution was added dropwise a solution of bromine (2.53 g, 15.8 mmol) in chloroform (50 ml) during 2 hr. After the addition, the reaction mixture was allowed to warm to −20° C. and triethylamine (5 ml) was added dropwise followed by silica gel (˜20 g). The mixture was stirred for 10 min, filtered through silica gel (˜10 g), and the filtrate evaporated to dryness. The crude product was recrystallized in hexane/dichloromethane (60 ml/20 ml) to give (4.5 g, 72%) of product. ¹H NMR (300 MHz, CDCl₃) δ 9.23 (b, NH), 7.45 (d, 1H), 7.15 (d, 1H), 4.21 (qrt, 2H), 4.15 (m, 1H), 3.95 (m, 1H), 3.25 (dd, 2H), 2.94 (m, 2H), 2.25 (m, 1H), 2.1 (m, 1H), 1.45 (t, 3H), 1.15 (t, 3H), 0.85 (t, 3H).

### 51.B. Synthesis of 2-(7-Bromo-1,8-diethyl-1,3,4,9-tetrahydropyrano[3,4-b]indol-1-yl)acetic acid

To a stirred solution of ethyl 2-(7-bromo-1,8-diethyl-1,3,4,9-tetrahydropyrano[3,4-b]indol-1-yl)acetate (2.8 g, 5 mmol) in dioxane (40 ml) was added lithium hydroxide monohydrate (2.8 g, 67 mmol) and water (30 ml). The mixture was stirred at room temperature overnight. It was concentrated under reduced pressure, neutralized with 5% HCl, extracted with CH₂Cl₂, dried over MgSO₄, and concentrated. The crude product was recrystallized in dichloromethane/hexane (60 ml/20 ml) to give a white solid (980 mg, 53%). ¹H NMR (300 MHz, CDCl₃) δ 8.68 (br, NH)), 7.27 (d, 1H), 7.19 (d, 1H), 4.06 (m, 2H), 3.04 (qrt, 2H), 2.95 (qrt, 2H), 2.80 (m, 2H), 2.09 (m, 2H), 1.24 (t, 3H), 0.874 (t, 3H).

### 51.C. Synthesis of 2-(7-Bromo-1,8-diethyl-1,3,4,9-tetrahydropyrano[3,4-b]indol-1-yl)ethanol

To a stirred solution of 2-(7-Bromo-1,8-diethyl-1,3,4,9-tetrahydropyrano[3,4-b]indol-1-yl)acetic acid (0.87 g, 2.4 mmol) in THF (5 ml), was added dropwise via syringe borane-tetrahydrofuran complex, 1.0 M solution in tetrahydrofuran (3.6 ml, 3.6 mmol) during 30 min. The mixture was stirred at 90° C. for 8 hr, cooled, quenched with distilled water and 5% HCl, extracted with EtOAc. The organic phases collected, washed with brine, dried over MgSO₄, and evaporated to give a residue which was chromatographed on silica gel. Elution with hexane-EtOAc (1:1) gave the product which was further recrystallized in hexane/dichloromethane to give the product (0.64 g, 76%). ¹H NMR (300 MHz, CDCl₃) δ 7.91 (b, NH), 7.28 (d, 1H), 7.20 (d, 1H), 4.02 (m, 2H), 3.71 (m, 2H), 2.95 (qrt, 1H), 2.81 (m, 1H), 2.75 (t, 1H), 2.69 (t, 1H), 2.58 (t, 1H), 2.19 (m, 1H), 2.04 (m, 2H), 1.26(t, 3H), 0.93 (t, 3H).

### COMPOUND 54: 2-(6-BROMO-1-ETHYL-1,3,4,9-TETRAHYDRO-8-ISOPROPYLPYRANO[3,4-B]INDOL-1-YL)ETHANOL

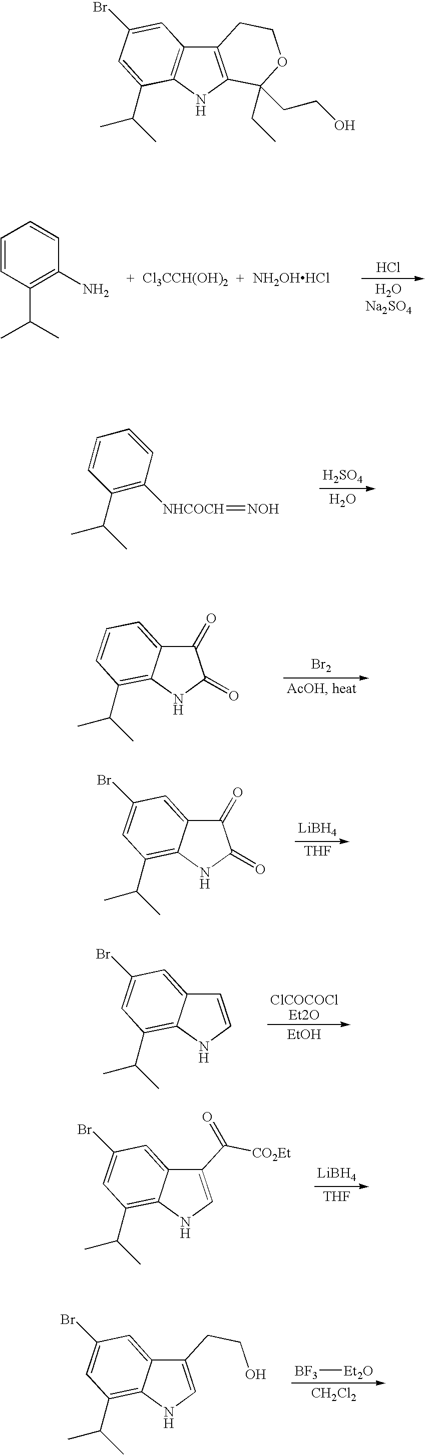

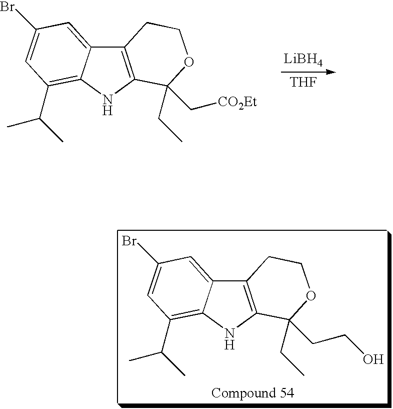

### 54.A. Synthesis of 2-(Hydroxyimino)-N-(2-isopropylphenyl)acetamide

In a 2-1 round-bottomed flask are placed water (1000 ml), followed by chloral hydrate (49 g, 0.30 mol), anhydrous sodium sulfate (225 g), 2-isopropylaniline (50 g, 0.37 mol), concentrated hydrochloric acid (22 ml, 0.26 mol), hydroxylamine hydrochloride (57 g, 0.81 mol). The solution was boiled for 3 hr, cooled, quenched with water, and extracted with ethyl acetate. The extracts were dried over MgSO₄, and evaporated. The residue was purified by elution from a silica gel column with hexane/EtOAc (7:3) to afford the product (26.7 g, 35%). ¹H NMR (300 MHz, CDCl₃) δ 8.28 (br, NH), 7.88 (dd, 1H), 7.82 (b, NOH), 7.63 (s, N═CH), 7.24 (m, 3H), 3.04 (m, 1H), 1.27 (d, 6H); ESI (+) MS m/e=207 (MH⁺), ESI (−) MS m/e=205 (MH⁻).

### 54.B. Synthesis of 7-Isopropylindoline-2,3-dione

To a stirred solution of concentrated H₂SO4 (210 ml) and H2O (50 ml), was added over 20 min (26.7 g, 0.13 mol) of 2-(hydroxyimino)-N-(2-isopropylphenyl)acetamide. The mixture was stirred at 75° C. for 2 hr, cooled and poured onto cracked ice. After standing for 15 min, it was extracted with EtOAc, washed with water, dried over MgSO4, and concentrated. Air drying afforded (23.8 g, 97%) of crude product). ¹H NMR (300 MHz, CDCl₃) δ 8.15 (b, NH), 7.49 (d, 2H), 7.11 (t, 1H), 2.87(m, 1H), 1.30 (d, 6H); ESI (+) MS m/e=190 (MH+), ESI (−) MS m/e=188 (MH−).

### 54.C. Synthesis of 5-Bromo-7-isopropylindoline-2,3-dione

7-isopropylindoline-2,3-dione (23.8 g, 0.12 mol) was added to a stirred solution of glacial acetic acid (700 ml). To this solution was added, via additional funnel bromine (7.8 ml, 0.15 mol) in glacial acetic acid (300 ml) during 30 min. After the addition, the combined mixture was stirred at 75° C. for 3 hr, cooled, and extracted with EtOAc. The organic extracts were washed with brine, dried over MgSO₄, and evaporated in vacuo; air dried to give (31.8 g, 94%) of crude product. ¹H NMR (300 MHz, CDCl₃) δ 8.04 (b, NH), 7.59 (dd 2H), 2.84 (m, 1H), 1.31 (d, 6H); ESI (+) MS m/e=269 (MH⁺), ESI (−) MS m/e=267 (MH⁻).

### 54.D. Synthesis of 5-Bromo-7-isopropyl-1H-indole

To a stirred solution of 5-bromo-7-isopropylindoline-2,3-dione (45.1 g, 0.17 mol) in THF (275 ml) at room temperature under a nitrogen atmosphere, was added, via syringe, 2.0 M solution of LiBH₄/THF (215 ml) over 30 min. The reaction mixture was stirred at 90° C. for 1 hr, cooled, quenched with distilled water and 5% HCl, and extracted with EtOAc. The extracts were washed with brine, dried over MgSO₄, and concentrated under reduced pressure. The crude product was purified by elution from a silica gel column with hexane/EtOAc (9:1) to give (14.5 g, 36%) of the product. ¹H NMR (300 MHz, CDCl₃) δ 8.18 (b, NH), 7.62 (d, 1H), 7.21 (t, 1H), 7.16 (d, 1H), 6.51 (dd, 1H), 3.20 (m, 1H), 1.38 (d, 6H); ESI (+) MS m/e=239 (MH+), ESI (−) MS m/e=237 (MH−).

### 54.E. Synthesis of Ethyl 2-(5-Bromo-7-isopropyl-1H-indol-3-yl)-2-oxoacetate

A 2.0 M solution of oxalyl dichloride in dichloromethane (60 ml, 0.12 mol) was added dropwise during 10 min to a solution of 5-Bromo-7-isopropyl-1H-indole (14.5 g, 0.061 mol) in Et₂O (220 ml) at room temperature under a nitrogen atmosphere. The mixture was stirred for 4.5 hr. The Et₂O was removed by evaporation and absolute EtOH (220 ml) was added. The resulting mixture was stirred at room temperature under a nitrogen atmosphere overnight. The EtOH was evaporated, and EtOAc was added to the residue and washed with sat. NaHCO₃ and brine. The organic layers were dried over MgSO₄, concentrated, and dried under vacuum to give a crude product (13.8 g, 67%). ¹H NMR (300 MHz, CDCl₃) δ 8.85 (b, NH), 8.46 (dd, 2H), 7.33 (d, 1H), 4.42 (qrt, 2H), 3.21 (m, 1H), 1.44 (t, 3H), 1.38 (d, 6H), ESI (+) MS m/e=339 (MH+), ESI (−) MS m/e=337 (MH−).

54. F Synthesis of 2-(5-Bromo-7-isopropyl-1H-indol-3-yl)ethanol. Ethyl 2-(5-bromo-7-isopropyl-1H-indol-3-yl)-2-oxoacetate (13.8 g, 0.04 mol) in THF (300 ml) was reduced with 2.0 M solution of LiBH4 in THF (50 ml, 0.1 mol) by refluxing under nitrogen atmosphere for 5 hr, cooled, quenched with distilled water and 5% HCl, and extracted with EtOAc. The extracts were washed with brine, dried over MgSO₄, and concentrated. The crude product was purified by eluting from silica gel with hexane/EtOAc to obtain (4.5 g, 39%) of product. ¹H NMR (300 MHz, CDCl₃) δ 8.05 (b, NH), 7.59 (d, 1H), 7.17 (d, 1H), 7.10 (d, 1H), 3.89 (t, 2H), 3.18 (m, 1H), 2.98 (t, 2H), 1.37 (d, 6H); ESI (+) MS m/e=283 (MH⁺), ESI (−) MS m/e=281 (MH⁻).

### 54. G. Synthesis of Ethyl 2-(6-Bromo-1-ethyl-1,3,4,9-tetrahydro-8-isopropylpyrano[3,4-b]indol-1-yl)acetate

To a suspension of 2-(5-bromo-7-isopropyl-1H-indol-3-yl)ethanol (4.5 g, 0.016 mol) under nitrogen atmosphere was added boron trifluoride diethyl etherate (2.2 ml, 0.18 mol), followed by dropwise addition of ethyl propionyl acetate (3.4 ml, 0.024 mol) over ten minutes. The mixture was stirred at room temperature for 1.5 hr. Dichloromethane was added to the mixture and the organic layer was washed with sat. NaHCO₃ and water, and dried over MgSO₄. The solvent was concentrated and air dried to give a crude product (6 g, 92%). ESI (+) MS m/e=409 (MH⁺), ESI (−) MS m/e=407 (MH⁻).

### 54. F Synthesis of 2-(6-Bromo-1-ethyl-1,3,4,9-tetrahydro-8-isopropylpyrano[3,4-b]indol-1-yl)ethanol

To a stirred solution of ethyl 2-(6-bromo-1′-ethyl-1,3,4,9-tetrahydro-8-isopropylpyrano[3,4-b]indol-1-yl)acetate (6.0 g, 0.015 mol) in THF (120 ml), was added 2.0 M solution of LiBH₄/THF (20 ml, 0.30 mol) via syringe during 30 min under a nitrogen atmosphere at room temperature. The mixture was refluxed for 10 hr, cooled, quenched with water and 5% HCl, and extracted with EtOAc. The organic phases were combined and washed with brine, dried over MgSO₄, and evaporated to give a residue, which was chromatographed on silica gel. Elution with hexane-EtOAc (7:3) gave the product (4.3 g, 80%). ¹H NMR (300 MHz, CDCl₃) δ 8.18 (b, NH), 0.92 (t, 3H), 1.35 (d, 6H), 1.98 (m, 3H), 2.19 (m, 1H), 2.54 (t, 1H), 2.75 (m, 2H), 3.17 (m, 1H), 3.71 (t, 1H), 4.03(m, 2H), 7.13 (dd, 1H), 7.45 (d, 1H), 7.96 (b, NH). ESI (+) MS m/e=367 (MH⁺), ESI (−) MS m/e=365 (MH⁻).

### COMPOUND 55: ETHYL 3-(1-ETHYL-1,3,4,9-TETRAHYDRO-1′-(2-HYDROXYETHYL)-8-ISOPROPYLPYRANO[3,4-B]INDOL-6-YL)PROPANOATE

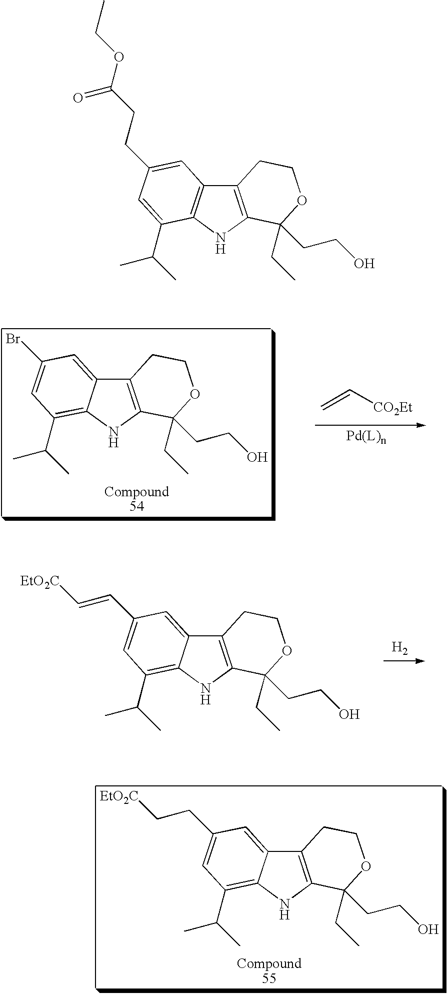

### 55.A. Synthesis of (E)-ethyl 3-(1-Ethyl-1,3,4,9-tetrahydro-1-(2-hydroxyethyl)-8-isopropylpyrano[3,4-b]indol-6-yl)acrylate

A suspension of Pd(OAc)₂ (0.2 g, 0.8 mmol), P(o-tolyl)₃ (0.25 g, 0.8 mmol), 2-(6-bromo-1-ethyl-1,3,4,9-tetrahydro-8-isopropylpyrano[3,4-b]indol-1-yl)ethanol (1.5 g, 4.1 mmol), triethylamine (1.5 ml, 11 mmol), and ethyl acrylate (1.8 ml, 16 mmol) in acetonitrile (45 ml) and stirred under a nitrogen atmosphere at 100° C. for 24 hr. The mixture was allowed to cool, quenched with water, worked-up with dichloromethane, and washed with brine. The organic layers were dried over MgSO₄ and concentrated under reduced pressure. The crude product was chromatographed on silica hexane/EtOAc (6:4) to give the product (0.9 g, 56%). %). ¹H NMR (300 MHz, CDCl₃) δ 8.07 (br, NH), 7.83 (d, 1H), 7.53 (d, 1H), 7.28 (d, 1H), 6.43 (d, 1H), 4.27 (m, 2H), 4.04 (m, 2H), 3.73 (m, 2H), 3.19 (m, 1H), 2.84 (m, 1), 2.77 (d, 1H), 2.52 (br, 1H), 2.20 (m, 1H), 2.09 (m, 1H), 1.92 (m, 1H), 1.38 (d, 6H), 1.35 (t, 3H), 0.95 (t, 3H); ESI (+) MS m/e=386 (MH⁺), ESI (−) MS m/e=384 (MH⁻).

### 55.B. Synthesis of Ethyl 3-(1-Ethyl-1,3,4,9-tetrahydro-1-(2-hydroxyethyl)-8-isopropylpyrano[3,4-b]indol-6-yl)propanoate

To a suspension of (E)-ethyl 3-(1-ethyl-1,3,4,9-tetrahydro-1-(2-hydroxyethyl)-8-isopropylpyrano[3,4-b]indol-6-yl)acrylate (0.8 g, 2.3 mmol) in 2% HCl in EtOH (80 ml) was added palladium on carbon (10%, 0.5 g). The mixture was stirred under an atmoshphere of hydrogen (1 am) at room temperature for 24 hr. The catalyst filtered through celite. The filtrate was evaporated at reduced pressure. The residue was neutralized with sat. NaHCO₃, extracted with EtOAc, and dried over MgSO₄. The solvent was concentrated under reduced pressure and purified by silica gel flash column chromatography hexane/EtOAc (6:4) to give the product (0.33 g, 38%). ¹H NMR (300 MHz, CDCl₃) δ 7.72 (br, NH), 7.18 (d, 1H), 6.91 (d, 1H), 4.15 (qrt, 2H), 4.02 (m, 2H), 3.70 (m, 2H), 3.17 (m, 1H), 3.04 (t, 2H), 2.83 (m, 1H), 2.68 (m, 3H), 2.18 (m, 1H), 2.05 (m, 1H), 1.96 (m, 2H), 1.36 (d, 6H), 1.26 (t, 3H), 0.94 (t, 3H), ESI (+) MS m/e=388 (MH⁺), ESI (−) MS m/e=386 (MH⁻).

### COMPOUND 56: 3-(1-ETHYL-1,3,4,9-TETRAHYDRO-1-(2-HYDROXYETHYL)-8-ISOPROPYLPYRANO[3,4-B]INDOL-6-YL)PROPANOIC ACID

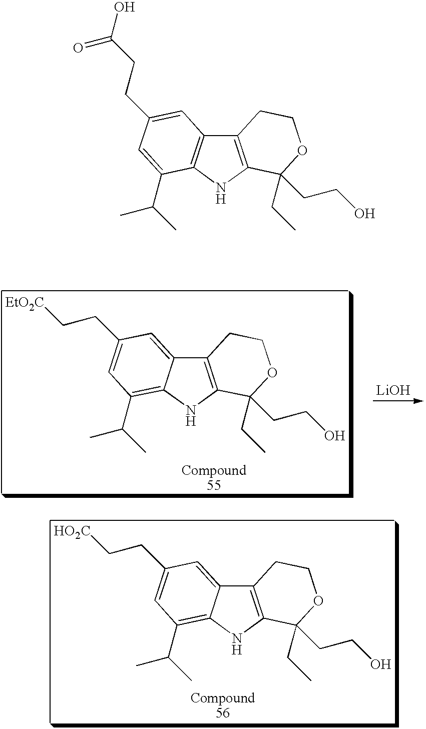

### 56.A. Synthesis of 3-(1-Ethyl-1,3,4,9-tetrahydro-1-(2-hydroxyethyl)-8-isopropylpyrano[3,4-b]indol-6-yl)propanoic Acid

To a stirred solution of ethyl 3-(1-ethyl-1,3,4,9-tetrahydro-1-(2-hydroxyethyl)-8-isopropylpyrano[3,4-b]indol-6-yl)propanoate (0.28 g, 0.72 mmol) in dioxane (6 ml) was added lithium hydroxide monohydrate (0.18 g, 4.3 mmol) and water (3 ml). The mixture was stirred at room temperature for 8 hr. It was concentrated under reduced pressure, neutralized with 5% HCl, extracted with EtOAc, and dried over MgSO₄. The solvent concentrated and purified by silica gel flash column chromatography dichloromethane/methanol (8:2) to give the product (0.09 g, 35%). ¹H NMR (300 MHz, CDCl₃) δ 7.77 (br, NH)), 7.19 (d, 1H), 6.91 (d, 1H), 4.04 (m, 2H), 3.68 (m, 2H), 3.16 (m, 1H), 3.06 (t, 2H), 2.85 (m, 1H), 2.74 (m, 3H), 2.18 (m, 1H), 1.98 (m, 3H), 1.35 (d, 6H), 0.94 (t, 3H); ESI (+) MS m/e=360 (MH⁺), ESI (−) MS m/e=358 (MH⁻).

### COMPOUND 57: 3-(1-ETHYL-1,3,4,9-TETRAHYDRO-1-(2-HYDROXYETHYL)-8-ISOPROPYLPYRANO[3,4-B]INDOL-6-YL)PROPAN-1-OL

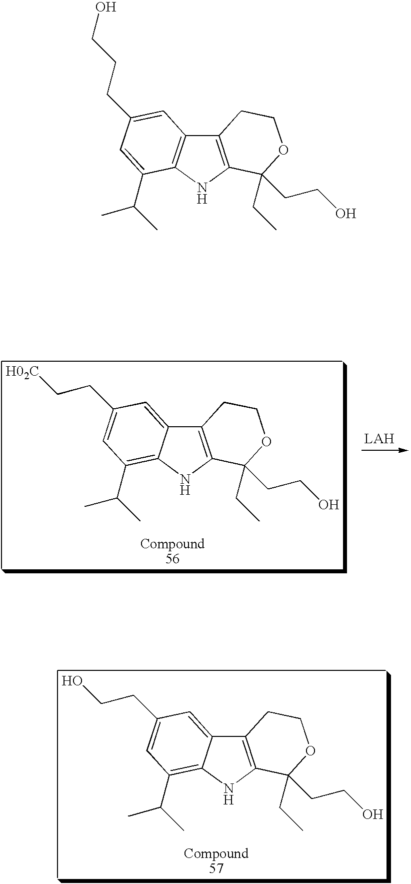

### 57.A. Synthesis of 3-(1-ethyl-1,3,4,9-tetrahydro-1-(2-hydroxyethyl)-8-isopropylpyrano[3,4-b]indol-6-yl)propan-1-ol

A solution of ethyl 3-(1-ethyl-1,3,4,9-tetrahydro-1-(2-hydroxyethyl)-8-isopropylpyrano[3,4-b]indol-6-yl)propanoate (0.18 g, 0.46 mmol) in anhydrous diethyl ether (15 ml) was stirred at room temperature under a nitrogen atmosphere. LiAlH4 (0.09 g, 2.4 mmol) was slowly added to the solution. The mixture was stirred for 18 hr, quenched with water and 5% HCl, extracted with EtOAc, dried over MgSO4, and concentrated under reduced pressure. The crude product was purified by silica gel flash column chromatography hexane/EtOAc (4:6) twice to give (31 mg) of the product (0.031 g, 20%). ¹H NMR (300 MHz, CDCl3) δ 7.70 (br, NH), 7.18 (d, 1H), 6.91 (d, 1H), 4.03 (m, 2H), 3.73 (m, 4H), 3.17 (m, 1H), 2.80 (m, 6H), 2.18 (m, 1H), 1.98 (m, 3H), 1.37 (d, 6H), 1.26 (br, 1H), 0.94 (t, 3H); ESI (+) MS m/e=346 (MH⁺), ESI (−) MS m/e=344 (MH⁻).

### Example 2

### Biological Data

Cox-1

Test compound and/or vehicle is incubated with human platelets (10⁸/ml) containing the phospholipase inhibitor MLnFP (100 μM) for 15 minutes at 37° C. Arachidonic acid (100 μM) is then added for a further 15 minute incubation period. The reaction is stopped by addition of 1 N HCl and neutralized with 1N NaOH. PGE₂ levels in the supernatant are determined using the Amersham EIA kit. Compounds are screened at 10 μM. Cox assays are described in Chan et al. 1999 J Pharmacol Exp Ther. 290:551-560; and Swinney et al. 1997, J Biol Chem. 272:12393-12398; both incorporated herein by reference.

Cox-2

Cyclooxygenase-2 (human recombinant, expressed in Sf9 cell, Cayman 60122) is used. Test compound and/or vehicle is pre-incubated with 0.11 U cyclooxygenase-2, 1 mM reduced glutathione (GSH), 500 μM phenol and 1 μM hematin for 15 minutes at 37° C. The reaction is initiated by addition of 0.3 μM arachidonic acid as substrate in Tris-HCl pH 7.7 and terminated after a 5 minute incubation at 37° C. by addition of 1N HCl. Following centrifugation, substrate conversion to PGE₂ is measured by an Amersham EIA kit. Compounds are screened at 10 μM. COX-2 assays are described in Riendeau, D., et al., 1997 Can. J. Physiol. Pharmacol. 75:1088-1095; and Warner, J. D., et al., 1999 Proc. Natl. Acad. Sci. U.S.A. 96: 7563-7568; both incorporated herein by reference.

Provided below in Table I are exemplary results for COX-1 and COX-2 inhibition by compounds described herein:

TABLE I

| Compound | COX-1 | COX-1 | COX-2 (IC50 | COX-2 |
|---|---|---|---|---|
| No. | (IC50 μM) | % Inhibition | μM) | % Inhibition |
| R-etodolac | >300 | −25 | >300 | −14 |
| 1 | <10 | 96 | <10 | 100 |
| 7 | | 94 | | 99 |
| 9 | | 88 | | 90 |
| 27 | >300 | 24 | >300 | 3 |
| 35 | >300 | −69 | >300 | −10 |
| 36 | >300 | −4 | >300 | 18 |
| 47 | 257 | 45 | 83.4 ± 20.7 | 78 |
| 52 | | 78 | | 97 |
| 53 | | 90 | | 96 |

Inhibition of β-Catenin

Inhibition of β-catenin was measured using a reporter assay based on the assay described in Korinek et al. 1997 Science 275:1784-1787 and employing the reporter plasmid TOPFLASH.

On Day 1, HEK-293 cells (ATCC) were plated in 24-well plates (VWR) at 40,000 cells per well in 450 μL DMEM+10×FBS media and incubated overnight at 37° C., 5% C0₂.

On Day 2, TOPFLASH plasmid (Upstate Cell Signaling Solutions, Va.), pGL3 control vector (Promega), and a plasmid encoding for constitutively expressed human β-catenin (Hans Cleversu) were separately diluted to 0.1 μg/μL in TE Buffer. Transfections were done using FuGene 6 Transfection Reagent (Roche). Transfection mixtures included either 8 μl of 0.1 μg/μl pGL3 in 400 μl serum free media (DMEM, Gibco) and 9.6 μl FuGene, or 8 μl of 0.1 μg/μl TOPFLASH and 16 μl of 0.1 β-catenin plasmid in 400 μl serum free media (DMEM, Gibco) and 9.6 μl FuGene. The transfection mixtures were gently mixed and incubated for 15-30 min at room temperature. Fifty μl of the appropriate transfection mixture was added dropwise to the 293 cells and the cells incubated overnight at 37° C., 5% CO₂.

On Day 3, the compounds to be tested were diluted to 0.25M in dimethylsulfoxide (DMSO). This solution was then used to make a 3× dilution of compound into DMEM+10% FBS, e.g., 100 μm to 300 μm. Two-hundred and fifty μl of the 3× diluted compound was added drop-wise to an appropriate well containing 500 μl of media. This was swirled gently. After mixing, 250 μl of the diluted 3× compound was added to another well and the procedure followed until the compound was diluted down three times. Plates were incubated for 24 hrs at 37° C., 5% CO₂. Experiments were performed in duplicate.

On Day 4, Luciferase activity was measured using a Promega Steady-Glo® luciferase assay system (Promega Cat. No. EC251) according to the manufactures instructions. The cells and Glo Lysis buffer were equilibrated to room temperature. Ten mls of Glo Lysis® Buffer was added to reconstitute the Steady-Glo® Assay Reagent. Five hundred μl of the Glo Lysis Buffer®/Assay Reagent were added to each well. The reaction was incubated for 5 min on a shaker at room temperature. 100 μl of lysate was transferred to a white 96-well plate and read on a Tecan (Research Triangle Park, N.C.) GENios microplate reader, using the luminescence setting.

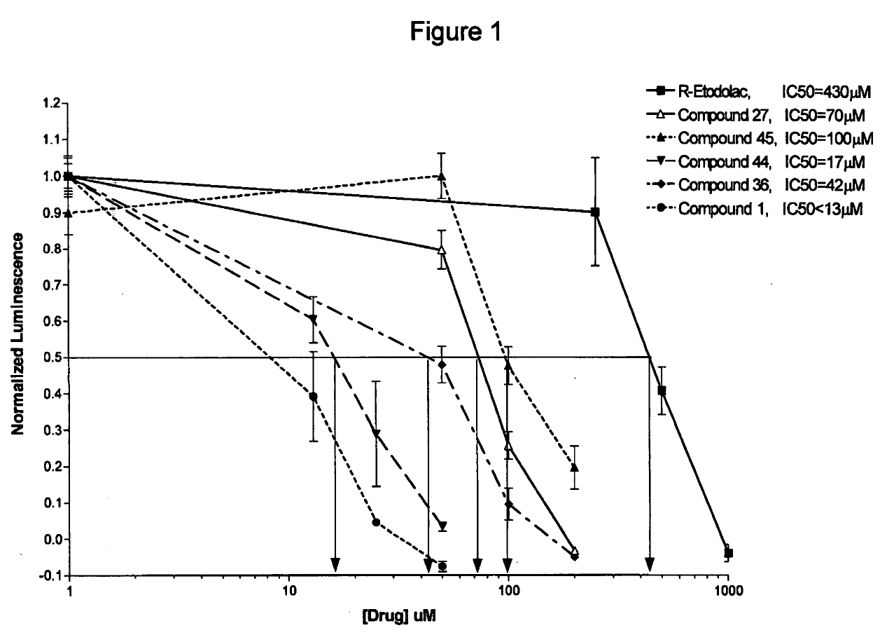

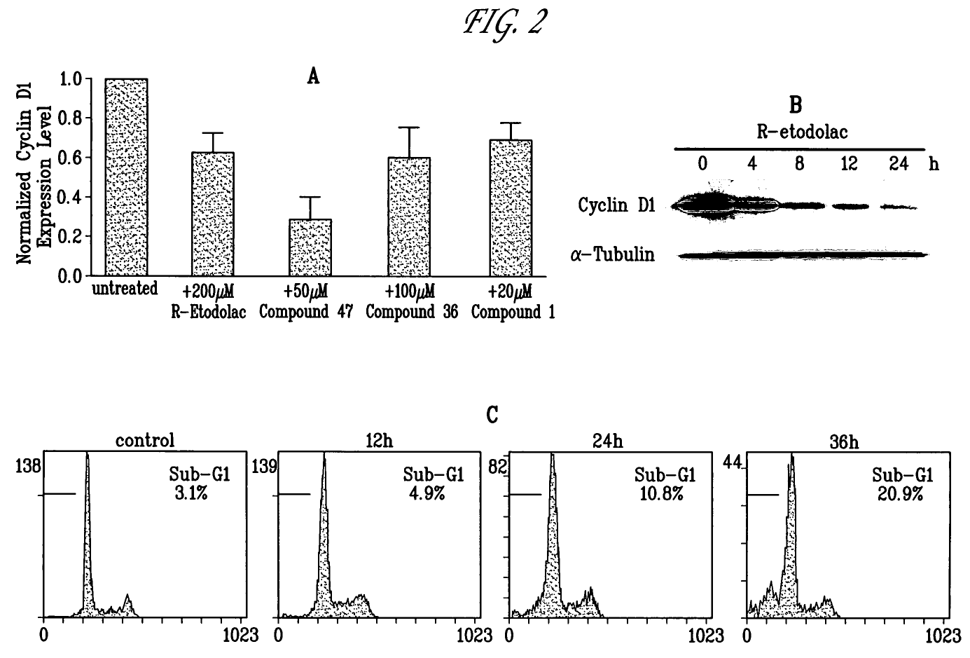

Inhibition of β-catenin:TOP flash by some compounds of the invention is shown in FIG. 1.

Cell Cytotoxicity

Normal prostate cells (PREC, Cambrex East Rutherford N.J.), prostate cancer cell line (LNCaP, ATCC), PBL (peripheral blood leukocytes-buffy coat San Diego Blood Bank), and primary CLL cells were incubated for one to two days in RPMI-1640 and 10% FBS (fetal bovine serum). They were plated in 96-well plates at 100,000 cells/well. Titrated concentrations of the compound to be tested were added to the culture medium. The cells were incubated three days at 37° C., 5% CO₂. Viability of the cells was assayed by standard MTT assay. Each drug concentration was done in duplicate.

MTT assay: 10 μl of 12 mM 3,[4,5-dimethylthiazol-2-yl]-2,5-diphenyltetrazolium bromide (MTT) Sigma) was added to each well. The cells were incubated at 37° C., 5% CO₂ for 4 hours. 100 μl of 20% SDS, 0.015M HCl was added to each well and the cells were incubated overnight. The plates were read at absorbance 595 nM.

Cytotoxicty results are shown in Table II and Table III.

TABLE II

| Compound | LNCap | PREC IC50 |
|---|---|---|
| No. | IC50 (nm) | (nm) |
| R-Etodolac | 122 | 416 |
| 1 | 14 | 53 |
| 7 | 12 | |
| 9 | 10 | |
| 22 | 163 | |
| 23 | 70 | |
| 24 | 80 | |
| 25 | 60 | |
| 26 | 95 | |
| 27 | 52 | 140 |
| 28 | 25 | |
| 29 | 80 | |
| 30 | 100 | |
| 31 | 37 | |
| 32 | 15 | |
| 33 | 132 | |
| 34 | 20 | |
| 35 | 68 | 220 |
| 36 | 30 | 160 |
| 37 | 60 | |
| 38 | 23 | |
| 39 | 46 | |
| 40 | 80 | |
| 41 | 51 | |
| 42 | 12 | |
| 43 | 77 | |
| 44 | 60 | |
| 45 | 8 | |
| 46 | 18 | |
| 47 | 9 | 14 |
| 48 | 60 | |
| 51 | 10 | |
| 52 | 9 | |
| 53 | 11 | |
| 54 | 7 | |
| 55 | 18 | |
| 56 | 235 | |
| 57 | 110 | |

TABLE III

| | CLL | | | | |
|---|---|---|---|---|---|
| Compound | IC50 | PBL | Compound | CLL | PBL |
| No. | (nm) | IC50 (nm) | No. | IC50 (nm) | IC50 (nm) |
| R-Etodolac | 200 | 350 | 35 | 100 | 140 |
| 1 | 72 | 76 | 39 | | 120 |
| 27 | 52 | 150 | 40 | 185 | |
| 31 | 244 | | 41 | 110 | |
| 32 | 250 | | 45 | | 90 |
| 33 | 98 | | 46 | | 20 |
| 36 | 50 | 160 | 47 | 21 | 60 |
| 38 | | 73 | 48 | 240 | |

Selected analogs were tested and compared in several tumor cell lines and their multidrug-resistant (MDR) sublines. The MDR cell lines used in these experiments have been extensively characterized in the literature and are resistant to several widely used anti-cancer drugs, such as doxorubicin, paclitaxel, etoposide, and others. As shown in Table IV, Table V and Table VI, the selected analogs were found to be about 10-20-fold more potent when compared to Etodolac. In addition, no appreciable loss of activity was observed in the multidrug resistant sub-lines, when compared to the parental cells.

TABLE IV

| | | Ovarian | | | | Leukemia |
|---|---|---|---|---|---|---|
| | Ovarian | MES- | Breast | Breast | Leukemia | HL- |
| | MES-SA | SA/Dx5 | MCF-7 | MCF-7 | HL-60 | 60/ADR |
| Compound No. | (parental) | (resistant) | (parental) | (parental) | (parental) | resistant |
| R-Etodolac | 700 | 430 | 625 | >1000 | 300 | 550 |
| 1 | 26 | 18 | 14 | 21 | 13 | 16 |
| 36 | 100 | 63 | 54 | 80 | 38 | 50 |
| 47 | 24 | 15 | 19 | 20 | 13 | 23 |

TABLE V

| | Kidney | | | Colon | Colon | | | |
|---|---|---|---|---|---|---|---|---|
| Compound | HEK- | Lung | Colon | HCT | HT- | Prostate | Prostate | Prostate |
| No. | 293 | A549 | SW480 | 116 | 29 | DU145 | PC3 | LNCap |
| R-Etodolac | 900 | 800 | 355 | 195 | 750 | 266 | 240 | 93 |
| 1 | 23 | 10 | 23 | 17 | 30 | 48 | 40 | 11 |
| 36 | NT | NT | NT | NT | 105 | NT | NT | NT |
| 47 | 47 | 26 | 16 | 8 | 25 | <20 | <20 | <20 |

TABLE VI

| Compound | RMPI8226 |
|---|---|
| No. | micromolar |
| R-Etodolac | 140 |
| 36 | 75 |
| 47 | 16 |
| 54 | 20 |
| 55 | 37 |
| 56 | 238 |
| 57 | 320 |

Antiangiogenic Assay

To determine the effects of COX inhibitors on angiogenesis in vivo, selective compounds will be tested in the mouse and rat corneal micropocket assay. The mouse corneal neovascularization micropocket model is performed with materials, reagents and procedures essentially as described by Muthukkauppah et al., 1982 J. Natl. Cancer Inst., 69, 699-708. In this assay, a pellet containing basic fibroblast growth factor (FGF) is implanted into the corneal stroma of the mouse and the newly formed vessels are measured using a slit lamp. In this model, COX-2 is expressed in the endothelial cells of the newly developed blood vessels. The ability of a compound of the invention to inhibit FGF-induced angiogenesis in the mouse will be tested using the above method. The inhibitory effects of the compounds of the invention in the mouse cornea model will be tested using another angiogenic stimulus, vascular endotherlial growth factor (VEGF).

Cyclin D1

Cyclin D1 Transcript Expression Levels as measured by quantitative PCR assay. LNCaP cells were cultured at 37° C., 5% CO₂ for 24 hours untreated or in the presence of R-etodolac (200 μM), compound 42 (50 μM), compound 36 (100 μM), or compound 1 (20 μM) (see Table II for structures). Cells were harvested by trypsinization, washed with PBS, and stored at −80° C. Total cellular RNA was prepared from cell pellets using the RNEasy® Mini kit (Qiagen, Inc., Valencia, Calif.). RNA was quantified by spectrophotometer. Approximately 21 g of RNA was used to prepare cDNA using the ThermoScript™ RT-PCR System (Invitrogen, Carlsbad, Calif.).

The levels of cyclin D1 transcripts in the cDNA samples were measured using a quantitative PCR (qPCR) assay specific for cyclin D1. The cyclin D1 transcript was amplified using the following primer pair:

| | |
|---|---|
| Cyclin D1 for: | |
| 5′- AATGACCCCGACCGATT-3′ | (SEQ ID NO:1) |
| Cyclin D1 rev: | |
| 5′- GCACAAGAGGCAACGAAG G-3′ | (SEQ ID NO:2) |

The cyclin D1 primers are described in a manuscript from Takayasu et al. (2001 Clin. Cancer Res. 7:901-908). All assays were performed in duplicate. All qPCR assays were performed and analyzed using a Bio-Rad iCycler (Bio-Rad, Hercules, Calif.). The levels of cyclin D1 transcripts were normalized for total input cDNA by performing a separate assay to detect the levels of a housekeeping gene (18 s) using the following primer pair:

| | | |
|---|---|---|
| 18s for: | 5′-CGCCGCTAGAGGTGAAATTC-3′ | (SEQ ID NO:3) |
| 18s rev: | 5′-TTGGCAAATGCTTTCGCTC-3′ | (SEQ ID NO:4) |

The samples were normalized for 18 s transcript levels using the method of Livak et al. (2001 Methods 25:402-408). The level of cyclin D1 transcripts in the control sample was set to 1. FIG. 2 represents the averaged normalized cyclin D1 transcripts±standard deviation for three independent experiments (two independent experiments for compound 1). The data show that compound 42, compound 36, and compound 1 inhibit cyclin D1 mRNA expression.

Western blot analysis of LNCaP cell lysates from cells treated with R-etodolac, compound 42, compound 36, or compound 1 using a monoclonal antibody specific for Cyclin D1 (BD Pharmingen) confirmed that the compounds reduced Cyclin D1 protein expression.

R-etodolac also down regulates cyclin D1 in MM cells. In U266 cells, which are less sensitive to R-etodolac compared to other MM cells, 1 mM R-etodolac significantly downregulates cyclin D1 at 4 hours, as determined by immumoblotting (as described below) with anti cyclin D1 antibodies (Cell Signaling, Beverly, Mass.) (FIG. 2B). This inhibition of cyclin D 1 in MM cells occurs without significant change of cell cycle profile (Sub-G1 cells gradually increase to 20.9% of total at 36 hours (FIG. 2C) (cell cycle analysis was done as described below). These data suggest that R-etodolac downregulates expression of cyclin D1 in MM cells and delays progression through the cell cycle.

Other Cyclin D proteins have been shown to be dependent on the Wnt/beta-catenin pathway (e.g., cyclin D2—Briata et al. 2003 Mol. Cell 12:1201-11) and would be expected to be affected by the compounds of the invention in a similar way as Cyclin D1. The inhibition of cyclin D expression by the compounds of the invention can also be used as a biomarker of the efficacy of these compounds.

### Example 3

### Daudi Lymphoma Murine Xenograft Model Mice Studies

Materials

Male SCID mice, 6-8 weeks of age, obtained from Simonsen Laboratories, Inc. (Gilroy, Calif.) were housed in groups of five.

The Daudi human Burkitt Lymphoma cells were obtained from American Type Culture Collection and were inoculated subcutaneously (1.0×10⁷ cells/mouse) on the flanks of SCID mice. After the tumors reached approximately 100 mm³ treatment was initiated.

Body weights and tumor volume of all mice were measured and recorded twice weekly. Tumors were measured in three dimensions and volume calculated using the formula 4/3πr3. Time for the tumors (days) to grow to 4× and 8× the initial volume at dosing were assessed. Study compounds were administered at 125 or 250 mg/kg/day (M-F) via oral gavage until the end of the study.

Efficacy

The efficacy of chlorambucil (2 and 3 mg/kg/d), (R-etodolac) (400 mg/kg/d) and compound 47 (250 mg/kg/d), compound 26 (250 mg/kg/d), and compound 1 (125 mg/kg/d) against Daudi derived tumors in male SCID mice were studied. R-etodolac and compounds 1, 26 and 47 were prepared in sesame oil. Both chlorambucil (ip, 0.1 ml) and compounds of the invention (per os [p.o.], 0.31 ml) were dosed daily (Monday to Friday) for two weeks. SDX-101 (0.31 ml) was dosed p.o. daily until the end of the study. Slight body weight loss (<3%) was observed at the beginning of the study in chlorambucil (2 mg/kg/d), compounds 47 and 36 treated groups. However, all treated mice recovered after Day 2 and maintained their weights. There was no body weight loss observed in other treatment groups. At termination of the study, the control group mean tumor volume was 1583 mm³. The mean tumor volume of chlorambucil treated groups were 864 and 766 mm³ with 2 and 3 mg/kg/d chlorambucil treatment, respectively. The mean tumor volume of R-etodolac and compounds 1, 36 and 47 were 802, 996, 1011, and 1157 mm³ with compounds 47, 36, 1 and R-etodolac treatment, respectively. Analysis of variance (ANOVA) of tumor volume of control and chlorambucil groups on Day 20 showed a p-value of 0.001 and 0.0003 between the control group vs. 2 and 3 mg/kg/d chlorambucil treated groups, respectively. ANOVA also showed a p-value of 0.007 and 0.03 between the control group vs. compound 47 and compound groups, respectively. At termination of the study, tumor samples along with liver, kidney, and spleen samples from each group were collected and fixed in 10% buffered formalin for histopathology. Histological analysis of all liver, spleen and kidney tissues indicated that all tissues appeared normal.

Table VII shows the time for the tumors to grow to 4× and 8× the initial volume when mice were administered chlorambucil, R-etodolac and compounds 1, 36 and 47. These data indicate that the compounds of the invention inhibit tumor growth in the Daudi mouse model.

TABLE VII

| Group | 4X Growth (d) | 8X Growth (d) |
|---|---|---|
| Control | 8.9 | 14.5 |
| Chlorambucil | 16 | 21 |
| R-etodolac | 11 | 17 |
| Compound 47 | 16 | 21.5 |
| Compound 1 | 15.8 | 21.1 |
| Compound 36 | 13.5 | 20.8 |

### Example 4

### Antitumor Activity of R-Etodolac (SDX-101) in Resistant Multiple Myeloma

Materials and Methods

R-etodolac was dissolved in DMSO (250 mM) and stored at −20° C. until use. IL-6 and IGF-1 were purchased from R&D Systems (Minneapolis, Minn.). Pan caspase inhibitor Z-VAD-FMK (Calbiochem, San Diego, Calif.) was dissolved in DMSO, stored at −20° C., and used at 25 μM.

MM-derived cell lines: dexamethasone sensitive (MM.1S) and dexamethasone-resistant (MM.1R) human MM cell lines were obtained from Dr. Steven Rosen (Northwestern University, Chicago, Ill.) and are described in Moalli et al. Blood 79:213-222 (1992). Doxorubicin resistant (Dox-40) and melphalan resistant (LR5) RPM18226 human MM cell lines were obtained from Dr. William Dalton (Moffitt Cancer Center, Tampa, Fla.) (Damiano-J. S. et al., Blood 93:1658-1667 (1999). U266 MM cells were obtained from the American Type Culture Collection (Manassas, Va.). Human SUDHL4 (DHL4) lymphoma cells were provided by Dr. Margaret Shipp (Dana-Farber Cancer Institute, Boston, Mass.). Patient MM cells (96% CD38 positive/CD45RA negative) were purified from patient BM samples, as described previously (Tai et al., J. Immunol. Methods 235:11-9 (2000))

All of the MM cell lines were cultured in RPMI 1640 containing 10% fetal bovine serum (Sigma Chemical Co., St. Louis, Mo.), 2×10−3 M L-glutamine, 100 units/ml penicillin (Pen), and 100 μg/ml streptomycin (GIBCO., Grand Island, N.Y.). Drug-resistant cell lines were cultured with doxorubicin or dexamethasone (Dex) to confirm their lack of drug sensitivity.

BMSC Cultures

BM specimens were obtained from patients with MM. Mononuclear cells separated by Ficoll-Hipaque density sedimentation were used to establish long-term BM cultures, as described previously (Hideshima T. et al., Blood 96:2943-2950 (2000)). When an adherent cell monolayer had developed, cells were harvested in HBSS containing 0.25% trypsin and 0.02% EDTA and were washed and collected by centrifugation.

DNA Synthesis

Proliferation was measured as described in Hideshima T. et al., Blood 96:2943-2950 (2000). MM cells (3×10⁴ cells/well) were incubated in 96-well culture plates (Costar, Cambridge, Mass.) in the presence or absence of media, SDX-101 (R-etodolac), and/or Dex, melphalan or recombinant IL-6 (Genetics Institute, Cambridge, Mass.) for 24 h at 37° C. DNA synthesis was measured by [³H]thymidine (Perkin Elmer, Boston Mass.) uptake. Cells were pulsed with [³H]thymidine (0.5 μCi/well) during the last 8 h of 24-h cultures. All of the experiments were performed in triplicate.

Growth Inhibition Assay

The inhibitory effect of SDX-101 on MM and BMSC growth was assessed by measuring 3-(4,5-dimethylthiazol-2-yl)-2,5-diphenyl tetrasodium bromide (MTT, Chemicon International Inc. Temecula, Calif.) dye absorbance of the cells. Cells from 48-h cultures were pulsed with 10 μl of 5 mg/ml MTT to each well for the last 4 h of 48-h cultures, followed by 100 μl of isopropanol containing 0.04 N HCl. Absorbance was measured at 570 nm using a spectrophotometer (Molecular Devices Corp., Sunnyvale Calif.).

Cell Cycle Analysis

MM.1S cells or U266 cells were treated with varying concentrations of SDX-101 or control media were harvested, washed with PBS, fixed with 70% ethanol, and treated with 10 μg/ml RNase (Roche Diagnostics Corp., Indianapolis, Ind.). Cells were then stained with PI (Sigma; 5 μg/ml), and cell cycle profile was determined using the program M software on an Epics flow cytometer (Coulter Immunology, Hialeah, Fla.), as in prior studies (Hideshima T. et al., Blood 96:2943-2950 (2000))

Immunoblotting

MM.1S cells were cultured with SDX-101, in the presence or absence of capase inhibitors, and harvested, washed, and lysed using lysis buffer [50×10⁻³ M Tris-HCl (pH 7.4), 150×10⁻³ M NaCl, 1% NP40, 10 mM sodium pyrophosphate, 5×10⁻³ M EDTA, 1 mM EGTA, 5×10⁻³ M NaF, 2×10⁻³ M Na₃VO₄, 1×10⁻³ M PMSF, 5 μg/ml leupeptine, and 5 μg/ml aprotinin]. For detection of Cyclin D1, p21, Bcl-2, Bax, Caspase-3, p53, PARP, Caspase-8, and Caspase 9, cell lysates were subjected to SDS-PAGE, transferred to PVDF membrane (Bio-Rad Laboratories, Hercules, Calif.), and immunoblotted with anti-ADP-ribose polymerase (PARP), anti-Caspase-8, anti-Caspase-9, anti-p21, anti-Bcl-2, anti-Bax, anti-p53, anti-caspase-3 (Santa Cruz Biotechnology, Santa Cruz, Calif.).

Effect of SDX-101 on Paracrine MM Cell Growth in the Bone Marrow (BM)

To evaluate the effect of SDX-101 on growth of MM cells adherent to bone marrow stromal cells (BMSCs) MM.1S cells (3×10⁴ cells/well) were cultured for 24 hours in BMSC-coated 96-well plates (Costar, Cambridge, Mass.), in the presence or absence of SDX-101. DNA synthesis was measured as described herein.

Statistical Analysis

Statistical significance of differences observed in R-etodolac treated compared with control cultures was determined using a Student t test. The minimal level of significance was p<0.01.

Isobologram

The interaction between R-etodolac and Dex was analyzed using CalcuSyn software program (Biosoft, Ferguson, Mo.) to determine whether the combination was additive or synergistic, as described previously (Raje N, et al. Blood. 2004;104:4188-4193; Chou T C, Talalay P. Adv Enzyme Regul. 1984;22:27-55. When the Combined Index (CI)≧1, this equation represents the conservation isobologram and indicates additive effects. A CI<1.0 indicates synergism.

Results

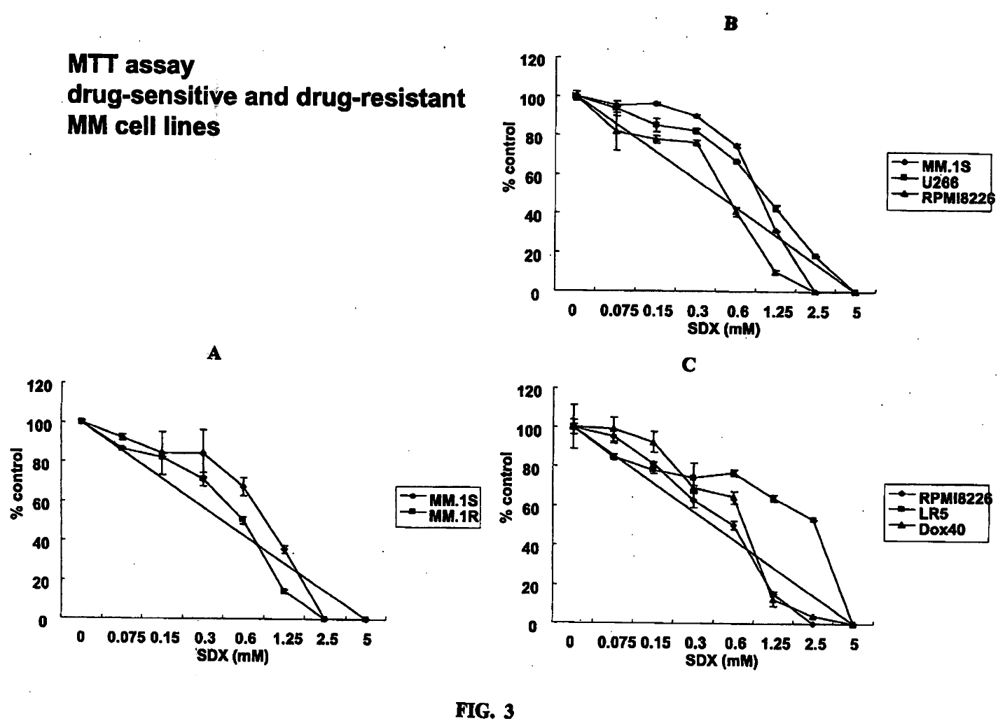

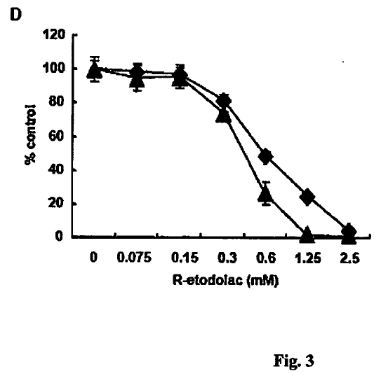

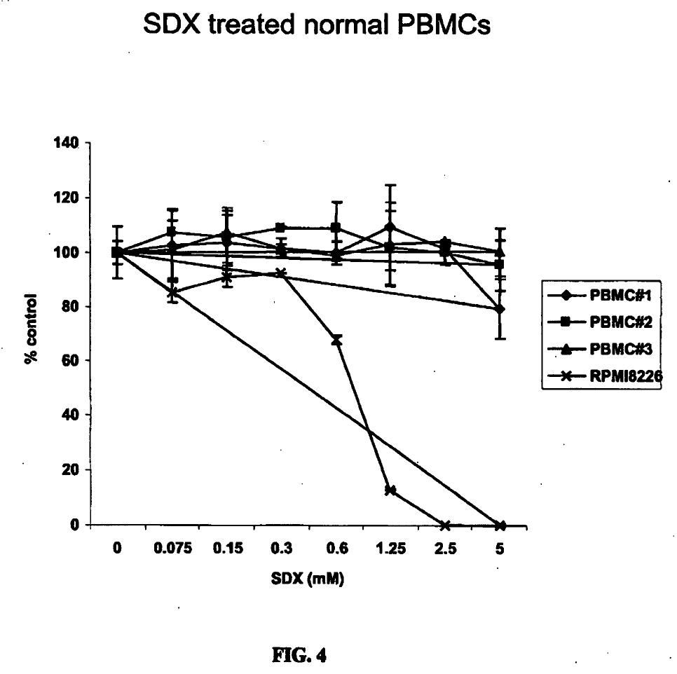

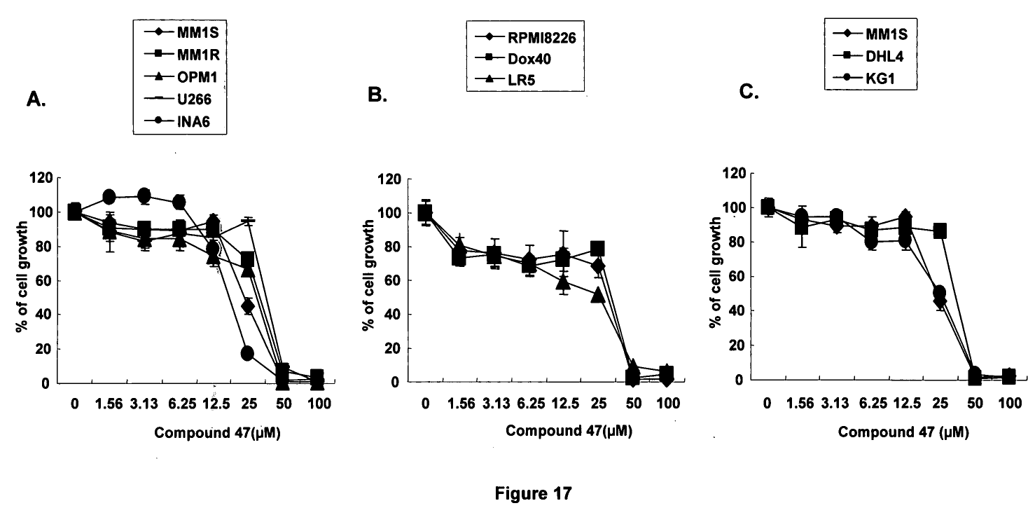

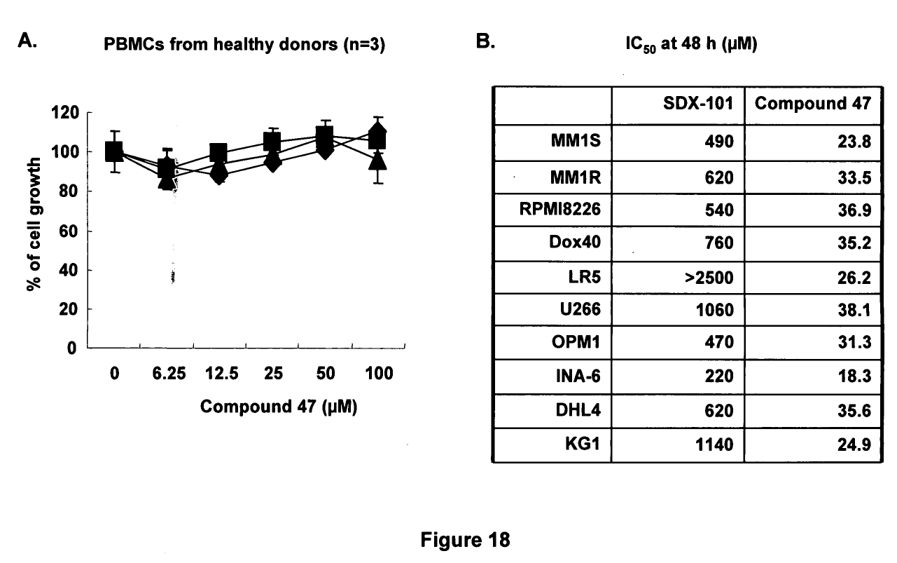

A. As shown FIG. 3, the growth of various MM cell lines was completely inhibited by SDX-101 (2.5 mM for Dox40, RPM18226, and 5 mM for LR5). Fifty % growth inhibition (IC50) in U266 was noted at a concentration of about 1.25 mM. IC50 of SDX-101 in RPM18226, Dox40, and LR5 was about 0.6, 0.8, and 2.5 mM, respectively. Dex-sensitive (MM.1S) and -resistant (MM.1R) MM cell lines were similarly examined. As can be seen in FIG. 3A, growth of both cell lines was completely inhibited by SDX-101 (2.5 mM). IC50 of SDX-101 in MM.1S and MM.1R cells was 1.0 and 0.6 mM, respectively. SDX-101 was also tested against bortezomib-resistant DHL4 cells (FIG. 3D). These data demonstrate that SDX-101 effectively inhibits the growth of chemoresistant MM cells at pharmacologically achievable doses and can overcome resistance to doxorubicin, melphalan, Dex and bortezomib. Peripheral blood mononuclear cells (PBMC)s from three normal volunteers were also examined for their susceptibility to SDX-101. As can be seen in FIG. 4, SDX-101 triggered only 3-17% cytotoxicity of PMBCs from three normal volunteers. These data demonstrate that SDX-101 induces cytotoxicity in MM cells but not normal PMBCs. Similar results were seen with compound 47 except that the compound was 10-fold more active than SDX-101 (FIGS. 17 and 18).

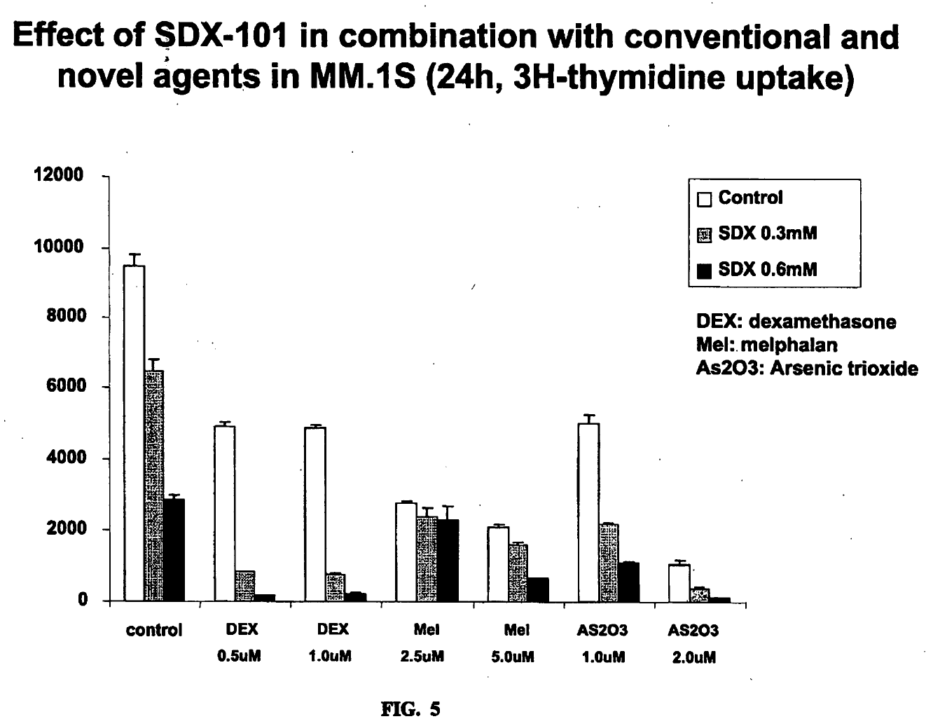

To determine whether SDX-101 enhances cytotoxicity of conventional therapies or novel agents, the effect of Dex, melphalan, and arsenic trioxide together with SDX-101 on proliferation of Dex-sensitive MM.1S cells was tested. As can be seen in FIG. 5, ³H-thymidine uptake assays at 24 hours revealed that SDX-101 alone (0.3 mM and 0.6 mM), Dex alone (0.5 μM and 1.0 μM), melphalan alone (2.5 μM and 5.0 μM) and arsenic trioxide alone (1.0 μM and 2.0 μM) each significantly inhibited MM.1S cell growth in a dose-dependent fashion and, furthermore, showed that their growth inhibitory effects were either synergistic with respect to SDX-101 with Dex or arsenic trioxide and additive with respect to SDX-101 with melphalan.

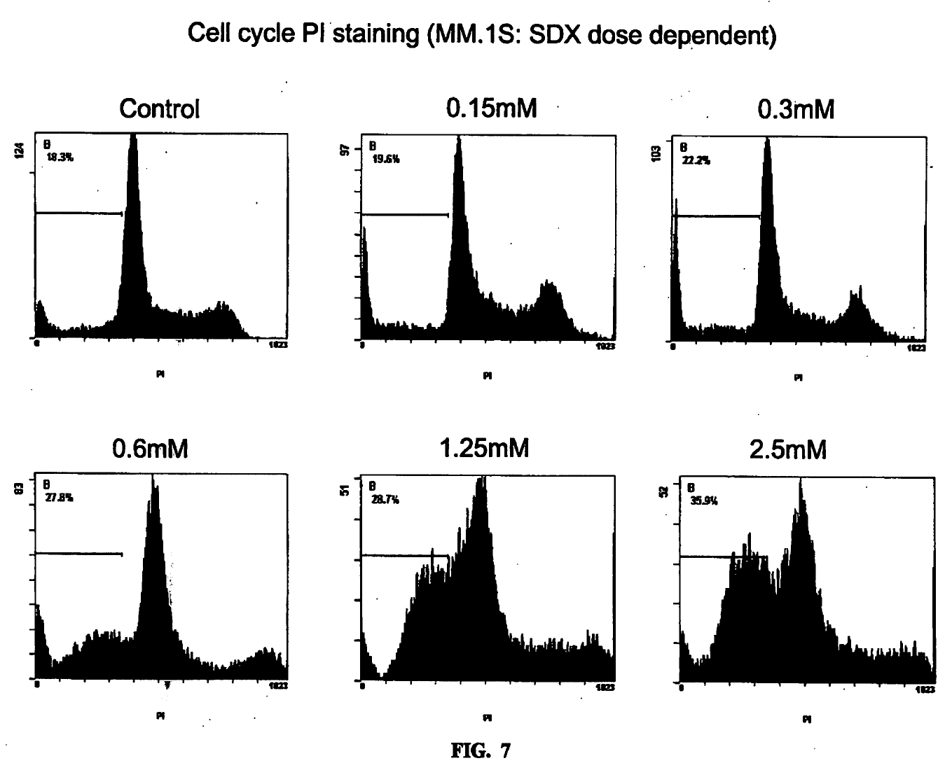

To further analyze the mechanism of SDX-101-induced inhibition of DNA synthesis and to determine whether SDX-101 induced apoptosis of MM cells, the cell cycle profile of MM.1S cells cultured with media or various concentrations of SDX-101 was examined. After incubation, cells were harvested and stained with propidium iodide (PI). As shown in FIG. 7, SDX-101 induced a progressive increase in sub-G0/G1 phase cells.

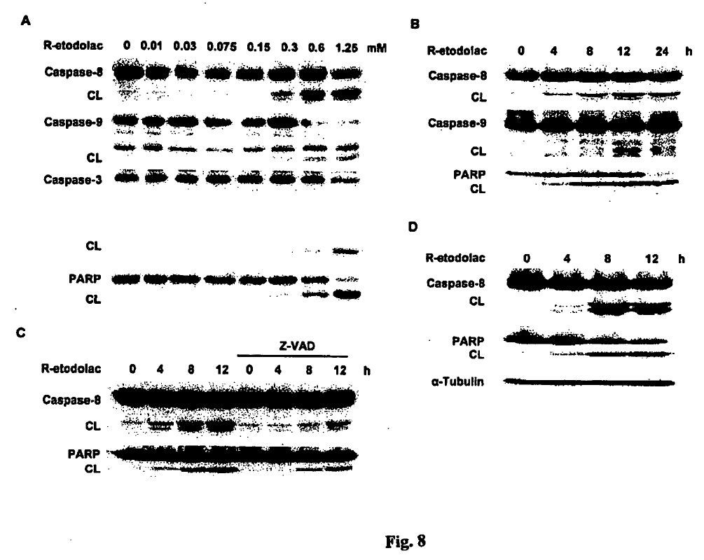

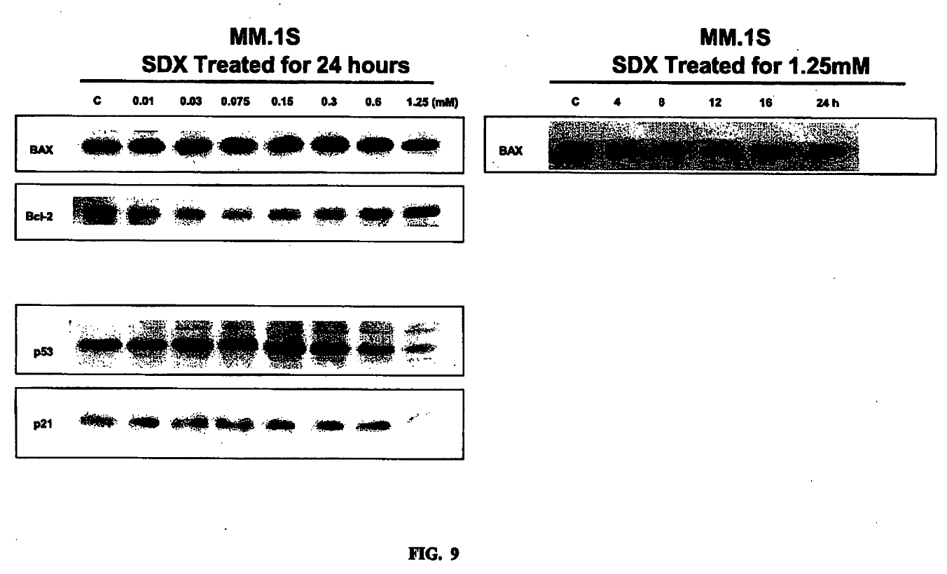

Apoptosis triggered by SDX-101 was further confirmed by cleavage of PARP, Caspase-8, and Caspase-3 in MM.1S cells (FIG. 8). Apoptosis occurred despite up-regulation of p21 (FIG. 9). No changes in Bcl-2 or Bax expression were induced by SDX-101 (FIG. 9). Conversely, the pan-caspase inhibitor Z-VAD-FMK blocks R-etodolac-induced caspase-8 and PARP cleavage in MM.1S cells (FIG. 8C). As in MM.1S cells, R-etodolac also induces caspase activation and PARP cleavage in RPMI8226 cells (FIG. 8D). These results indicate that cytotoxicity triggered by R-etodolac, like bortezomib, As₂O₃ and 2ME-2 is mediated via caspase-8/-9/-3 activation and apoptosis.

B. Subtoxic Doses of R-Etodolac Induce Upregulation of Mcl-1s and Dex-Induced Apoptosis in MM Cells

Since Mcl-1 plays an important role in proliferation and anti-apoptosis as well as drug resistance, levels of Mcl-1 protein expression were assessed. Immunoblotting showed that subtoxic doses of R-etodolac (0.15 or 0.3 mM) induced upregulation of Mcl-1S, which is a short splicing variant of the mcl-1 gene with antagonistic potential against Mcl-1L. Although 24 hour treatment with a toxic dose of R-etodolac (0.6 mM) doesn't significantly change expression of Bcl-2 family proteins such as Mcl-1L, Bcl-xL, Bax, and Bcl-2, it does cleave Mcl-1L, with loss of its anti-apoptotic activity (FIG. 11A). Cleavage of Mcl-1L triggered by toxic doses of R-etodolac (0.6 mM) are detected as early as 8 hours (FIG. 11B). These results suggest that upregulation of Mcl-1S and cleavage of Mcl-1L are associated with the apoptotic effect of R-etodolac.

To delineate mechanisms underlying the synergistic anti-MM activity of combined R-etodolac and Dex, Mcl-1 expression and activation of apoptotic signaling in MM.1S cells cultured with either media or 1.0 μM Dex, in the presence of subtoxic dose of R-etodolac (0.15 or 0.3 mM) was assessed. Immunoblotting shows that subtoxic doses of R-etodolac induced upregulation of Mcl-1S and significantly enhanced Dex-induced cleavage of Mcl-1L, caspase-8/-9, and PARP (FIG. 11C). These results indicate that Mcl-1S induced by R-etodolac triggers Dex-mediated apoptosis. Compound 47 also induced apoptosis via caspase activation and PARP cleavage.

C. R-Etodolac Induces Apoptosis in Patient MM Cells

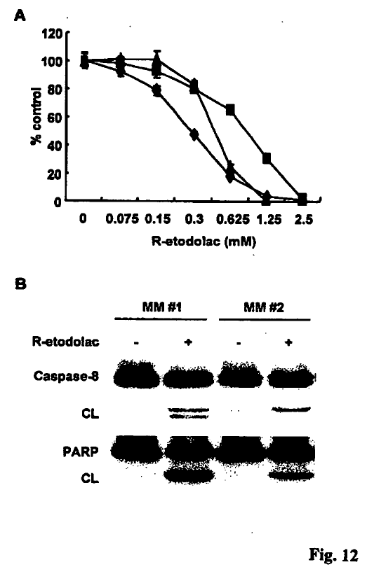

R-etodolac induces dose-dependent cytotoxicity in CD138 positive BM cells isolated from 2 patients with MM who were refractory to multiple prior therapies including dexamethasone, melphalan, thalidomide, or bortezomib, with IC50 of 0.30 and 0.89 mM, respectively (FIG. 12A). It also induced apoptosis, evidenced by caspase-8 activation and PARP cleavage on immunoblotting (FIG. 12B). These results demonstrate that R-etodolac also has anti-MM activity against refractory patient MM cells.

D. SDX-101 Overcomes IL-6 and IGF-1 Mediated Growth and Anti-Apoptosis in MM Cells.

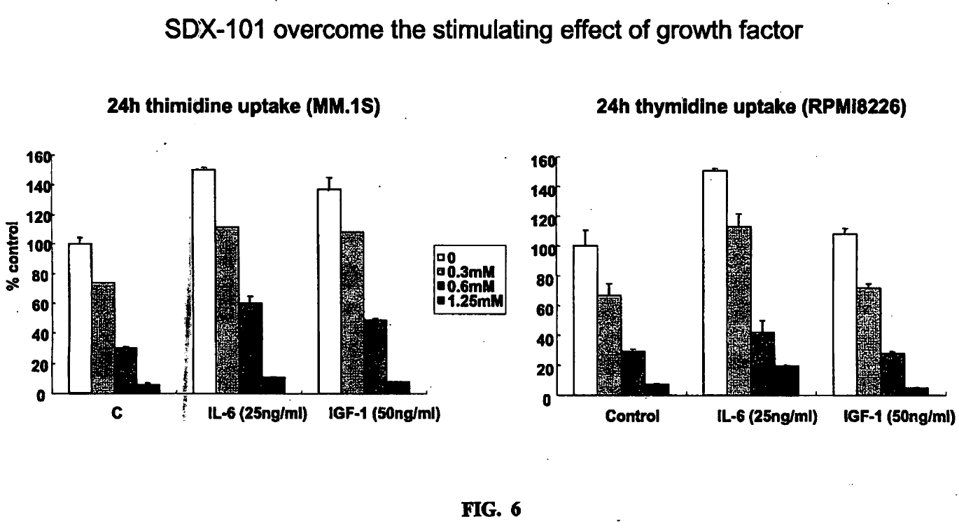

IL-6 and IGF-1 mediate both growth and anti-apoptosis in MM cells. Experiemtns were performed to determine if SDX-101 could overcome the effects of exogenous IL-6 and IGF-1. Although IL-6 (25 ng/ml) and IGF-1 (50 ng/ml) triggered an increase in thymidine uptake at 24 hours in MM.1s and RPM18226 cells when compared to MM.1S and RPMI8226 cell growth in cultures with media alone, SDX-101 inhibited this response in a dose-dependent fashion (FIG. 6) Thus neither IL-6 nor IGF-1 protected against R-etodolac induced anti-MM activity.

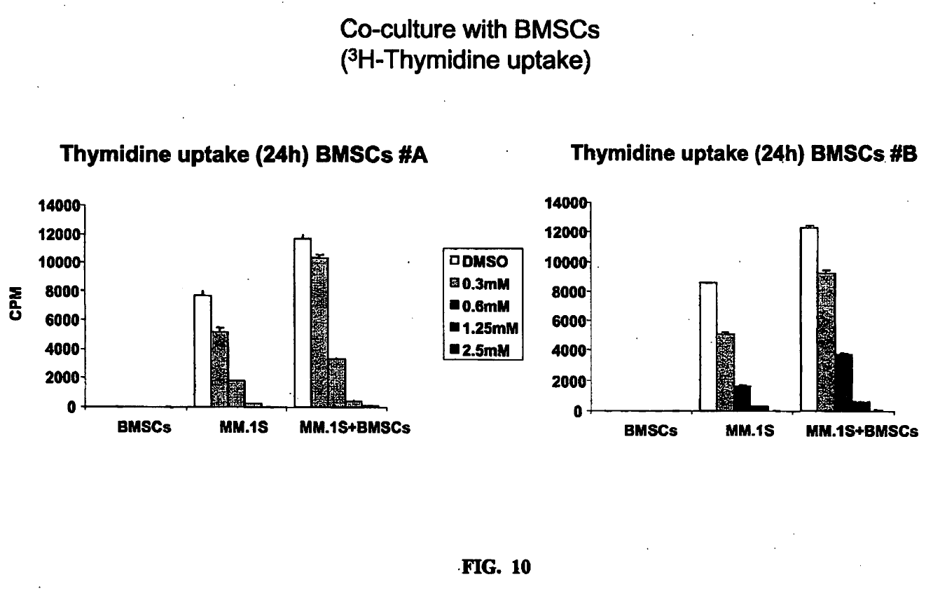

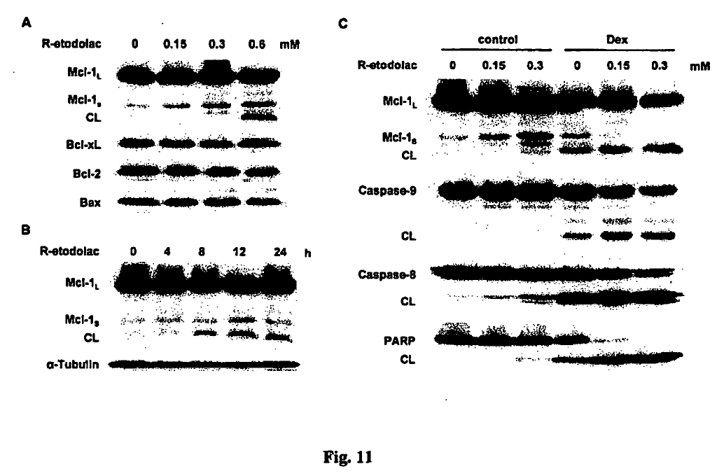

The effect of SDX-101 on paracrine MM cell growth in the BM milieu was investigated. MM.1s cells cultured with or without BMSCs in the presence or absence of SDX-101. Tumor cell adherence to BMSCs triggered increased [³H]thymidine uptake of MM.1S cells and SDX-101 inhibited this up-regulation of growth in a dose-dependent manner (FIG. 10). Because adherence of MM cells to BMSCs triggers increased secretion of IL-6 and IGF-1 in culture supernatants, these results are consistent with the observed inability of exogenous IL-6 or IGF-1 to overcome the growth-inhibitory effects of SDX-101 (FIG. 6). These data suggest that SDX-101 overcomes the protective effect of the BM microenvironment against conventional chemotherapy. Compound 47 was also able to overcome the protective effects of IL-6, IGF-1, and BMSCs.

### Example 5

### R-Etodolac Ehnhances Dex-Induced Cytotoxicity even in Dex-Resistant OPM1 Cells

Materials:

OPM1 myeloma cell line were obtained from Dr. Edward Thompson (University of Texas Medical Branch, Galveston, Tex.) and cultured in RPMI-1640 containing 10% fetal bovine serum (FBS, Sigma Chemical Co., St. Louis, Mo., USA), 2 μM L-glutamine, 100 U/ml penicillin, and 100 μg/ml streptomycin (GIBCO, Grand Island, N.Y., USA). Anti-IκBα antibody was purchased from Santa Cruz Biotech (Santa Cruz, Calif.).

Results:

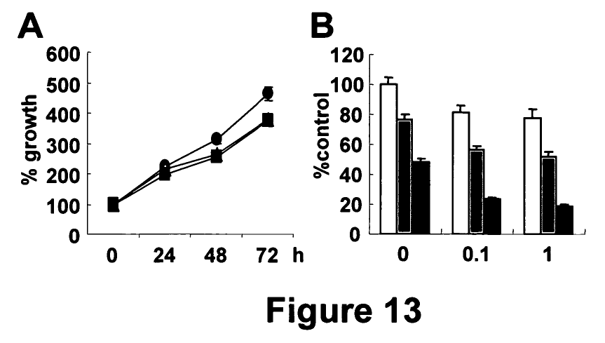

The growth inhibitory effect of 0.1 or 1 μM Dex in OPM1 cell line at 24-48-72 h was investigated by MTT assay, as described herein. As shown in FIG. 13A, Dex does not inhibit MM cell growth. Also investigated was whether R-etodolac enhances the growth inhibitory effect of Dex even in Dex-resistant OPM1 cells. OPM1 cells were cultured with 0.1 or 1.0 μM Dex for 24 h before addition of 0.3 or 0.6 mM R-etodolac for 24 h. As shown in FIG. 13B, R-etodolac enhances growth inhibition mediated by Dex, as analyzed by MTT assay. While 1.0 μM Dex or 0.6 mM R-etodolac alone triggered 21.9% or 51.5% cytotoxicity in MTT assays, respectively, combined R-etodolac and Dex at these concentrations induced 81.7% cytotoxicity and triggered synergistic (SQ 1.11) anti-proliferative effects. These results demonstrate that R-etodolac augments Dex-mediated cytotoxicity in Dex-resistant OPM1 cells at plasma concentrations which have been clinically achieved in a clinical trial of CLL patients (0.3-0.6 mM).

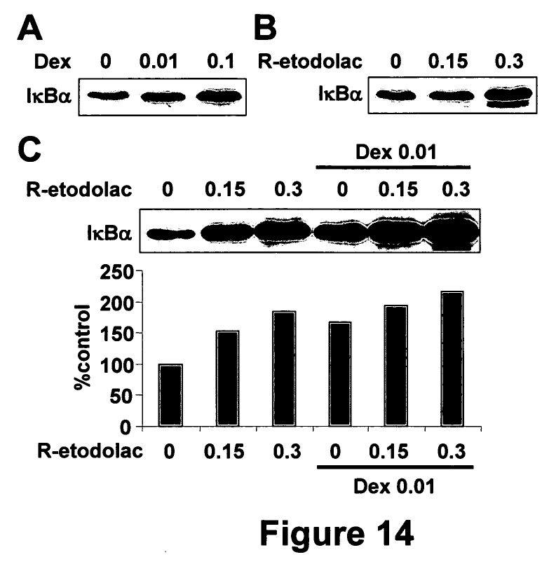

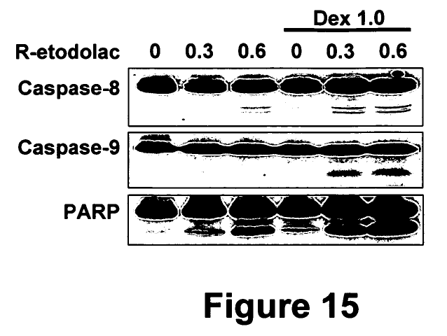

Immunoblot analysis (as described herein) was used to delineate the mechanism underlying the enhancement of combined R-etodolac with Dex-induced cytotoxicity in OPM1 cells. It has been previously demonstrated that nuclear factor-kappa B (NFκB) activity mediates survival and resistance to conventional chemotherapeutic drugs in MM cells. Since Dex triggers an increase in IκBα protein levels and thereby inhibits NFκB activity, it was determined whether R-etodolac and Dex upregulate IκBα expression levels in OPM1 cells. As shown in FIGS. 14A and 14B, Dex (0.01 or 0.1 μM) and R-etodolac (0.15 or 0.3 mM) upregulate IκBα at 24 h; moreover, the combination of both drugs further enhanced this upregulation (FIG. 14C). Also determined, by immunoblot analysis, was the apoptotic signaling in OPM1 cells cultured with either media alone or 1.0 μM Dex, in the presence of 0.3 or 0.6 mM R-etodolac. As shown in FIG. 15, R-etodolac enhanced Dex-induced cleavage of caspase-8/-9, and PARP, hallmarks of apoptosis. Taken together, these data suggest that R-etodolac upregulates the expression of IκBα and induces apoptosis via caspases activation and PARP cleavage in Dex-resistant OPM1 cells.

### Example 6

### Combined R-Etodolac with Dex has Synergistic In Vivo Anti-MM Activity

MM Xenograft Murine Model

CB-17 SCID-mice (Taconic, Gemantown, N.Y.) were subcutaneously inoculated in the interscapular area with 2×10⁶ OPM1 cells in 100 μl of RPMI-1640 medium. When tumor was measurable, approximately 3 weeks after MM cell injection, mice were treated for 13 consecutive days with either vehicle alone or R-etodolac (250 mg/kg) orally, with and without i.p. Dex (1 mg/kg). Tumor size was measured every other day in 2 dimensions using a caliper. Tumor volume was calculated using the formula: V=0.5 a×b², where a and b are the long and short diameter of the tumor, respectively. Animals were sacrificed when their tumors reached 2 cm.

Results:

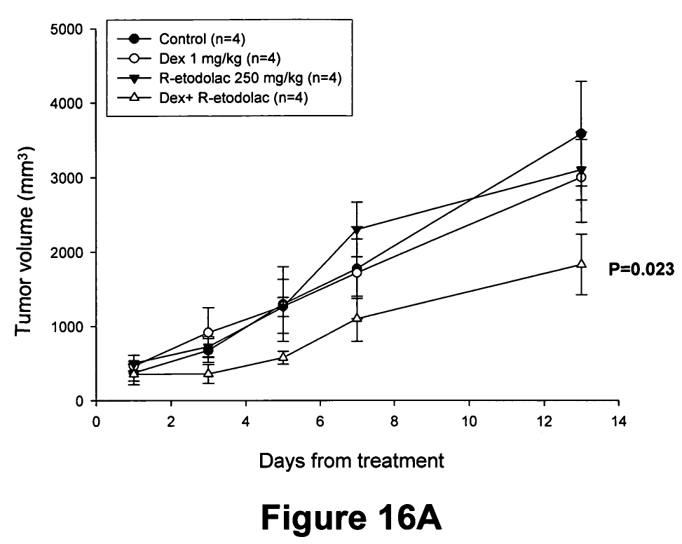

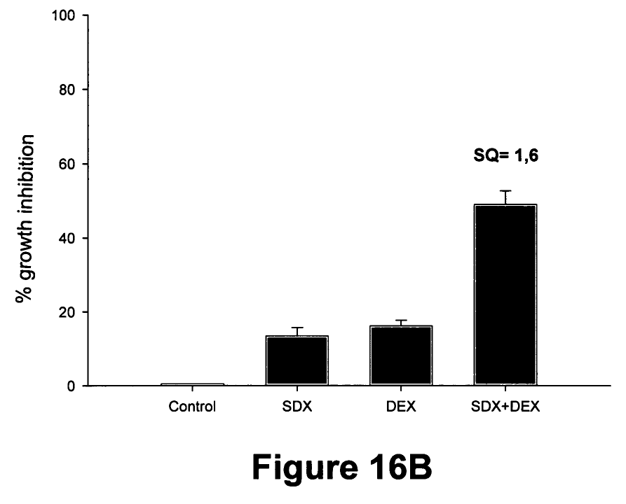

The in vivo anti-tumor effect of combined R-etodolac and Dex using SCID mice injected subcutaneously with Dex-resistant OPM1 MM cells was investigated. A cohort of 16 mice engrafted with OPM1 were treated daily with Dex i.p. and/or R-etodolac orally for 13 days, and tumor burden was assessed on alternate days using an electronic caliper. As shown in FIG. 16A, treatment with R-etodolac alone (250 mg/kg/day) or Dex alone (1 mg/kg/day) did not induce significant reduction of tumor volume compared with control, whereas the combination of R-etodolac and Dex induced significant tumor regression (p=0.023). Moreover, as shown in FIG. 16B, this combination resulted in a synergistic anti-proliferative effect (SQ=1.60). These results demonstrate that treatment with R-etodolac or Dex alone does not induce reduction of tumor volume compared with control, whereas the combination of R-etodolac and Dex significantly and synergistically inhibits tumor growth in vivo.

All patents and documents referenced are incorporated herein by reference to the extent they are not inconsistent with the present disclosure.

The invention is not limited to those embodiments described herein, but may encompass modification and variations which do not depart from the spirit of the invention. While the invention has been described in connection with specific embodiments thereof, those of ordinary skill in the art will understand that further modifications are within the scope of the following claims. In addition, where features or aspects of the invention are described in terms of Markush groups or other grouping of alternatives, those skilled in the art will recognize that the invention is also thereby described in terms of any individual member or subgroup of members of the Markush group or genus, and exclusions of individual members as appropriate.

What is claimed is:

1. A method for treating refractory or resistant multiple myeloma comprising administering to a subject in need thereof a therapeutically effective amount of an NSAID or NSAID analog or an enantiomer of said NSAID or NSAID analog.

2. A method for treating refractory or resistant multiple myeloma comprising administering to a subject in need thereof a therapeutically effective amount of a β-catenin inhibitor or a therapeutically effective amount of a cyclin D inhibitor.

3. A method for treating refractory or resistant multiple myeloma comprising administering to a subject in need thereof a therapeutically effective amount of a compound of formula (I) or a enantiomer of said compound 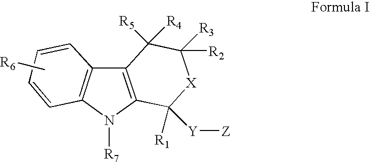
in which R₁ is selected from the group consisting of hydrogen, lower alkyl, lower alkenyl, lower alkynyl, lower cycloalkyl, phenyl, benzyl and 2-thienyl; R₂, R₃ R₄ and R₅ are the same or different and are each selected from the group consisting of hydrogen and lower alkyl, NH₂, —NHCHO, —NHCONH₂, ═NW, OXO, —OH and —OCH₃, wherein W is hydroxy, alkoxy, aryloxy, carboxyalkyloxy, arylamino or alkylsulfonylamino; R₆ is selected from the group consisting of hydrogen, lower alkyl, lower alkenyl, lower alkynyl, trifluoromethyl, hydroxy, lower alkoxy, trifluoroloweralkoxy, aryloxy, benzyloxy, aralkoxy, lower alkanoyloxy, acyl, amino, nitro, cyano, alkylimido, halo, mercapto, loweralkylthio, alkylsulfinyl, alkylsulfonyl, alkylsulfonamido and sulfamoyl; R₇ is selected from the group consisting of hydrogen, lower alkyl and lower alkenyl; X is selected from the group consisting of carbon, oxy and thio; Y is selected from the group consisting of carbonyl, 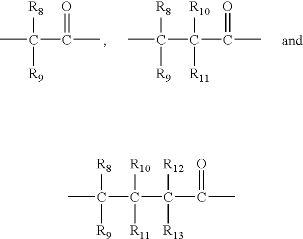
in which each of R₈, R₉, R₁₀, R₁₁, R₁₂ and R₁₃ is hydrogen or lower alkyl; and Z is selected from the group consisting of hydroxy, lower alkoxy, amino, lower alkylamino, di(lower)alkylamino and phenylamino,
or a pharmaceutically acceptable salt thereof.

4. A method according to claim 3 wherein the compound is etodolac.

5. A method according to claim 3 wherein the compound is R-etodolac.

6. A method for treating refractory or resistant multiple myeloma comprising administering to a subject in need thereof a therapeutically effective amount of a compound of Formula (II) or enantiomer of said compound 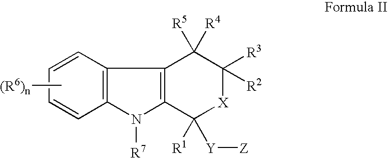
wherein R¹ is lower alkyl, lower alkenyl, (hydroxy)lower alkyl, lower alkynyl, phenyl, benzyl or 2-thienyl; R², R³, R⁴ and R⁵ are the same or different and are each hydrogen or lower alkyl; each R⁶ is independently hydrogen, lower alkyl, hydroxy, (hydroxy)lower alkyl, lower alkoxy, benzyloxy, lower alkanoyloxy, nitro or halo and n is 1-3; R⁷ is hydrogen, lower alkyl or lower alkenyl; X is carbon, oxy or thio; Y is carbonyl, (CH₂)₁₋₃C(O)—, —(CH₂)₁₋₃—, or —CH₂)₁₋₃SO₂—; and Z is hydroxy, lower alkoxy, (C₂-C₄)acyloxy, —N(R⁸)(R⁹), phenylamino, (ω-(4-pyridyl)(C₂-C₄ alkoxy), (ω-((R⁸)(R⁹) amino)(C₂-C₄ alkoxy), an amino acid ester of (ω-(HO)(C₂-C₄))alkoxy, —N(R⁸)CH(R⁸)CO₂H, 1′-D-glucuronyloxy, —SO₃H, —PO₄H₂, —N(NO)(OH), —SO₂NH₂, —PO(OH)(NH₂), —OCH₂CH₂N(CH₃)₃⁺, or tetrazolyl; wherein R⁸ and R⁹ are each hydrogen, or (C₁-C₃)alkyl; or R⁸ and R⁹ together with N, form a 5- or 6-membered heterocyclic ring having 1-3 N(R⁸), S or non-peroxide O; or a pharmaceutically acceptable salt thereof.

7. A method for treating refractory or resistant multiple myeloma comprising administering to a subject in need thereof a therapeutically effective amount of a compound of Formula (III) 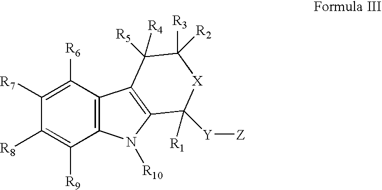
wherein:
(a) X is C, S or O;
(b) R₁ is hydrogen; halogen; —CN; —OH; —SH; —NO₂; or an unsubstituted or substituted moiety selected from alkyl, alkenyl, alkynyl, aryl, heteroaryl, heterocycloalkyl, and cycloalkyl;
(c) R₂, R₃, R₄ and R₅ are each independently hydrogen; halogen; —CN; —OH; —SH; —NO₂; or an unsubstituted or substituted moiety selected from lower alkyl, lower alkynyl, lower alkenyl, alkoxy, haloalkyl, aryl, and heteroaryl;
(d) R₆, R₇, R₈ and R₉ are each independently hydrogen; halogen; —CN; —OH; —SH; —NO₂; or an unsubstituted or substituted moiety selected from alkyl, alkenyl, alkynyl, alkoxy, allyloxy, aryl, heteroaryl, cycloalkyl, and heterocycloalkyl, wherein at least one of R₆, R₇, R₈ and R₉ is an unsubstituted or substituted moiety selected from aryl, heteroaryl, cycloalkyl, heterocycloalkyl, alkenyl, and alkynyl;
(e) R₁₀ is hydrogen; or an unsubstituted or substituted moiety selected from lower alkyl, lower alkenyl, lower alkynyl, aryl; heteroaryl, heterocycloalkyl, and cycloalkyl;
(f) Y is an unsubstituted or substituted moiety selected from alkyl, alkenyl, and alkynyl; and
(g) Z is a moiety selected from —OH, —NH₂, —SH, —SO₂OH, —S(O)H, —OC(O)NH₂, —S(O)₂NH₂, —NHC(O)H, C(O)NH₂, unsubstituted or substituted with one, two or three suitable substituents each independently selected from the group consisting of alkyl, haloalkyl, heteroalkyl, aryl, heteroaryl, cycloalkyl, and heterocycloalkyl;
wherein R₁ and Y may cyclize to form an unsubstituted or substituted cycloalkyl group or an unsubstituted or substituted heterocycloalkyl group;
or a pharmaceutically acceptable prodrug, pharmaceutically active metabolite, or pharmaceutically acceptable salt thereof.

8. A method according to claim 7 wherein:
(a) X is S or O;
(b) R₁ is hydrogen; or an unsubstituted moiety selected from lower alkyl, lower alkyl-hydroxy, lower alkenyl, lower alkenyl-hydroxy, lower alkynyl, lower alkynyl-hydroxy, aryl, heteroaryl, heterocycloalkyl, and cycloalkyl;
(c) R₂, R₃, R₄ and R₅ are each independently hydrogen; or an unsubstituted moiety selected from lower alkyl, lower alkynyl, lower alkenyl, alkoxy, haloalkyl, aryl, and heteroaryl;
(d) R₆, R₈ and R₉ are each independently hydrogen; or an unsubstituted or substituted moiety selected from alkyl, alkenyl, alkynyl, alkoxy, allyloxy, aryl, heteroaryl, cycloalkyl, and heterocycloalkyl, wherein the substituted moieties are each independently selected from the group consisting of halogen, —CN, alkyl, alkoxy, —NH₂, —O-haloalkyl, —CH(O), haloalkyl, aryl, heteroaryl, heterocycloalkyl, alkenyl, alkynyl, —OH, —C(O)₂-alkyl, and —C(O)₂H;
(e) R₇ is hydrogen; halogen; —CN; —OH; —SH; —NO₂; unsubstituted lower alkyl, unsubstituted lower alkenyl, unsubstituted lower alkynyl, alkyl-C(O)₂H, alkyl-C(O)₂-alkyl, or lower alkoxy; and
(f) R₁₀ is hydrogen; or an unsubstituted moiety selected from lower alkyl, lower alkenyl, lower alkynyl, aryl, benzyl, heteroaryl, heterocycloalkyl, and cycloalkyl.

9. A method according to claim 7 wherein:
(a) R₉ is hydrogen, halogen or an unsubstituted alkyl group;
(b) Y is an unsubstituted alkyl group; and
(c) Z is hydroxyl.

10. A method according to claim 7 wherein:
(a) X is O or S;
(b) R₁ is an unsubstituted lower alkyl group;
(c) R₂, R₃, R₄ and R₅ are each hydrogen;
(d) R₆ is hydrogen or halogen;
(e) R₇ is halogen, unsubstituted lower alkyl, lower alkyl-C(O)₂H, lower alkyl-C(O)₂-lower alkyl, or lower alkoxy;
(f) R₈ is hydrogen or halogen;
(g) R₉ is hydrogen; or an unsubstituted or substituted moiety selected from alkenyl, alkynyl, aryl, heteroaryl, cycloalkyl, and heterocycloalkyl; and
(h) R₁₀ is hydrogen.

11. A method according to claim 10 wherein Y is an unsubstituted lower alkyl group and Z is hydroxyl.

12. A method according to claim 7 wherein:
(a) X is O;
(b) R₁ is an unsubstituted moiety selected from aryl, alkyl, and lower-alkoxy;
(c) R₂, R₃, R₄, and R₅ are each hydrogen;
(d) R₆ and R₈ are each hydrogen or halogen;
(e) R₇ is halogen, unsubstituted lower alkyl, lower alkyl-C(O)₂H, lower alkyl-C(O)₂-lower alkyl, or lower alkoxy;
(f) R₉ is an unsubstituted branched alkyl group; and
(g) R₁₀ is hydrogen.

13. A method according to claim 12 wherein:
(a) Y is an unsubstituted lower alkyl group; and
(b) Z is hydroxyl.

14. A method for treating refractory or resistant multiple myeloma comprising administering to a subject in need thereof a therapeutically effective amount of a compound of Formula (III) 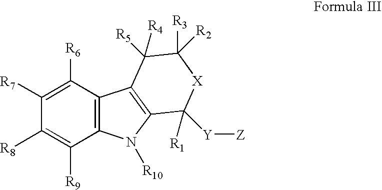
wherein:
(a) X is C, S or O;
(b) R₁ is hydrogen; halogen; —CN; —OH; —SH; —NO₂; or an unsubstituted or substituted moiety selected from alkyl, alkenyl, alkynyl, aryl, heteroaryl, heterocycloalkyl, and cycloalkyl, wherein the substituted groups are substituted with one, two or three suitable substituents each independently selected from the group consisting of: halogens, —CN, —NO₂, unsubstituted alkyl, unsubstituted alkenyl, unsubstituted heteroalkyl, unsubstituted haloalkyl, unsubstituted alkynyl, unsubstituted aryl, unsubstituted cycloalkyl, unsubstituted heterocycloalkyl, unsubstituted heteroaryl, and —(CH₂)_{z}CN where z is an integer from 0 to 6;
(c) R₂, R₃, R₄ and R₅ are each independently hydrogen; halogen; —CN; —OH; —SH; —NO₂; or an unsubstituted or substituted moiety selected from lower alkyl, lower alkynyl, lower alkenyl, alkoxy, haloalkyl, aryl, and heteroaryl;
(d) R₆, R₈ and R₉ are each independently hydrogen; halogen; —CN; —OH; —SH; —NO₂; or an unsubstituted or substituted moiety selected from alkyl, alkenyl, alkynyl, alkoxy, allyloxy, aryl, heteroaryl, cycloalkyl, and heterocycloalkyl;
(e) R₇ is hydrogen; halogen; —CN; —OH; —SH; —NO₂; or an unsubstituted or substituted moiety selected from alkyl, alkenyl, and alkynyl.
(f) R₁₀ is hydrogen; or an unsubstituted or substituted moiety selected from lower alkyl, lower alkenyl, lower alkynyl, aryl; heteroaryl, heterocycloalkyl, and cycloalkyl;
(g) Y is an unsubstituted or substituted moiety selected from alkyl, alkenyl, and alkynyl; wherein the substituted moiety is substituted with one, two or three substituents each independently selected from halogen; —CN; —OH; —SH; —NO₂; unsubstituted alkyls, unsubstituted alkenyls, unsubstituted alkynyls, unsubstituted heteroalkyls, unsubstituted haloalkyls, unsubstituted aryls, unsubstituted cycloalkyls, unsubstituted heterocycloalkyls, and unsubstituted heteroaryls; and
(h) Z is a moiety selected from —OH, —SH, —OC(O)NH₂;
wherein R₁ and Y may cyclize to form an unsubstituted or substituted cycloalkyl group or an unsubstituted or substituted heterocycloalkyl group; and at least one of R₆, R₇, R₈ and R₉ is not hydrogen;
or a pharmaceutically acceptable salt thereof.

15. A method for treating refractory or resistant multiple myeloma comprising administering to a subject in need thereof a therapeutically effective amount of a compound of Formula (III) 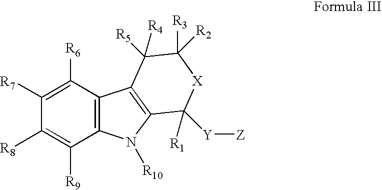
wherein:
(a) X is C, S or O;
(b) R₁ is hydrogen; halogen; —OH; —SH; —CN; —NO₂; or an unsubstituted or substituted moiety selected from alkyl, alkenyl, alkynyl, aryl, heteroaryl, heterocycloalkyl, and cycloalkyl, wherein the substituted groups are substituted with one, two or three suitable substituents each independently selected from the group consisting of: halogens, —CN, —NO₂, —SH, —OH, unsubstituted alkyl, unsubstituted alkenyl, unsubstituted heteroalkyl, unsubstituted haloalkyl, unsubstituted alkynyl, unsubstituted aryl, unsubstituted cycloalkyl, unsubstituted heterocycloalkyl, unsubstituted heteroaryl, and —(CH₂)_{z}CN where z is an integer from 0 to 6;
(c) R₂, R₃, R₄ and R₅ are each independently hydrogen; halogen; —OH; —SH; —CN; —NO₂; or an unsubstituted or substituted moiety selected from lower alkyl, lower alkynyl, lower alkenyl, alkoxy, haloalkyl, aryl, and heteroaryl;
(d) R₆, R₇, R₈ and R₉ are each independently hydrogen; halogen; —OH; —SH; —CN; —NO₂; or an unsubstituted or substituted moiety selected from alkyl, alkenyl, alkynyl, alkoxy, allyloxy, aryl, heteroaryl, cycloalkyl, and heterocycloalkyl, wherein at least one of R₆, R₇, R₈ and R₉ is an unsubstituted or substituted moiety selected from aryl, heteroaryl, cycloalkyl, heterocycloalkyl, alkenyl, and alkynyl;
(e) R₁₀ is hydrogen; or an unsubstituted or substituted moiety selected from lower alkyl, lower alkenyl, lower alkynyl, aryl; heteroaryl, heterocycloalkyl, and cycloalkyl;
(f) Y is an unsubstituted or substituted moiety selected from alkyl, alkenyl, and alkynyl; wherein the substituted moiety is substituted with one, two or three substituents each independently selected from halogen; —OH; —SH; —CN; —NO₂; unsubstituted alkyls, unsubstituted haloalkyls, unsubstituted heteroalkyls, unsubstituted alkenyls, unsubstituted alkynyls, unsubstituted aryls, unsubstituted cycloalkyls, unsubstituted heterocycloalkyls, and unsubstituted heteroaryls; and
(g) Z is a moiety selected from —OH, —SH, —OC(O)NH₂, —SO₂H, —SO₂NH₂, —SO₂OH, —S(O)H, —NH₂, —NHC(O)H, C(O)NH₂, unsubstituted or substituted with one or two suitable substituents selected from the group consisting of alkyl, haloalkyl, heteroalkyl, aryl, heteroaryl, cycloalkyl, and heterocycloalkyl each independently substituted with one, two or three suitable substituents;
wherein R₁ and Y may cyclize to form an unsubstituted or substituted cycloalkyl group or an unsubstituted or substituted heterocycloalkyl group;
or pharmaceutically acceptable salt thereof.

16. A method for treating refractory or resistant multiple myeloma comprising administering to a subject in need thereof a therapeutically effective amount of a compound selected from the group consisting of: 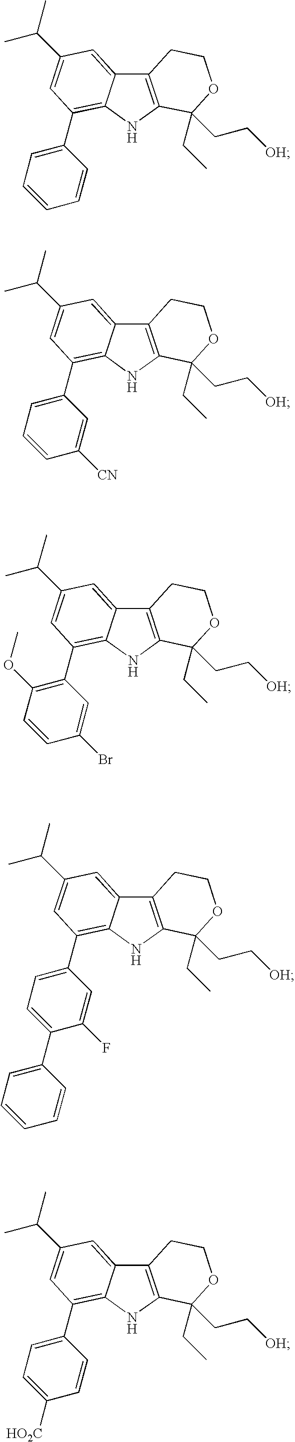 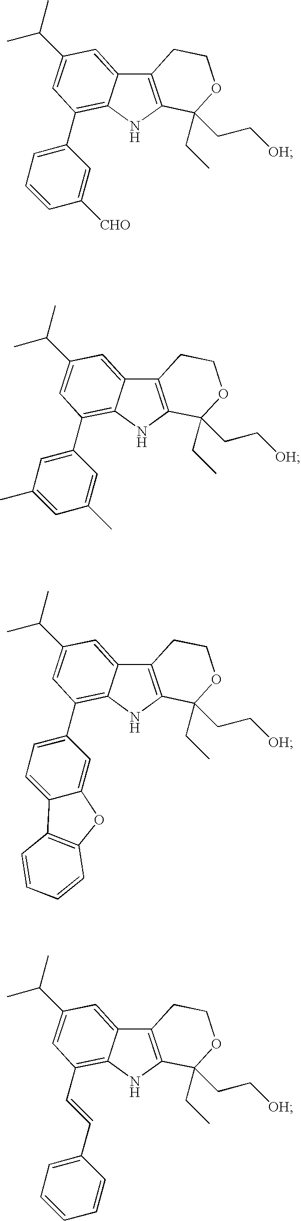 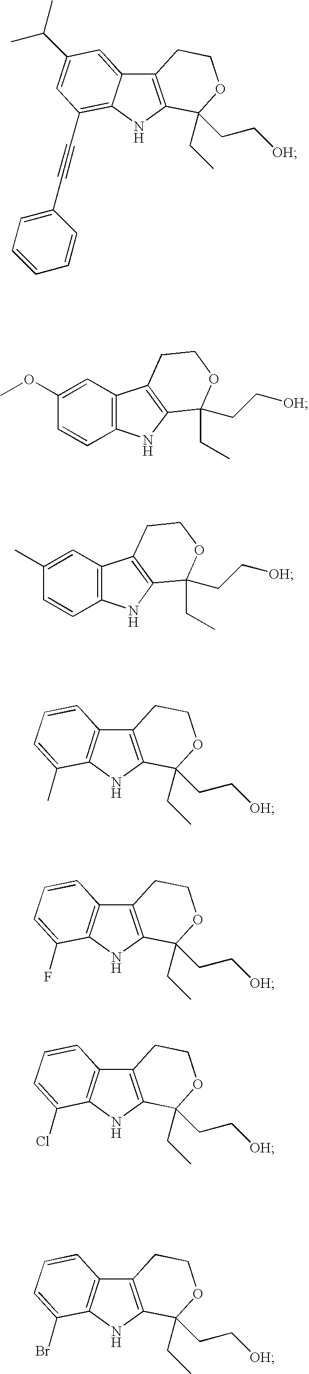 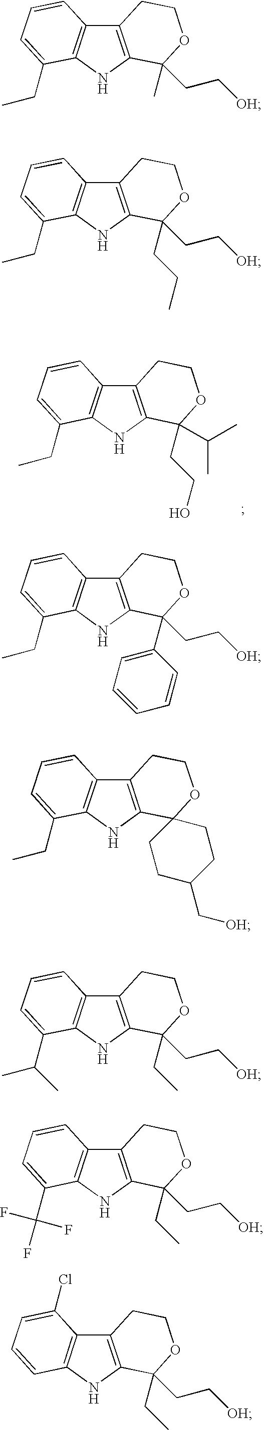 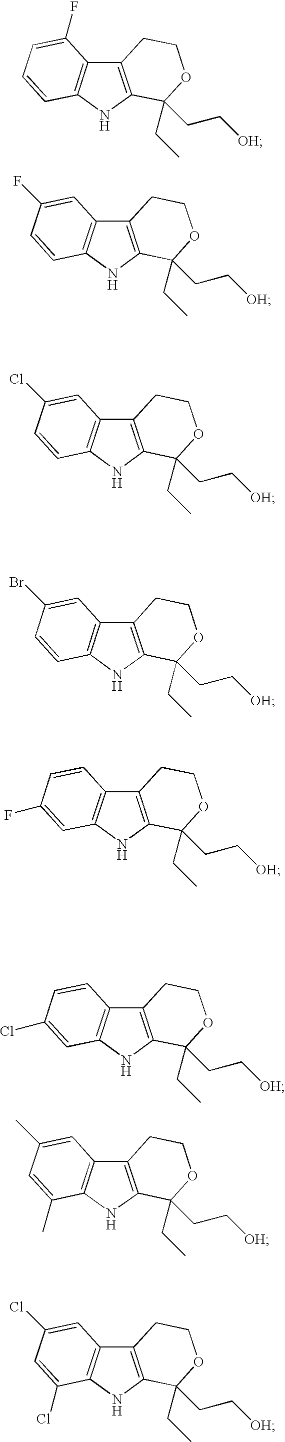 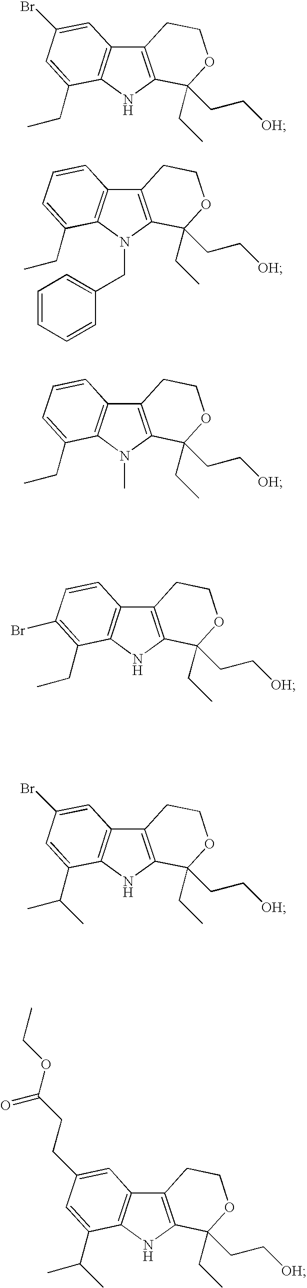 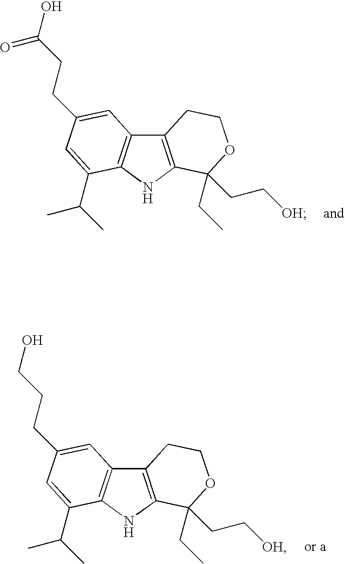
pharmaceutically acceptable salt thereof.

17. A method for treating refractory or resistant multiple myeloma comprising administering to a subject in need thereof a compound selected from the group consisting of: 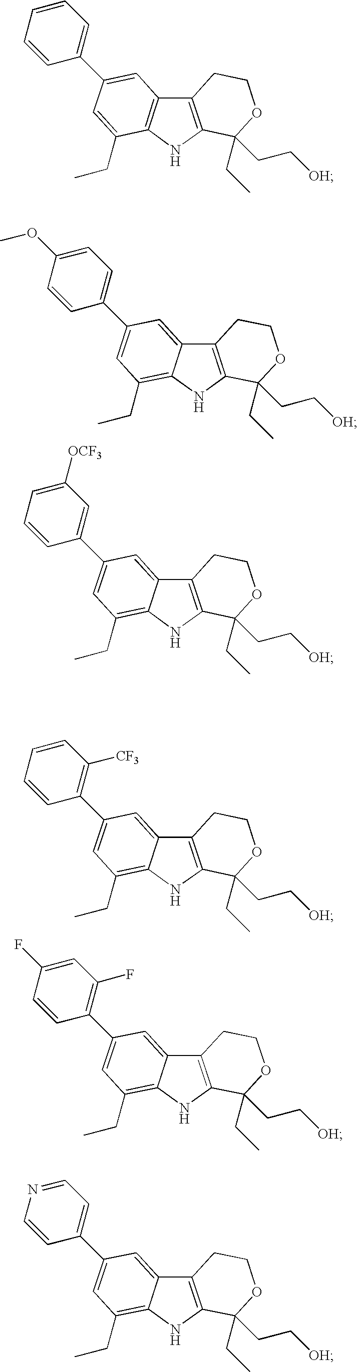 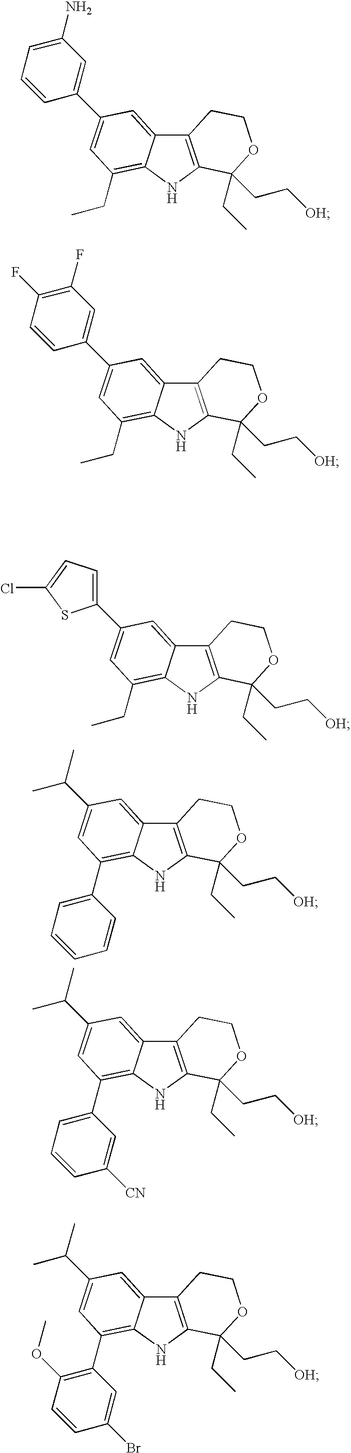 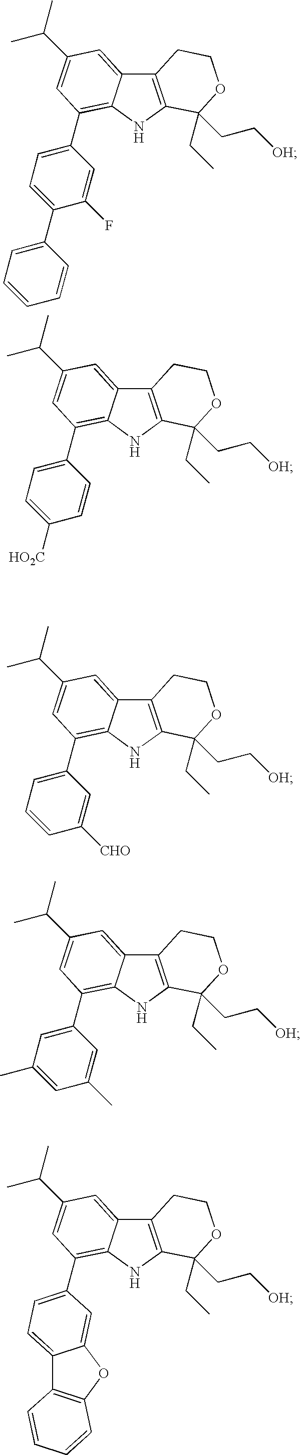 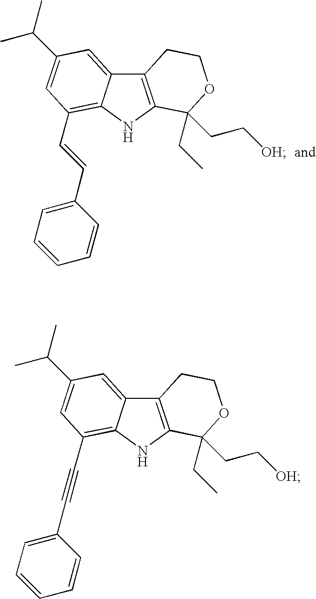
or a pharmaceutically acceptable salt thereof.

18. A method for treating refractory or resistant multiple myeloma comprising administering to a subject in need thereof a compound having the following structure: 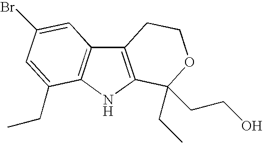
or a pharmaceutically acceptable salt thereof.

19. A method for treating refractory or resistant multiple myeloma comprising administering to a subject in need thereof a compound having the following structure: 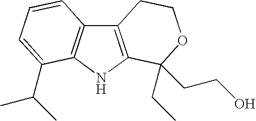
or a pharmaceutically acceptable salt thereof.

20. A method for treating refractory or resistant multiple myeloma comprising administering to a subject in need thereof a compound having the following structure: 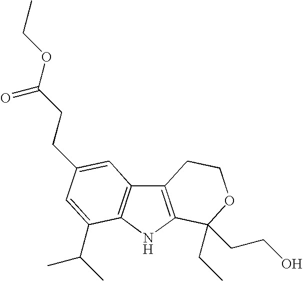
or a pharmaceutically acceptable salt thereof.

21. A method for treating refractory or resistant multiple myeloma comprising administering to a subject in need thereof a compound having the following structure: 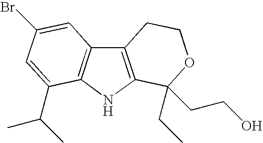
or a pharmaceutically acceptable salt thereof.

22. A method for treating refractory or resistant multiple myeloma comprising administering to a subject in need thereof a compound having following structure: 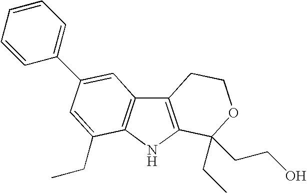
or a pharmaceutically acceptable salt thereof.

23. A method for treating refractory or resistant multiple myeloma comprising administering to a subject in need thereof a compound of claim 1, claim 2, claim 3, claim 4, claim 6, or claim 7 in combination with one or more antineoplastic agents.

24. A method according to claim 23, wherein the antineoplastic agent is selected from one or more of the group consisting of vincristine, doxorubicin, dexamethasone, thalidomide, thalidomide derivatives, 2ME2, Neovastat, R 11 5777, arsenic trioxide, bortezomib, tamoxifen, G3139 (antisense), and SU5416.

25. A method according to claim 24, wherein the antineoplastic agent is dexamethasone.

26. A method according to claim 24, wherein the antineoplastic agent is arsenic trioxide.

27. A method according to claim 24, wherein the antineoplastic agent is bortezomib.

28. A method according to claim 24, wherein the antineoplastic agent is thalidomide.

29. A method according to claim 24, wherein the antineoplastic agent is a thalidomide derivative.

30. A method for treating refractory or resistant multiple myeloma comprising administering to a subject that has failed VAD therapy a compound of claim 1, claim 2, claim 3, claim 4, claim 6, or claim 7.

31. A method according to claim 1, claim 2, claim 3, claim 4, claim 6, or claim 7 wherein the subject is human.

32. A method according to claim 1, wherein the refractory or resistant myeloma is resistant to glucocorticoids.

33. A method according to claim 32, wherein the glucocorticoids is dexamethasone.

34. A method according to claim 1, wherein the refractory or resistant multiple myeloma is resistant to one or more of the group consisting of dexamethasone, doxorubicin, melphalan, and bortexomib.

35. A method according to claim 34, wherein the refractory or resistant multiple myeloma is resistant to doxorubicin.

36. A method according to claim 34, wherein the refractory or resistant multiple myeloma is resistant to bortezomib.

37. A method according to claim 34, wherein the refractory or resistant multiple myeloma is resistant to melphalan.

38. A method of treating resistant or refractory multiple myeloma comprising administering to a patient an antineoplastic agent that induces upregulation of Mcl-1s.

39. A method according to claim 38, wherein the antineoplastic agent is a compound selected from claim 3, 6, or 7.

40. A method according to claim 39, wherein the antineoplastic agent is R-etodolac.

41. A method for selecting a compound for use in treating multiple myeloma comprising contacting multiple myeloma cells with a test compound and determining if said compound induces the upregulation of Mcl-1s, wherein if said compound induces upregulaton of Mcl-1s it is a candidate as a drug for use in treating multiple myeloma.

42. A method according to claim 41, wherein the multiple myeloma cells used for selection are resistant to one or more agents used to treat multiple myeloma.

43. A method for promoting the sale of a compound from claim 3, 6, or 8 comprising distributing and or discussing the use of such compounds for treating resistant or refractory multiple myeloma to a person or entity that supplies such compounds to hospitals and or medical personal for use in treating subjects afflicted with resistant or refractory multiple myeloma.

44. Use of an NSAID or NSAID analog or an enantiomer of said NSAID or NSAID analog in the manufacture of a medicament for treatment of refractory or resistant multiple myeloma.
